(19) United States
(12) Patent Application Publication
(10) Pub. No.: US 2016/0166671 A1
(43) Pub. Date: Jun. 16, 2016

(54) Clostridium Difficile Compositions and Methods of Use

(71) Applicant: VaxInnate Corporation, Cranbury, NJ (US)

(72) Inventors: Lynda Tussey, Cranbury, NJ (US); Langzhou Song, Cranbury, NJ (US); Scott Umlauf, Cranbury, NJ (US)

(21) Appl. No.: 14/875,789

(22) Filed: Oct. 6, 2015

Related U.S. Application Data
(63) Continuation of application No. PCT/US2014/033334, filed on Apr. 8, 2014.
(60) Provisional application No. 61/853,635, filed on Apr. 9, 2013.

(51) Int. Cl. A61K 39/08; C07K 16/12; A61K 38/16

(52) U.S. Cl. CPC A61K 39/08; A61K 38/164; C07K 16/1282; A61K 2039/55

(57) ABSTRACT

Fusion proteins include full-length flagellin or portions of flagellin fused to at least one Clostridium difficile antigen. Fusion proteins can include a first Clostridium difficile antigen that replaces at least a portion or the entirety of domain 3 of flagellin and a second Clostridium difficile antigen fused to the carboxy terminal amino acid of flagellin. Fusion proteins can also include at least one Clostridium difficile antigen fused to the most carboxy terminus of portions of flagellin or full length flagellin. Methods that employ fusion proteins are administered to humans to ameliorate Clostridium difficile associated disease.

### RELATED APPLICATIONS

This application is a continuation of International Application No. PCT/2014/033334, which designated the United States and was filed on Apr. 8, 2014, published in English, which claims the benefit of U.S. Provisional Application No. 61/853,635, filed on Apr. 9, 2013. The entire teachings of the above applications are incorporated herein by reference.

### INCORPORATION BY REFERENCE OF MATERIAL IN ASCII TEXT FILE

This application incorporates by reference the Sequence Listing contained in the following ASCII text file being submitted concurrently herewith:
a) File name: 37101058003SEQUENCE LISTING.txt; created Sep. 18, 2015, 666 KB in size.

### BACKGROUND OF THE INVENTION

Clostridium difficile is a spore-forming, gastrointestinal pathogen that is a leading cause of nosocomial infections in developed nations. Ingested spores survive passage through the stomach and ultimately reside in the colon. Clostridium difficile associated diarrhea typically occurs when antimicrobial therapy, such as the use of antibiotics, creates an ecological niche that allows the spores to germinate. As the Clostridium difficile bacteria colonize the colon, they produce multiple exotoxins toxin A (TcdA), toxin B (TcdB) and binary toxins. The Clostridium difficile pathogen can affect elderly patients, in particular during hospitalizations that lead to severe Clostridium difficile associated diarrhea and pseudomembranous colitis with case-fatality rates of up to about 10%.

Each year more than 300,000 cases of Clostridium difficile-associated diarrhea are reported in the U.S. alone and recently hypervirulent and antibiotic-resistant Clostridium difficile strains have emerged that are associated with increased morbidity, mortality and recurrence rates. For mild to moderate Clostridium difficile associated diarrhea, treatment with metronidazole or oral vancomycin is the standard of care, but recurrences of Clostridium difficile associated diarrhea are common with these treatments. While the elderly are still most affected, Clostridium difficile associated disease has been reported in what have been considered traditionally ‘low risk’ humans, such as healthy persons in the community, peripartum women and children. Currently there are no commercially available compositions for ameliorating or preventing Clostridium difficile associated disease and infection. A common treatment for Clostridium difficile associated diseases is the use of antibiotics. However, antibiotic resistant strains of Clostridium difficile are emerging. Therefore, there is a need to develop new and useful compositions and therapeutic methods to ameliorate or prevent Clostridium difficile associated disease.

### SUMMARY OF THE INVENTION

The present invention relates to fusion proteins that include flagellin and at least one Clostridium difficile antigen and methods of treating humans with compositions that include fusion proteins comprising flagellin and at least one Clostridium difficile antigen.

In an embodiment, the invention is a composition comprising a first fusion protein that activates Toll-like Receptor 5 and includes flagellin fused to at least one Clostridium difficile toxin A antigen; and a second fusion protein that activates Toll-like Receptor 5 and includes flagellin fused to at least one Clostridium difficile toxin B antigen.

In yet another embodiment, the invention is a composition comprising a first fusion protein that activates Toll-like Receptor 5 and includes flagellin fused to at least one Clostridium difficile toxin A antigen; a second fusion protein that activates Toll-like Receptor 5 and includes flagellin fused to at least one Clostridium difficile toxin B antigen; and at least one additional fusion protein that comprises flagellin fused to at least one Clostridium difficile binary toxin antigen.

In an embodiment, the invention is a fusion protein comprising a flagellin protein in which domain 3 of the flagellin protein has been replaced with a first Clostridium difficile antigen and a second Clostridium difficile antigen is fused to the carboxy-terminal amino acid of the flagellin protein, wherein the fusion protein activates Toll-like Receptor 5.

In an embodiment, the invention is a fusion protein comprising a flagellin protein in which at least a portion of domain 3 of the flagellin protein has been replaced with a first Clostridium difficile antigen and a second Clostridium difficile antigen is fused to the carboxy-terminal amino acid of the flagellin protein, wherein the fusion protein activates Toll-like Receptor 5.

In another embodiment, the invention is a fusion protein comprising a full length flagellin or a portion of a flagellin fused at the carboxy-terminal amino acid to at least one Clostridium difficile antigen selected from the group consisting of a portion or the entirety of a receptor binding domain of Clostridium difficile, a portion or the entirety of a translocation domain of Clostridium difficile and a portion of the entirety of a glucosyltransferase domain.

In still another embodiment, the invention is a fusion protein comprising a full length flagellin or a portion of a flagellin fused at the amino-terminal amino acid to at least one Clostridium difficile antigen selected from the group consisting of a portion or the entirety of a receptor binding domain of Clostridium difficile, a portion or the entirety of a translocation domain of Clostridium difficile and a portion of the entirety of a glucosyltransferase domain.

In a further embodiment, the invention is method of treating a human, comprising the step of administering to the human at least one fusion protein that includes a full length flagellin or a portion of a flagellin fused at the carboxy-terminal amino acid to at least one Clostridium difficile antigen selected from the group consisting of a portion or the entirety of a receptor binding domain of Clostridium difficile, a portion or the entirety of a translocation domain of Clostridium difficile and a portion of the entirety of a glucosyltransferase domain, whereby administration of the composition ameliorates a Clostridium difficile associated disease.

In yet another embodiment, the invention is a method of treating a human, comprising the step of administering to the human at least one fusion protein that activates Toll-like Receptor 5 and includes a flagellin protein in which domain 3 of the flagellin protein has been replaced with a first Clostridium difficile antigen and a second Clostridium difficile antigen is fused to the carboxy-terminal amino acid of the flagellin protein, whereby administration of the composition ameliorates a Clostridium difficile associated-disease.

In yet another embodiment, the invention is method of treating a human, comprising the step of administering to the human at least one fusion protein that includes a full length flagellin or a portion of a flagellin fused at the amino-terminal amino acid to at least one Clostridium difficile antigen selected from the group consisting of a portion or the entirety of a receptor binding domain of Clostridium difficile, a portion or the entirety of a translocation domain of Clostridium difficile and a portion of the entirety of a glucosyltransferase domain, whereby administration of the composition ameliorates a Clostridium difficile associated disease.

In yet another embodiment, the invention is a method of treating a human, comprising the step of administering to the human at least one fusion protein that activates Toll-like Receptor 5 and includes a flagellin protein in which domain 3 of the flagellin protein has been replaced with a first Clostridium difficile antigen and a second Clostridium difficile antigen is fused to the amino-terminal amino acid of the flagellin protein, whereby administration of the composition ameliorates a Clostridium difficile associated-disease.

The fusion proteins and methods of treating humans with compositions that include the fusion proteins of the invention ameliorate a Clostridium difficile associated disease, such as by generating neutralizing antibodies in the human to at least one Clostridium difficile antigen. Ameliorating Clostridium difficile associated disease can be advantageous in hospitalizations where patients are particularly susceptible to disease consequent to a reduction in intestinal flora after antibiotic treatment. The fusion proteins and methods of treating humans with fusion proteins of the invention may avoid serious illness and death consequent to Clostridium difficile associated disease.

### BRIEF DESCRIPTION OF THE DRAWINGS

FIGS. 1A, 1B and 1C depict domains of toxin A (TcdA) (FIG. 1A), toxin B (TcdB) (FIG. 1B) and binary toxin (FIG. 1C) produced by Clostridium difficile. Toxins A and B (TcdA and TcdB) are single-chain proteins with discreet domains. GTD=glucosyltransferase domain, also referred to as the “catalytic domain.” CPD=cysteine protease domain. TLD=translocation domain. RBD=receptor binding domain. Numbers depict amino acid residues of SEQ ID NO: 15 for toxin A (FIG. 1A) and of SEQ ID NO: 16 for toxin B (FIG. 1B).

FIG. 2 depicts the domains (DO, Dl, D2, D3) of an exemplary full length flagellin (FL, STF2, SEQ ID NO: 54) and a fusion protein that includes Domains 0, 1, 2 and 3 and a Clostridium difficile antigen (Ag) fused to the carboxy-terminus of the flagellin (also referred to as “C-terminal” or “C-term” format).

FIG. 3 depicts the domains (DO, Dl, D2, D3) of an exemplary flagellin construct (SEQ ID NO: 55) and a fusion protein that includes, in sequence, the amino-domain 0, the amino-domain 1, the amino-domain 2, the carboxy-domain 2, the carboxy-domain 1 and the carboxy-domain 0 of flagellin. A Clostridium difficile antigen is fused between the amino domain 2 and carboxy domain 2 of the flagellin construct, which results in replacement of domain 3 of flagellin (after amino acid residue Ala 190) with a Clostridium difficile antigen. The flagellin construct is referred to herein as “the R3 construct” or “R3 formats.”

FIG. 4 depicts the domains (DO, Dl, D2, D3) of an exemplary flagellin construct (SEQ ID NO: 55) and a fusion protein that includes, in sequence, the amino-domain 0 (D0N), the amino-domain 1 (DIN) , the amino-domain 2 (D2N), the carboxy-domain 2 (D2C), the carboxy-domain 1 (D1C) and the carboxy-domain 0 (D0C) of the flagellin. A first Clostridium difficile antigen replaces domain 3 (D3) of flagellin after amino acid residue 190) and a second Clostridium difficile antigen is fused to the carboxy-terminus amino acid of flagellin. This flagellin construct is referred to herein as “the R3-2xAg construct,” “R32x format” or R3.2x format.”

FIG. 5 depicts exemplary fusion proteins of the invention.

FIG. 6 depicts capture enzyme-linked immunosorbent assay (ELISA) results for C. difficile Toxin A/flagellin fusion proteins. Comparison of R3.2x and C-terminal formats. ELISA plates were coated with polyclonal chicken egg anti-Toxin A IgY (Gallus Immunotech). Serially diluted fusion proteins (R3.2x format HL821, SEQ ID NO: 9 and C-terminal format HL928, SEQ ID NO: 25) were then bound and detected with polyclonal goat anti-flagellin IgG (Covance Research Products). Plates were developed with rabbit anti-goat IgG conjugated to HRP (Jackson Immunoresearch), TMB and H₂SO₄. Curves were fit with a 4-parameter logistic equation (Softmax Pro 5.4).

FIG. 7 depicts capture ELISA for C. difficile Toxin B/flagellin fusion proteins. Comparison of R3.2x and C-terminal formats. ELISA plates were coated with polyclonal chicken egg anti-Toxin B IgY (Gallus Immunotech). Serially diluted fusion proteins (R3.2x format HL822, SEQ ID NO: 10 and C-terminal format HL929, SEQ ID NO: 26) were then bound and detected with polyclonal goat anti-flagellin IgG (Covance Research Products). Plates were developed with rabbit anti-goat IgG conjugated to HRP (Jackson Immunoresearch), TMB and H₂SO₄. Curves were fit with a 4-parameter logistic equation (Softmax Pro 5.4).

FIG. 8 depicts in vitro Toll-like Receptor 5 (TLR5) assay for C. difficile toxin flagellin fusion proteins. Comparison of R3.2x formats for Toxins A and B. Serial dilutions of fusion proteins were mixed with HEK 293 cells (1×10⁴ per well) and incubated for 16-20 hours. Supernatant was removed and tested for the presence of IL-8 using an ELISA kit (BD Bioscience). OD values from the cell supernatants were converted to IL-8 concentration by interpolation from a standard curve run on the same plate. The influenza fusion protein R3.2x A/California/07/2009 (HL186, SEQ ID NO: 78) was employed as a control in the evaluated to fusion proteins that include Clostridium difficile antigens in the R3.2x formats. Toxin A antigen in the fusion protein referred to as HL821, SEQ ID NO: 9. Toxin B antigen in the fusion protein referred to as HL822, SEQ ID NO: 10.

FIG. 9 depicts in vitro TLR5 assay for C. difficile toxin flagellin fusion proteins. Comparison of C-terminal formats for Toxins A and B. Serial dilutions of fusion proteins are mixed with HEK 293 cells (about 1×10⁴ per well) and incubated for about 16 to about 20 hours. Supernatant was removed and tested for the presence of IL-8 using an ELISA kit (BD Bioscience). OD values from the cell supernatants were converted to IL-8 concentration by interpolation from a standard curve run on the same plate. The fusion proteins that include an influenza antigen A/California/07/2009 fused to the C-terminal of flagellin (referred to as HL184, SEQ ID NO: 79) were tested along with a fusion protein that included Toxin A fused to the C-terminal of flagellin (referred to as HL928, SEQ ID NO: 25) and fusion proteins that include Toxin B fused to the C-terminal of flagellin (referred to as HL929, SEQ ID NO: 26).

FIG. 10 depicts TLR5-Fc competition assay for C. difficile Toxin B/flagellin fusion proteins: comparison of R3.2x formats for Toxin A and B fusion proteins. Serial dilutions of R3.2x format fusion proteins (influenza R3.2x CA07 fusion protein control, SEQ ID NO: 78; Toxin A fusion protein referred to as HL821, SEQ ID NO: 9; Toxin B fusion protein referred to as HL822, SEQ ID NO: 10) and constant TLR5-Fc (R&D Systems) were preincubated for one hour. ELISA plates were coated with flagellin. Fusion proteins and TLR5-Fc mixes were added to flagellin-coated plates. Unbound TLR5-Fc was washed away and bound TLR5-Fc was detected with rabbit anti-mouse IgG conjugated to HRP (Jackson Immunoresearch), TMB and H₂SO₄. OD values were fit with a 4-parameter logistic equation (Softmax Pro 5.4).

FIG. 11 depicts TLR5-Fc competition assay for C. difficile Toxin B/flagellin fusion proteins: comparison of C-terminal formats for Toxin A and B fusion proteins. Serial dilutions of C-terminal format fusion proteins (influenza HA CA07 control: SEQ ID NO: 79, Toxin A: HL928, SEQ ID NO: 25; Toxin B: HL929, SEQ ID NO: 26) and constant TLR5-Fc (R and D Systems) were preincubated for one hour. ELISA plates were coated with flagellin. Fusion proteins and TLR5-Fc mixes were added to flagellin-coated plates. Unbound TLR5-Fc was washed away and bound TLR5-Fc was detected with rabbit anti-mouse IgG conjugated to HRP (Jackson Immunoresearch), TMB and H₂SO₄. OD values were fit with a 4-parameter logistic equation (Softmax Pro 5.4).

FIGS. 12A and 12B depict mouse serum ELISA for C. difficile Toxin/flagellin fusion proteins: post-prime: comparison of R3.2x and C-terminal formats. Serum was prepared from mice immunized with either the R3.2x format (Toxin A: HL821, SEQ ID NO: 9 or Toxin B:HL822, SEQ ID NO: 10), the C-terminal format (Toxin A: HL928, SEQ ID NO: 25 or Toxin B:HL929, SEQ ID NO: 26), Toxoid A or B in Montanide adjuvant (positive control) or buffer (F147) as a negative control. ELISA plates were coated with Toxoid A (FIG. 12A) or Toxoid B (FIG. 12B) (List Biological Laboratories). Serially diluted post-prime serum were then bound and detected with rabbit anti-mouse IgG conjugated to HRP (Jackson Immunoresearch), TMB and H₂SO₄. OD values were converted to specific IgG in μg/mL by comparison to a standard curve of polyclonal mouse IgG (AbD Serotec) fit with a 4-parameter logistic equation (Softmax Pro 5.4).

FIGS. 13A and 13B depict mouse serum ELISA for C. difficile Toxin/flagellin fusion proteins: post-boost 2: comparison of R3.2x and C-terminal formats. Serum was prepared from mice immunized with either the R3.2x format (Toxin A: referred to as HL821, SEQ ID NO: 9 or Toxin B: referred to as HL822, SEQ ID NO: 10), the C-terminal format (Toxin A: referred to as HL928, SEQ ID NO: 25 or Toxin B: referred to as HL929, SEQ ID NO: 26), Toxoid A or B in Montanide adjuvant (positive control) or buffer (F147) as a negative control. ELISA plates were coated with Toxoid A (FIG. 13A) or Toxoid B (FIG. 13 B) (List Biological Laboratories). Serially diluted post-boost 2 serum from mice were then bound and detected with rabbit anti-mouse IgG conjugated to HRP (Jackson Immunoresearch), TMB and H₂SO₄. OD values were converted to specific IgG in pg/mL by comparison to a standard curve of polyclonal mouse IgG (AbD Serotec) fit with a 4-parameter logistic equation (Softmax Pro 5.4).

FIGS. 14A, 14B and 14C depict mouse serum neutralization assay for C. difficile Toxin/flagellin fusion proteins: comparison of R3.2x and C-terminal formats. CT26 cells were treated with a cytostatic dose (about 100 ng/mL) of Toxin A (List Biological Laboratories) to block cell growth. After about 72 hours of Toxin A treatment, live cells were quantified using a luciferase assay (Cyto-Glo, Promega) to detect ATP released after cell lysis. Post-boost 2 serum from mice immunized with either R3.2x format (referred to as HL821, SEQ ID NO: 9, FIG. 14A) or C-terminal format (referred to as HL928, SEQ ID NO: 25) Toxin A fusion proteins were diluted 1:100; 1:316.2; 1:1,000 or 1:3,162 and incubated with the Toxin prior to adding to the cells. Serum from mice immunized with Toxoid A in Montanide adjuvant was included as a positive control, and serum from buffer (F147) treated mice served as a negative control. Data are presented as percent growth relative to control cells without Toxin A (100%) and cells with Toxin A, but no serum (0%). Points and lines represent results of individual mice (FIGS. 14A and 14B) or pools (FIG. 14C).

FIGS. 15A and B depict mouse serum neutralization assays for C. difficile Toxin/flagellin fusion proteins: testing of C-terminal format. CT26 cells were treated with a cytostatic dose (about 100 ng/mL) of Toxin B (List Biological Laboratories) to block cell growth. After about 72 hours of Toxin B treatment, live cells were quantified using a luciferase assay (Cyto-Glo, Promega) to detect ATP released after cell lysis. Post-boost 2 serum from mice immunized with the C-terminal format (referred to as HL929, SEQ ID NO: 26, FIG. 15A) Toxin B fusion protein were diluted 1:100; 1:316.2; 1:1,000; or 1:3,162 and incubated with the Toxin prior to adding to the cells. Pooled serum from mice immunized with Toxoid B in Montanide adjuvant was included as a positive control, and pooled serum from buffer (F147) treated mice served as a negative control (FIG. 15C). Data are presented as percent growth relative to controls cells without Toxin B (100%) and cells with Toxin B but no serum (0%). Points and lines represent results of individual mice (FIG. 15A) or pools (FIG. 15B).

### DETAILED DESCRIPTION OF THE INVENTION

The features and other details of the invention, either as steps of the invention or as combinations of parts of the invention, will now be more particularly described and pointed out in the claims. It will be understood that the particular embodiments of the invention are shown by way of illustration and not as limitations of the invention. The principle features of this invention can be employed in various embodiments without departing from the scope of the invention.

The risk of developing clinical disease and the risk of recurrent Clostridium difficile-associated diarrhea are increased in persons who do not mount a sufficient antibody response to toxins of Clostridium difficile bacteria, such as toxin A or toxin B. A threshold of greater than about 3 ELISA Units (EU) of anti-Toxin A IgG differentiated hospital patients who developed asymptomatic Clostridium difficile colonization from those that developed Clostridium difficile-associated diarrhea (Kyne, L., et al., N. Eng. J. Med., 342: 390-397 (2000)). Once infected, individuals who develop strong anti-toxin antibody responses clear their disease following antimicrobial treatment and remain disease free. Low levels of antibodies against toxin B receptor binding domain were related to recurrent disease. Circulating antibodies that neutralize Clostridium difficile toxin may reduce the risk of disease in humans.

In humans, a Clostridium difficile vaccine candidate is based on formalin-inactivated toxins A and B (toxoids) has been described (Foglia, G., et al. Vaccine, 30: 4307-4309 (2012), Greenberg, R. N., et al. Vaccine, 30: 2245-2249 (2012)). In a reported Phase I study, fifty healthy adult (18-55 years) and 48 elderly (≧65 years) volunteers were randomized to receive the toxoid-based vaccine (2 μg, 10 μg or 50 μg) or placebo on Days 0, 28, and 56. For toxin A, seroconversion by Day 56 (post-dose 2) was observed in 100% of volunteers aged 18-55 years in all dose groups and in 50%, 89%, and 100% of elderly participants in the 2 μg, 10 μg, and 50 μg dose groups, respectively. For both age groups, seroconversion for toxin B was suboptimal and lower than toxin A. Thus, sufficient potency is a concern with use of the toxoid vaccine and leads to the requirement of high doses of the toxoid for Toxin A in the key target population of the elderly. Even high doses are insufficient for Toxin B in the elderly. Additionally, in order to produce the toxins from the native organism, the cytoxicity of the purified toxins must be reduced by several orders of magnitude, typically by chemical inactivation, such as formaldehyde treatment. Large scale production of the highly toxic toxins followed by chemical inactivation is a significant production disadvantage to generate vaccines for Clostridium difficile.

Strategies to recombinantly produce Clostridium difficile toxins or fragments of the toxins in E. coli, yeast or baculovirus have been explored, but production levels of the toxins in these systems is often low, and/or insufficiently immunogenic unless delivered in combination with an exogenous adjuvant (Donald, R. G. K., et al., Microbiology 159:1254-1266 (2013), Ghose, C., et al., Infect. Immun. 81:2190-2196 (2013); Leuzzi, et al., Infection and Immunity 81:2851-2860 (2013)). For example, fusion of portions of Clostridium difficile antigens, such as the receptor binding domain, to flagellin that lacks domain 2 and domain 3 resulted in relatively low production when expressed recombinantly compared to constructs fused to portions of flagellin referred to as “R3′ constructs” or “R3 constructs” and flagellin that includes domains 2 and 3 (full-length flagellin), as depicted below.

| Construct | Yield |
|---|---|
| R3′.2x | 0.365† |
| STF2R3′f1.RBD-A1 | |
| (HL821, SEQ ID 9) | |
| C-terminal | 0.176† |
| STF2.RBD-A2 | |
| (HL928, SEQ ID 25) | |
| Delta | 0.044† |
| STF2Δ.RBD-A2 | |
| (HL1101, SEQ ID 27) | |
| R3′.2x | 0.139† |
| STF2R3′f1.RBD-B1 | |
| (HL822, SEQ ID 10) | |
| C-terminal | 0.121† |
| STF2.RBD-B2 | |
| (HL929, SEQ ID 26) | |
| Delta | 0.068† |
| STF2Δ.RBD-B2 | |
| (HL1102, SEQ ID 28) | |

†mg protein per gram of cell paste

The present invention has several advantages over currently available compositions that have production and potency challenges by fusing particular domains of toxin A, toxin B and/or binary toxin of Clostridium difficile to flagellin.

In a particular embodiment, the invention fuses portions of Clostridium difficile toxin A to the carboxy-terminal amino acid residue of flagellin to generate at least one first fusion protein and fuses portions of Clostridium difficile toxin B to the carboxy-terminal amino acid residue of flagellin to generate at least one second fusion protein. The first and second fusion proteins can be combined to form compositions for use to ameliorate Clostridium difficile associated disease.

Compositions that include a first fusion protein comprising at least a portion of Clostridium difficile toxin A fused to flagellin, in particular fused to the carboxy-terminal amino acid residue of flagellin or R3 flagellin constructs or R3′ flagellin constructs, and a second fusion protein comprising at least a portion of a Clostridium difficile toxin B fused to flagellin, in particular fused to the carboxy-terminal amino acid residue of flagellin or R3 flagellin constructs or R3′ flagellin constructs, can further include at least one additional fusion protein (a third fusion protein, a fourth fusion protein, a fifth fusion protein) comprising a flagellin, such as an R3 construct of flagellin or an R3′ construct of flagellin, or full length flagellin fused at the carboxy or amino terminus to at least one Clostridium difficile binary toxin antigen.

Toxins A and B generally have a relatively low pI, such as a pI of less than about 5.0 (about 5.0, about 4.5, about 4.0, about 3.5). The RBD of toxin A, which is a portion of toxin A, has a relatively high pI of greater than about 8.0 (about 8.0, about 8.5, about 9.0, about 9.5, about 10.0, or about 10.5). For example, the f1 fragment of toxin A has a pI of about 8.5 and RBD-A1 has a pI of about 8.5. The GTD of toxin A has a pI of about 5.5 and the CPD of toxin A has a pI of about 5.0. Toxin B antigens can have a relatively low pI of less than about 5.0 (about 5.0, about 4.5, about 4.0, about 3.5, about 3.0). For example, the f1 fragment of toxin B has a pI of about 4.0, the RBD-B2 of toxin B has a pI of about 4.0, the GTD of toxin B has a pI of about 4.5, the CPD of toxin B has a pI of about 4.5 and full length toxin B has a pI of about 4.5. Compositions that include at least one fusion protein having a toxin A antigen, in particular an RBD of toxin A, and at least one fusion protein having a toxin B antigen may result in a composition sufficiently balanced chemically to produce the desired immune response without unwanted side effects from, for example, excess TLR5 signaling from the flagellin component of the fusion protein.

The pI of the binary toxins vary. The full length cdtA has a pI of about 8.5 and the pI of cdtB has a pI of about 4.50 to about 4.9. Binary toxin antigens for use in the invention can varying depending on the binary toxin. Binary toxin antigens can be a relatively low pI, such as a pI of less than about 5.0 (about 5.0, about 4.5, about 4.0, about 3.5), or a relatively high pI of greater than about 8.0 (about 8.0, about 8.5, about 9.0, about 9.5, about 10.0, or about 10.5).

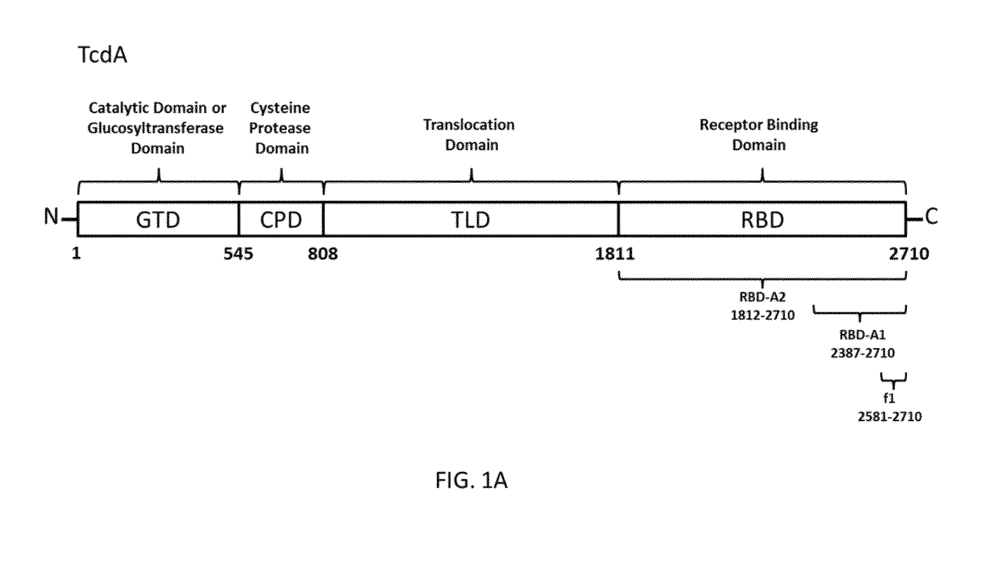

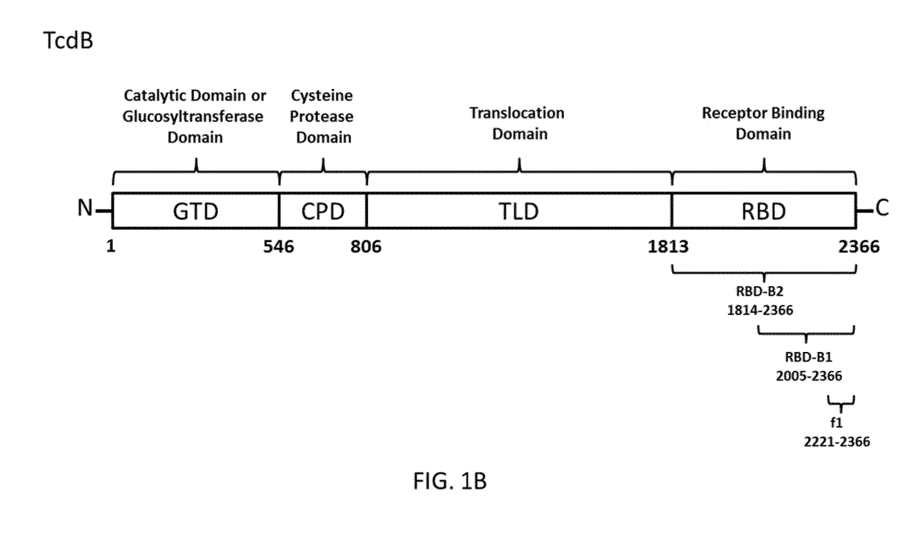

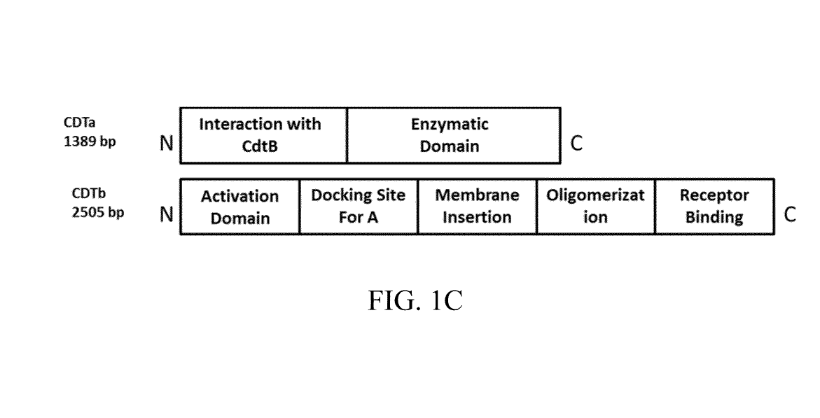

In another embodiment, compositions can further include a third fusion protein and a fourth fusion protein comprising a flagellin fused to at least one Clostridium difficile binary toxin. A Clostridium difficile binary toxin consists of two polypeptides, a Ctda polypeptide and a Ctdb polypeptides (FIG. 1C) (Barth, H., et al., Microbiol. Molecular Biol. Reviews 68:373-402 (2004)). The Ctda polypeptide (e.g., SEQ ID NO: 73) contains the ADP-ribosyltransferase (enzymatic domain) domain and a domain which interacts with the Ctdb chain (SEQ ID NO: 74), while the Ctdb polypeptide contains the receptor binding domain, an oligomerization domain, a membrane insertion domain, a Ctda interaction domain and an activation domain (FIG. 1C).

Binary toxin antigens for use in fusion proteins of the invention can include CdtA; the CdtB binding domain of CdtA; the enzymatic domain of CdtA; a portion or the entirety of CdtB; a portion or the entirety of the receptor binding domain of CdtB; or a portion or the entirety of the transmembrane pore forming domain of CdtB.

Generally, fusion of antigens to the carboxy-terminal amino acid residue of flagellin can result in a fusion protein that may be immunogenic, but has undesirable side effects, particular at doses sufficient to generate a suitable immune response. However, as described herein, fusion of Clostridium difficile antigens of particular domains of toxin A, toxin B and binary toxin to the carboxy-terminus of flagellin can result in a fusion protein that is sufficiently immunogenic with minimal side effects.

C. difficile produces two major virulence factors, TcdA and TcdB, which are large (about 270-about 308 kDa, ATCC 43255 strain), single-subunit toxins with multidomain structures that function as glucosyltransferases by inactivating GTPases within eukaryotic target cells. Both toxins are relatively large proteins, about 270-308 kDa, with domains depicted in FIGS. 1A and 1B. Each domain within the toxin corresponds to a function associated with the toxin. The N-terminal domain, which contains glucosyltransferase activity, is referred to as the catalytic or glucosyltransferase domain (GTD); the hydrophobic central region, important for translocating the toxins across the cell membrane, is referred to as the translocation domain (TLD); and the highly repetitive C-terminal domain, which appears to be primarily responsible for receptor binding, is referred to as the receptor binding domain (RBD).

In the initial step of toxin-mediated pathogenesis, the C-terminal region of TcdA or TcdB interacts with cell surface carbohydrate receptors. More specifically, the C-terminal receptor binding domain (RBD) binds carbohydrates on the surface of colonic epithelial cells as an initial step in pathogenesis. C. difficile toxins then enter the cell through receptor-mediated endocytosis, mediated by the translocation domain (TLD) and disrupt normal signaling pathways necessary for maintaining the cytoskeleton of the cell by the enzymatic domain (glucosyltransferase domain (GTD)), ultimately leading to inflammation and diarrhea.

The RBD is also called the C-terminal repetitive domain as predicted by sequence analysis and subsequently confirmed by crystal structures (f1) Ho, J. G., et al. PNAS, 20: 102(51): 18373-8 (2005), Greco, A., et al., Nat. Struct. Mol. Biol., 13(5): 460-461 (2006). Two types of repeats appear in both the sequence and the structure of this domain. One is a short repeat (SR) that contains about 15 to about 21 amino acid residues, typically including an amino acid (aa) pattern having a small side chain residue and a hydrophobic amino acid residue, followed by at least 7 hydrophobic amino acid residues and at least 3 aromatic amino acid residues and further followed by a loop of about 8 to about 9 amino acid residues. Examples of short repeats in the RBD are GFKIIDNKTYYYDEDSKLVK (SEQ ID NO: 70), GLININNSLFYFDPIEFNLVT (SEQ ID NO: 71), and GWQTINGKKYYFDINTGAALT (SEQ ID NO: 72), for example, in SEQ ID NOs: 25 and 26 short repeat fragments are shown with a single underline. The other repeat is a long repeat (LR) that includes about 30 amino acid residues, typically has a more conservative amino acid pattern of (Gly-Val-Phe-Xaa-Xaa-Xaa-Xaa-Gly-Phe(Tyr)-Glu-Tyr-Phe (SEQ ID NO:80) and a loop of about 19 amino acid residues. For example, in SEQ ID NOs: 25 and 26 long repeat fragments are shown with a double underline.

The crystal structure of Toxin A RBD (Ho, J. G., et al., PNAS, 20: 102(51): 18373-18378 (2005)) shows that each SR or LR contains a single β-hairpin consisting of a pair of five- to six-amino acid residue antiparallel β-strands connected by a tight turn (usually type I′) (Ho, J. G., et al., PNAS, 20: 102(51): 18373-18378 (2005). The structure of each β-hairpin is highly conserved, with the residues at positions 2 and 3 of strand 1 and positions 3, 4, and 5 of strand 2 of the first residue of the repeat forming a small hydrophobic cluster that brings consecutive pairs of β-hairpins together in a regularly repeating manner. Hydrophobic packing interactions or hydrogen-bonding interactions between adjacent pairs of β-hairpins dictate the regular arrangement of these secondary structural elements. Specifically, each adjacent pair of β-hairpins is related to the previous β-hairpin by a 3₁ screw-axis transformation, in which adjacent β-hairpins are related by a 120° rotation and a translation of about 10 Å, thus, creating a left-handed β-solenoid helix. This fold is predicted to be found in a wide range of bacterial cell-surface binding proteins and falls in the more general class of repeating solenoid fold proteins (Ho, J. G., et al., PNAS, 20: 102(51): 18373-18378 (2005). One carbohydrate binding site is seen by about 5 to about 7 tandem repeats that includes 1 LR (long term) repeat and several supporting SR (short term) repeats.

The two carbohydrate-binding sites in the TcdA-f2 structure (Greco, et al., Nat. Struct. Biol. 13:460-461 (2006)) are shallow troughs consisting of a LR and the hairpin turn of the following SR. The high level of sequence conservation in the LRs and key residues of the following SR suggests that the carbohydrate-binding mode seen in TcdA-f2 is conserved in all binding sites of C. difficile toxins. TcdA has about 32 SRs and about 7 LRs (that form 7 binding sites) and TcdB has about 19 SRs and 4LRs (that form 4 binding sites).

In the initial step of toxin-mediated pathogenesis, the C-terminal region of TcdA or TcdB interacts with cell surface carbohydrate receptors through the binding sites formed by the SRs and LRs of the RBD. The C-terminal and central regions of the toxin help mediate host cell entry by receptor mediated endocytosis. Once internalized, the toxin glucosylates Ras-like GTPases. The N-terminal glucosyltransferase domain (GTD) is a catalytic domain that glucosylates specific threonines in the host intestinal epithelial cells, leading to alterations in the actin cytoskeleton, massive fluid secretion, acute inflammation and necrosis of colonic mucosa. The cysteine protease domain (CPD) is a cellular inositol hexakisphosphate (InsP6) that induces an autocatalytic cleavage of the toxins, releasing the N-terminal glucosyltransferase domain (GTD) into the host cell cytosol. The cysteine protease domain (CPD) is responsible for autoprocessing within toxin.

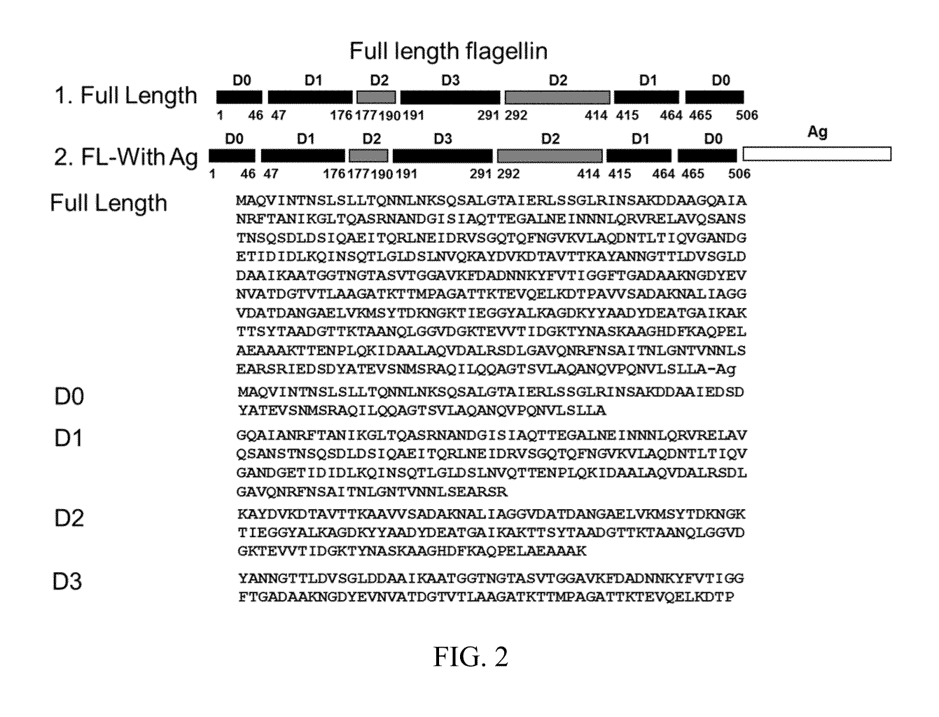

These structural domains or portions of domains of Clostridium difficile may be fused to portions or full length flagellin to generate fusion proteins of the invention that can be employed in the methods of the invention. Naturally occurring flagellin has an amino domain 0, an amino domain 1, an amino domain 2, a domain 3, a carboxy domain 2, a carboxy domain and a carboxy domain 0, as depicted, for example, in FIG. 2 (SEQ ID NO: 54).

The invention is generally directed to fusion proteins that include flagellin and one or more Clostridium difficile antigens, compositions that include fusion proteins of the invention and methods of treating humans with at least one fusion protein that includes at least one flagellin and one or more Clostridium difficile antigens.

“Fusion protein,” as used herein, refers to a protein that is generated by the joining of two components (also referred to herein as “fused” or linked”) (e.g., flagellin that activates a TLR5 and at least a portion of at least one Clostridium difficile antigen). Fusion proteins of the invention can be generated by recombinant DNA technologies or by chemical conjugation of the components of the fusion protein. Recombinant DNA technologies and chemical conjugation techniques are well established procedures and known to one of skill in the art. Exemplary techniques to generate fusion proteins that include Toll-like Receptor agonists are described herein and in U.S. application Ser. Nos. 11/714,684 and 11/714,873.

In an embodiment, the invention is a composition comprising a first fusion protein that activates Toll-like Receptor 5 and includes flagellin fused to at least one Clostridium difficile toxin A antigen; and a second fusion protein that activates Toll-like Receptor 5 and includes flagellin fused to at least one Clostridium difficile toxin B antigen. The first fusion protein can include a first Clostridium difficile toxin A antigen that replaces at least a portion of or the entirety of domain 3 of flagellin and a second Clostridium difficile toxin A antigen that is fused to the carboxy-terminal amino acid of flagellin; and the Clostridium difficile toxin B antigen can be fused to the carboxy-terminal amino acid of flagellin of the second fusion protein. In yet another embodiment, the first Clostridium difficile toxin A antigen is at least a portion of a receptor binding domain of Clostridium difficile toxin A and the second Clostridium difficile toxin A antigen is at least a portion, such as an f1 fragment, or the entirety of the receptor binding domain of Clostridium difficile toxin A.

In a further embodiment, the first fusion protein can include a first Clostridium difficile toxin A antigen that replaces at least a portion of or the entirety of domain 3 of flagellin and a second Clostridium difficile toxin A antigen that is fused to the carboxy-terminal amino acid of flagellin; and the second fusion protein can include a first Clostridium difficile toxin B antigen that replaces at least a portion of or the entirety of domain 3 of flagellin and a second Clostridium difficile toxin B antigen that is fused to the carboxy-terminal amino acid of flagellin. The first Clostridium difficile toxin B antigen can be at least a portion of a receptor binding domain of Clostridium difficile toxin B and the second Clostridium difficile toxin B antigen is at least a portion of an enzymatic domain (a glucosyltransferase domain) of Clostridium difficile toxin B.

The enzymatic domain of Clostridium difficile toxins can be employed in fusion proteins of the invention. The enzymatic domain of toxin A and toxin B is the glucosyltransferase domain. The enzymatic domain of a binary toxin is the ADP-ribosyltransferase domain.

In yet another embodiment, the invention is a composition comprising a first fusion protein that activates Toll-like Receptor 5 and includes flagellin fused to at least one Clostridium difficile toxin A antigen; a second fusion protein that activates Toll-like Receptor 5 and includes flagellin fused to at least one Clostridium difficile toxin B antigen; and at least one additional fusion protein (e.g., a third fusion protein and/or a fourth fusion protein) that comprises flagellin fused to at least one Clostridium difficile binary toxin antigen.

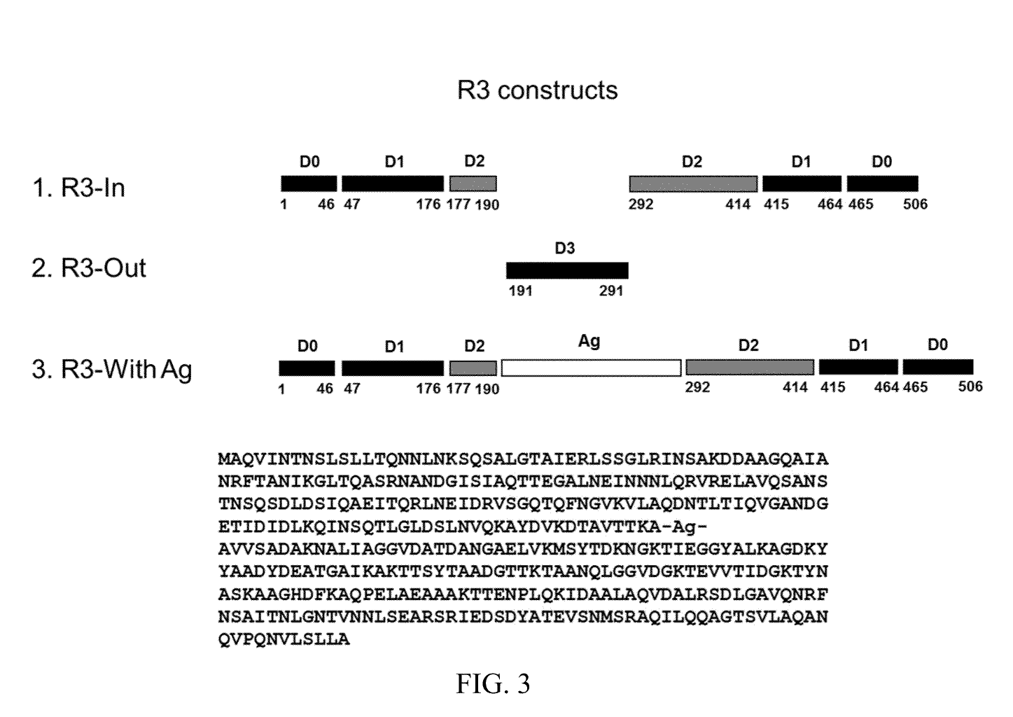

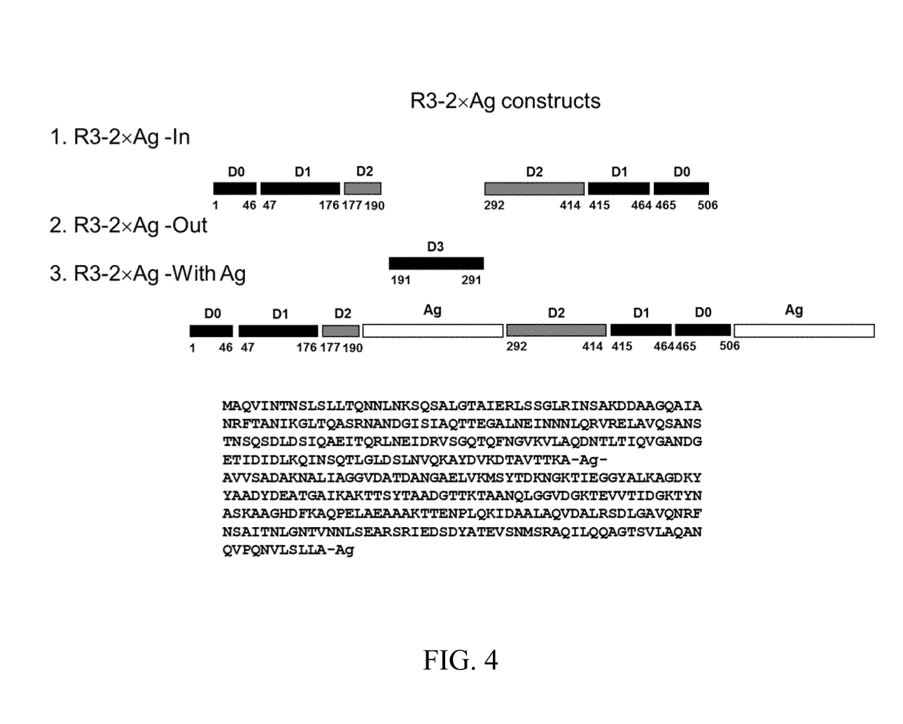

In an embodiment, the invention is a fusion protein comprising a flagellin protein in which domain 3 of the flagellin protein has been replaced with a first Clostridium difficile antigen and a second Clostridium difficile antigen is fused to the carboxy-terminal amino acid of the flagellin protein, wherein the fusion protein activates Toll-like Receptor 5. Flagellin in which domain 3 has been replaced with at least one Clostridium difficile antigen is referred to herein as an “R3 construct,” or Replacement of domain 3 constructs (FIG. 3), or “R3 format.” Flagellin in which domain 3 has been replaced with at least one Clostridium difficile antigen and at least one additional Clostridium difficile antigen has also been fused to the carboxy-terminal amino acid of the flagellin in which domain 3 has been replaced with an antigen is referred to as an “R32x” construct or Replacement of domain 3 constructs with 2 antigens (FIG. 4) or an “R32x format.” The generation of flagellin for R3 constructs and R32x constructs are described, for example, in U.S. Patent Application publication No: 2011/0135680 and WO 2009/128950. R3 constructs and R32x constructs of flagellin can be fused to at least one antigen described herein.

Portions of flagellin that lack at least a portion of domain 3 are referred to herein as “R3′ constructs” or “R3′ flagellin.” For example, an R3′ construct can be a flagellin in which amino acid residues 194-285 of SEQ ID NO: 81 have been deleted, which results in a portion of flagellin with 3 amino acid residues at the amino-terminal boundary of D3 and 6 amino acid residues at the carboxy-terminal boundary of D3; or amino acid residues 194-337 of SEQ ID NO: 82 have been deleted, which results in a portion of flagellin with 3 amino acid residues at the amino-terminal boundary of D3 and 6 amino acid residues at the carboxy-terminal boundary of D3; or amino acid residues 181-185 of SEQ ID NO: 83 have been deleted, which results in a portion of flagellin with 3 amino acid residues at the amino-terminal boundary of D3 and 6 amino acid residues at the carboxy-terminal boundary of D3. The amino acid residues that remain in domain 3 of flagellin can function as a natural linker for insertion of the Clostridium difficile antigen.

R3′ constructs of flagellin can be fused to a first Clostridium difficile antigen that replaces the portion of D3 that has been removed from the flagellin to generate R3′ fusion proteins. R3′ constructs of flagellin can be fused to a first Clostridium difficile antigen that replaces the portion of D3 that has been removed from the flagellin and a second Clostridium difficile antigen can be fused to the amino-terminal or carboxy-terminal amino acid of the R3′ construct to generate R3′2x fusion proteins.

Domains of exemplary flagellins for use in the invention are depicted in Table 1.

TABLE 1
Domains of Exemplary Flagellins for Use
in Fusion Proteins of the Invention

| | SEQ ID | Domain Boundary | | | | | | |
|---|---|---|---|---|---|---|---|---|
| Flagellin | Number | D0N | D1N | D2N | D3 | D2C | D1C | D0C |
| S. | 81 | 1- | 47- | 177- | 191- | 292- | 415- | 465- |
| typhimurium | | 46 | 176 | 190 | 291 | 414 | 464 | 506 |
| flijB | | | | | | | | |
| E. coli | 82 | 1- | 47- | 177- | 191- | 344- | 503- | 553- |
| | | 46 | 176 | 190 | 343 | 502 | 552 | 595 |
| B. subtilis | 83 | 1- | 45- | 171- | 178- | 192- | 236- | 286- |
| | | 44 | 177 | 177 | 191 | 235 | 286 | 317 |

In an embodiment, the invention is a fusion protein comprising at least one amino acid sequence as set forth in SEQ ID NO: 55 (an R3 construct) and at least a portion of at least one Clostridium difficile antigen, wherein the Clostridium difficile antigen is inserted between amino acid residues 190 and 191 of SEQ ID NO: 55, and wherein the fusion protein activates a Toll-like Receptor 5. Exemplary R3 constructs (also referred to herein as “R3 flagellin constructs” or “R3 form of flagellin”) can be fused to, for example, a Clostridium difficile antigen. In another embodiment, the portion of flagellin employed in fusion proteins and methods of the invention is flagellin lacking domain 3, for example, SEQ ID NO: 55, to which at least one Clostridium difficile antigen is fused at the carboxy-terminus or the amino-terminus of the portion of flagellin lacking domain 3. Exemplary fusion proteins that include an R3 construct include SEQ ID NOs: 13 and 14.

FIG. 3 depicts the domains (DO, Dl, D2, D3) of a flagellin construct (SEQ ID NO: 55) and a fusion protein that includes, in sequence, the amino-domain 0, the amino-domain 1, the amino-domain 2, the carboxy-domain 2, the carboxy-domain 1 and the carboxy-domain 0 of flagellin. A Clostridium difficile antigen (Ag) is fused between the amino- and carboxy-domain 2 of the flagellin construct. The flagellin construct of the fusion protein depicted in FIG. 3 lacks the D3 domain of flagellin and is referred to herein as an “R3 construct.” An amino acid sequence that activates a TLR5 and has at least about 50.0%, at least about 60.0%, at least about 80.0%, at least about 85.0%, at least about 90.0%, at least about 95.0%, at least about 98.0% and at least about 99.0% identity to the contiguous amino acid sequence as set forth in SEQ ID NO: 55 can be employed in the fusion proteins of the invention.

In an embodiment, the invention is a fusion protein that activates a Toll-like Receptor 5 that includes an R32x construct of flagellin, and at least two Clostridium difficile antigens. In an embodiment, at least one Clostridium difficile antigen is between amino acid residues 190 and 191 of SEQ ID NO: 54 and at least one other antigen is fused to amino acid residue 405 of SEQ ID NO: 54. Exemplary fusion proteins that include the R32x flagellin construct and Clostridium difficile antigens are SEQ ID NOs: 3, 4, 9 and 10.

Fusion of a Clostridium difficile antigen to the carboxy-terminus of flagellin, either full length flagellin or a portion of flagellin, such as flagellin lacking domain 3, can be fusion to the terminal (last) amino acid residue of the carboxy-domain 0 of a flagellin or to a terminal amino acid residue of flagellin that has less than the entirety of the carboxy-domain 0. “Less than the entirety,” with reference to a domain of flagellin, means about 60% to about 70% of the total number of amino acid residues that make up domain 0 of the flagellin. For example, with reference to Table 1, less than the entirety of the carboxy-domain 0 of S. typhimurium flijB can be amino acid residues 465-480 or 465-496 or 465-480 of SEQ ID NO: 81; less than the entirety of the carboxy-domain 0 of E. coli flagellin can be amino acid residues 553-565 or 553-575 or 553-585 of SEQ ID NO: 82; and less than the entirety of the carboxy-domain 0 of B. sublitis flagellin can be 286-310 or 286-297 of SEQ ID NO: 83.

In another embodiment, at least one Clostridium difficile antigen can be fused to the amino-terminus of flagellin. Fusion of at least one Clostridium difficile antigen to the amino-terminus of flagellin, either full length flagellin or a portion of flagellin, such as flagellin lacking domain 3, can be fusion to the terminal (first) amino acid residue of the amino-domain 0 of flagellin or to a first amino acid residue of flagellin that has less than the entirety of the amino-domain 0. For example, with reference to Table 1, less than the entirety of amino-domain 0 of S. typhimurium fljB flagellin can be amino acid residues 1-30, 10-46, or 15-46 of SEQ ID NO: 81; less than the entirety of amino-domain 0 of E. coli flagellin can be amino acid residues 1-30 or 10-46 or 15-46 of SEQ ID NO: 82; and less than the entirety of amino-domain 0 of B. subtilis flagellin can be 1-30 or 10-44 or 15-44 of SEQ ID NO: 83.

The flagellin in the compositions, fusion proteins and methods described herein can be at least a portion of the S. typhimurium flagellin selected from the group consisting of SEQ ID NO: 54, SEQ ID NO: 55, SEQ ID NO: 81; at least a portion of an S. muenchen flagellin (GenBank Accession Number AB028476) that includes at least a portion of SEQ ID NO: 84 and SEQ ID NO: 85; at least a portion of P. aeruginosa flagellin that includes at least a portion of SEQ ID NO: 86; at least a portion of a Listeria monocytogenes flagellin that includes at least a portion of SEQ ID NO: 87; at least a portion of an E. coli flagellin that includes at least a portion of SEQ ID NO: 88 and SEQ ID NO: 89; at least a portion of a Yersinia flagellin; and at least a portion of a Campylobacter flagellin.

Fusion proteins of the invention can be made employing routine recombinant molecular biological techniques. The host cells employed to make the fusion proteins of the invention can be a prokaryotic host cell or a eukaryotic host cell. The prokaryotic host cell can be at least one member selected from the group consisting of an E. coli prokaryotic host cell, a Pseudomonas prokaryotic host cell, a Bacillus prokaryotic host cell, a Salmonella prokaryotic host cell and a P. fluorescens prokaryotic host cell.

Constructs encoding fusion proteins of the invention can be cloned into pET-24a expression vectors. Competent BLR (DE3) E. coli cell lines are transformed and target protein expression is confirmed by SDS-PAGE and Western blot using toxin specific antibodies (Meridian). C-terminal fusion (C-term) constructs partition to the soluble fraction and R3.2x constructs partition into inclusion bodies, regardless of the properties of the vaccine antigen. Fermentation is carried out in shake flasks (1-10L) or bioreactors (≦12L). Expression is induced with 1 mM IPTG. After induction, cells are harvested and lysed using a homogenizer. Soluble protein from constructs is purified by ion exchange and size exclusion chromatography. Insoluble protein from constructs including protein from inclusion bodies is recovered by centrifugation and washed using detergent containing wash buffers to remove endotoxin, nucleic acids and other impurities. Inclusion bodies are re-solubilized in 8M urea and protein refolded by rapid dilution into buffer without urea. Refolded protein is further purified by ion exchange chromatography and size exclusion chromatography. Final protein concentration is determined by the Micro-BCA Kit (Pierce Biotechnology). A target purity of greater than about 90% should be achievable.

Eukaryotic host cells can be employed to make the fusion proteins of the invention can include a Saccharomyces eukaryotic host cell, an insect eukaryotic host cell (e.g., at least one member selected from the group consisting of a Baculovirus infected insect cell, such as Spodoptera frugiperda (Sf9) or Trichhoplusia ni (High5) cells; and a Drosophila insect cell, such as Dme12 cells), a fungal eukaryotic host cell, a parasite eukaryotic host cell (e.g., a Leishmania tarentolae eukaryotic host cell), CHO cells, yeast cells (e.g., Pichia) and a Kluyveromyces lactis host cell.

Suitable eukaryotic host cells and vectors can also include plant cells (e.g., tomato; chloroplast; mono- and dicotyledonous plant cells; Arabidopsis thaliana; Hordeum vulgare; Zea mays; potato, such as Solanum tuberosum; carrot, such as Daucus carota L.; and tobacco, such as Nicotiana tabacum, Nicotiana benthamiana (Gils, M., et al., Plant Biotechnol J. 3: 613-20 (2005); He, D. M., et al., Colloids Surf B Biointerfaces, (2006); Huang, Z., et al., Vaccine 19: 2163-71 (2001); Khandelwal, A., et al., Virology, 308: 207-15 (2003); Marquet-Blouin, E., et al., Plant Mol Biol, 51: 459-69 (2003); Sudarshana, M. R., et al. Plant Biotechnol J., 4: 551-9 (2006); Varsani, A., et al., Virus Res, 120: 91-6 (2006); Kamarajugadda S., et al., Expert Rev Vaccines, 5: 839-49 (2006); Koya V, et al., Infect Immun. 73: 8266-74 (2005); Zhang, X., et al., Plant Biotechnol J., 4: 419-32 (2006)).

A “first Clostridium difficile antigen” or a “second Clostridium difficile antigen,” as used herein, means any portion or the entirety of a Clostridium difficile toxin A, toxin B or a binary toxin antigen. For example, a first or a second Clostridium difficile antigen can be a portion or the entirety of the glucosyltransferase domain, a portion or the entirety of the cysteine protease domain, a portion or the entirety of the translocation domain or a portion or the entirety of the receptor binding domain, or combinations thereof The first and the second Clostridium difficile antigen can be the same or distinct Clostridium difficile antigens. For example, a Clostridium difficile antigen of GTD is distinct from a Clostridium difficile CPD antigen.

A “portion” of a Clostridium difficile antigen would be less than the entirety of a Clostridium difficile antigen, in particular, a toxin A, toxin B or a binary toxin antigen. The Clostridium difficile antigens can be portions of the GTD (e.g., SEQ ID NOs: 57, 64), CPD (e.g., SEQ ID NOs: 59, 66), TD or RBD (e.g., SEQ ID NOs: 61, 62, 68, 69) domains of the toxin A, B or binary toxin that would be sufficient to generate an antibody response against a Clostridium difficile toxin. In an embodiment the antibody response generated to the Clostridium difficile antigen would neutralize a Clostridium difficile toxin.

Neutralizing titers in serum can be measured using a cell-based assay. The enzymatic activity of toxin A or B results in changes in cell morpohology and blockage of cell growth. Preincubation of immune serum with toxins allows measurement of neutralizing titers. Monoclonal antibodies CDA1 and CDB1 were found to prevent disease reoccurrence at about 10 to about 100 μg/mL. These antibodies are neutralizing in a cell-based assays at about 15 ng/mL for toxin A and about 45 ng/mL for toxin B. Dividing the clinically effective dose by the neutralizing concentration, titers of 667 to 6,667 for toxin A and 222 to 2,222 for toxin B would be expected to be neutralizing titers of antibody (Lowy, I., et al., N. Engl. J. Med. 362:197-205 (2010), Babcock, G. J., et al., Infect. Immun. 74:6339-6347 (2006)).

In an embodiment, fusion proteins of the invention that include a toxin A antigen generate a neutralizing antibody titer of at least about 1/600 (about 1/650, about 1/700, about 1/750, about 1/800, about 1/850, about 1/900, about 1/950, about 1/1000). In another embodiment, fusion proteins of the invention that include a toxin B antigen generate a neutralizing antibody titer of at least about 1/200 (about 1/225, about 1/250, about 1/300, about 1/350, about 1/400, about 1/500).

Neutralizing activity can also be detected in serum using cellular cytotoxicity assays. For example, cells, such as IMR-90 or CT26 cells, are exposed to Toxin A or B for about 18 hours in culture. As a result of the enzymatic activity of the toxins, cell morphology changes from adherent to rounded. This can be scored using a microscope and the toxin concentration at which 50% of the cells are rounded estimated (Babcock, G. J., et al., Infect. Immunol. 74:6339-6347 (2006)). Alternatively, an automated cell scoring system can be employed (Xie, J., et al., Clin. Vaccine Immunol., 20: 517-525 (2013)). Using a fixed amount of toxin, serum from immunized animals can be titrated and an inhibitory mid-points (IC₅₀) determined. Cells can also be incubated with either Toxin A or B for about 48 to about 72 hours resulting in an arrest of cell growth. Live cells can be quantified by measuring ATP with a luciferase assay (Donald, R. G. K., et al., Microbiology 159:1254-1266 (2013)). Addition of a cell lysis agent, luciferase and luciferin results in chemiluminescence proportional to the ATP in live cells. The light can be measured by a 96 well luminescent plate reader. As with the cell rounding assay, with a fixed concentration of toxin, serum from immunized animals can be titrated and an inhibitory mid-points (IC₅₀) determined.

Portions of flagellin employed in the fusion proteins of the invention can include less than the entirety of a naturally occurring flagellin, such as flagellin that lacks a domain 3, referred to as “R3 constructs” of flagellin depicted in FIG. 3, as described herein.

In an embodiment, the R3 construct of flagellin is fused to a first Clostridium difficile antigen by replacing domain 3 of flagellin with the first Clostridium difficile antigen, which results in fusion of the Clostridium difficile antigen between the amino domain 2 of flagellin and the carboxyl domain 2 of flagellin.

“Activates Toll-like Receptor 5 (TLR5)” when referring to a fusion protein of the invention, means that the fusion protein stimulates a response associated with TLR5, for example, host inflammatory responses (Smith, K. D., et al., Nature Immunology 4:1247-1253 (2003)), such as Interleuken-8 (IL-8) production, tumor necrosis factor (TNF) production and NK-κB activation, as described herein. Techniques to assess TLR5 activation are well-established and are described herein. For example, techniques to assess activation of a TLR5 include use of a commercially available cell line, HEK 293 cells, by measuring induction of IL-8 production when the HEK 293 cells are cultured with fusion proteins of the invention. The cultured HEK 293 cells are exposed to fusion proteins of the invention after which the supernatants are evaluated for IL-8 by a commercially available ELISA kit, as described herein.

The first Clostridium difficile antigen and the second Clostridium difficile antigen of the fusion proteins of the invention can include a Clostridium difficile antigen that is a toxin A antigen, a toxin B antigen, a binary toxin antigen, or any combination thereof. Toxin A and toxin B antigens for use in the fusion proteins described herein, can include at least a portion or the entirety of the glucosyltransferase domain (GTD) (also referred to herein “catalytic domain”), a portion or the entirety of cysteine protease domain (CPD), a portion of the entirety translocation domain (TLD) and a portion of the entirety of a receptor binding domain (RBD).

The domains of Toxin A of Clostridium difficile (TcdA) are depicted in FIG. 1A. In an embodiment, when a portion or the entirety of the glucosyltransferase domain of Clostridium difficile toxin A or toxin B is employed in the fusion proteins of the invention, the amino acids responsible for the enzymatic activity of the glucosyltransferase domain are inactivated. In particular, the DxD motif of the GTD of toxin A (amino acid residues 2-545 of SEQ ID NO: 15) can have mutations D269N (aspartic acid at position 269 to asparagine) and D285N (aspartic acid at position 285 to asparagine) to yield SEQ ID NO: 57. Likewise, the GTD of toxin B (amino acid residues 2-546 of SEQ ID NO: 16) can have point mutations D270N and D286N to yield SEQ ID NO: 64.

Exemplary domains of toxin A include the glucosyltransferase domain of amino acid residues 1-545 of SEQ ID NO: 15 (e.g., SEQ ID NO: 56), a cysteine protease domain of amino acid residues 546-808 of SEQ ID NO: 15 (e.g., SEQ ID NO: 58), a translocation domain of amino acid residues 809-1811 of SEQ ID NO: 15 and a receptor binding domain of amino acid residues 1812-2710 of SEQ ID NO: 15 (e.g., SEQ ID NO: 60). The receptor binding domain of a Clostridium difficile toxin, for example, Toxin A, can be further subdivided into a receptor binding domain A1 (RBD-A1), which is a portion of the receptor binding domain, for example, as depicted in FIG. 1A of amino acid residues 2387-2710 of SEQ ID NO: 15 (SEQ ID NO 61). Receptor binding domain A1, includes an additional portion referred to as the “f1 fragment”. The f1 fragment of a Clostridium difficile toxin is a portion of the receptor binding domain that extends to the most carboxy-terminal amino acid residue. An exemplary f1 fragment of toxin A is amino acid residues 2581-2710 of SEQ ID NO: 15 (SEQ ID NO: 90). The crystal structure of the receptor-binding domain C-terminal repeats for Clostridium difficile toxin A has been described, for example, by Ho, J. G. S., et al., PNAS, 102: 18373-18378 (2005). The f1 fragment is highly repetitive.

A schematic depiction of the glucosyltransferase, cysteine protease, translocation and receptor binding domains of a Clostridium difficile toxin B are depicted in FIG. 1B. Domains of toxins B include, a glucosyltransferase domain of amino acid residues 1-546 of SEQ ID NO: 16 (e.g., SEQ ID NO: 63), a cysteine protease domain of amino acid residues 547-806 of SEQ ID NO: 16 (e.g., SEQ ID NO: 65), a translocation domain of amino acid residues 807-1813 of SEQ ID NO: 16 and a receptor binding domain of amino acid residues 1814-2366 of SEQ ID NO: 16 (e.g., SEQ ID NO: 67). Similar to toxin A, the receptor binding domain of Clostridium difficile toxin B includes a RBD-B l region, which is depicted, for example, as amino acid residues 2005-2366 of SEQ ID NO: 16 (SEQ ID NO: 68). The f1 fragment of an exemplary Clostridium difficile toxin B receptor binding domain is amino acid residues 2221-2366 of SEQ ID NO: 16 (SEQ ID NO: 91).

In an embodiment, the first Clostridium difficile antigen that replaces domain 3 of the flagellin protein in the fusion proteins of the invention, is a portion of the receptor binding domain of Clostridium difficile toxin A and the second Clostridium difficile antigen that is fused to the carboxy-terminal amino acid of the flagellin protein is a portion or the entirety of the receptor binding domain of Clostridium difficile toxin A. In a particular embodiment, the first Clostridium difficile antigen that replaces domain 3 of the flagellin protein of the fusion protein of the invention, is a portion of a receptor binding domain of Clostridium difficile A, such as an f1 fragment of the RDB (e.g., SEQ ID NO: 91), and the second Clostridium difficile antigen fused to the carboxy-terminal amino acid of the flagellin protein in the fusion proteins of the invention is the entirety of the receptor binding domain of Clostridium difficile toxin A (e.g., SEQ ID NO: 60). In another embodiment, the first Clostridium difficile antigen that replaces domain 3 of the flagellin protein of the fusion protein of the invention, is a portion of a receptor binding domain of Clostridium difficile A, such as an f1 fragment of the RDB (e.g., SEQ ID NO: 62), and the second Clostridium difficile antigen fused to the carboxy-terminal amino acid of the flagellin protein in the fusion proteins of the invention is a portion of the receptor binding domain of Clostridium difficile toxin A (e.g., SEQ ID NO: 61) to yield an R32x fusion protein (SEQ ID NO: 9).

In another embodiment, the first Clostridium difficile antigen that replaces the domain 3 of a flagellin protein of the fusion proteins of the invention is a Toxin B antigen and the second Clostridium antigen that is fused to the carboxy-terminal amino acid of the flagellin protein and fusion protein is a Clostridium difficile toxin B antigen.

Fusion proteins that include Clostridium difficile toxin B antigens, can include a first Clostridium difficile antigen that is a portion of a receptor binding domain of Clostridium difficile toxin B antigen that replaces domain 3 of the flagellin protein and the second Clostridium difficile antigen can be a portion or the entirety of the glucosyltransferase domain of the Clostridium difficile toxin B antigen.

In a particular embodiment, the fusion proteins of the invention include a first Clostridium difficile antigen of toxin B that is a portion of the receptor binding domain of Clostridium difficile toxin B fused to flagellin with a second Clostridium difficile antigen that is the entirety of the glucosyltransferase domain of toxin B fused to the carboxy-terminal amino acid of the fusion protein. In an embodiment, the portion of the first Clostridium difficile antigen that replaces domain 3 of the flagellin protein is an f1 portion of a receptor binding domain of Clostridium difficile toxin B, such as SEQ ID NO: 69 to yield an R32x fusion protein (SEQ ID NO: 10).

In a further embodiment, the fusion protein of the invention includes a first Clostridium difficile antigen that is a first portion of a receptor binding domain of Clostridium difficile toxin B that replaces domain 3 of a flagellin protein and a second Clostridium difficile antigen is a second portion or the entirety of the receptor binding domain of Clostridium difficile toxin B fused to the carboxy-terminal amino acid of the flagellin protein. In an embodiment, the portion of the receptor binding domain is an f1 portion of the receptor binding domain, such as SEQ ID NO: 69. In another embodiment, the portion of the receptor binding domain of Clostridium difficile toxin B is SEQ ID NO: 67 or SEQ ID NO: 68.

The first and second portions of the RBD employed in fusion proteins of the invention can be the same portions of the RBD or distinct portions of the RBD.

In a further embodiment, the first Clostridium difficile antigen that replaces domain 3 of a flagellin protein in the fusion proteins of the invention and the second Clostridium difficile antigen that is fused to the carboxy-terminal amino acid residue of the flagellin protein in which domain 3 has been replaced with the first Clostridium difficile antigen, can include a portion or the entirety of the translocation domain of a Clostridium difficile toxin A or a Clostridium difficile toxin B.

In yet another embodiment, the first Clostridium difficile antigen that replaces domain 3 of flagellin and the second Clostridium difficile antigen that is fused to the carboxy-terminal amino acid of the flagellin in which domain 3 has been replaced with a first Clostridium difficile antigen can be a portion or the entirety of a Clostridium difficile binary toxin.

In a further embodiment, fusion proteins of the invention can include a portion or the entirety of any combination of the glucosyltransferase domain, cysteine protease domain, translocation domain and receptor binding domain of a Clostridium difficile toxin A, Clostridium difficile toxin B or Clostridium difficile binary toxin.

In an embodiment, the fusion proteins of the invention can include a first Clostridium difficile toxin A antigen that replaces domain 3 of flagellin and a second Clostridium difficile toxin B antigen fused to the carboxy-terminal amino acid residue of the flagellin. For example, the f1 domain of the RBD of toxin A can be engineered to replace at least a portion or the entirety of domain 3 of flagellin and the GTD of toxin B can be fused to the carboxy-terminal amino acid of an R3 or R3′ construct.

In another embodiment, the fusion proteins of the invention can include a first Clostridium difficile toxin B antigen that replaces domain 3 of flagellin and a second Clostridium difficile toxin A antigen fused to the carboxy-terminal amino acid residue of flagellin. For example, an f1 fragment of the RBD domain can be engineered to replace at least a portion of or the entirety D3 in the R3 or R3′ constructs and at least a portion or the entirety of the GTD can be fused to the carboxy-terminal amino acid of the R3 or R3′ construct.

In a further embodiment, an embodiment of the invention is a fusion protein comprising a full-length flagellin or a portion of flagellin, such as an R3′ construct, fused at the carboxy-terminal amino acid to at least one Clostridium difficile antigen selected from the group consisting of a portion or the entirety of a receptor binding domain of Clostridium difficile, a portion or the entirety of a translocation domain of Clostridium difficile and a portion or the entirety of a glucosyltransferase domain.

In still another embodiment, the invention is a fusion protein comprising a full length flagellin or a portion of flagellin, such as an R3 construct, fused at the amino-terminal amino acid to at least one Clostridium difficile antigen selected from the group consisting of a portion or the entirety of a receptor binding domain of Clostridium difficile, a portion or the entirety of a translocation domain of Clostridium difficile and a portion of the entirety of a glucosyltransferase domain.

In an embodiment, the full-length flagellin or portions of flagellin of fusion proteins of the invention, are fused to a portion or the entirety of the receptor binding domain of Clostridium difficile toxin A. In another embodiment, the full-length flagellin or portions of flagellin of the fusion proteins of the invention, are fused, in sequence, to a portion or the entirety of the translocation domain of Clostridium difficile toxin B and a portion or the entirety of the receptor binding domain of Clostridium difficile toxin B. In another embodiment, the full-length flagellin or portions of flagellin of the fusion proteins of the invention are fused to a portion or the entirety of the receptor binding domain of Clostridium difficile toxin B. In yet another embodiment, the full-length flagellin or portions of flagellin of the fusion proteins of the invention are fused to a portion or the entirety of the receptor binding domain of a binary toxin of Clostridium difficile. In a further embodiment, any combination of portions of the domains of Clostridium difficile toxin A, toxin B and binary toxin are fused to full-length flagellin or a portion of flagellin at the carboxy terminus.

In yet another embodiment, the invention is a fusion protein comprising a portion of flagellin fused at the carboxy-terminal amino acid to at least one Clostridium difficile antigen selected from the group consisting of a portion or the entirety of a receptor binding domain of Clostridium difficile, a portion or the entirety of a translocation domain of Clostridium difficile and a portion or the entirety of a glucosyltransferase domain of Clostridium difficile, wherein the portion of the flagellin is not a portion of flagellin lacking the entirety of amino domain 2, domain 3 and a carboxy domain 2.

Fusion proteins of the invention, described herein, can be employed in the compositions administered to humans by the methods described herein.

In yet another embodiment, the invention is a method of treating a human, comprising a step of administering to the human at least one a fusion protein that includes a full-length flagellin protein fused at the carboxy-terminal amino acid to at least one Clostridium difficile antigen selected from the group consisting of a portion or the entirety of a receptor binding domain of Clostridium difficile, a portion or the entirety of a translocation domain of Clostridium difficile and a portion or the entirety of a glucosyltransferase domain. Administration of the composition that includes Clostridium difficile antigen fused to full-length flagellin ameliorates a Clostridium difficile associated disease. For example, amelioration of the Clostridium difficile associated disease can be a consequence of the human generating neutralizing antibodies to at least one of the Clostridium difficile antigen employed and fused to the full-length flagellin.

“Clostridium difficile associated disease,” as used herein, refers to an interruption, cessation or disorder of a bodily function or system as a consequence of the secretion of toxin by Clostridium difficile bacteria. Primary risk factors for Clostridium difficile associated disease are exposure to antibiotics, exposure to healthcare environment, such as hospitalization, and acid suppressing medications. Clostridium difficile associated disease can include antibiotic associated diarrhea, pseudomembranous colitis which can progress to toxic megacolon that can be life threatening.

Techniques to assess whether or not a neutralizing antibody has been generated in the human following the administration of a composition that includes fusion proteins of the invention, are well established and known to one of ordinary skill in the art. For example, a mouse model can be used to determine if the immune responses are protective against Clostridium difficile infection, that the antibodies are neutralizing antibodies to Clostridium difficile antigen (Chen, X., et al., Gastroenterology, 135: 1984-1992 (2008)). Aggressive treatment of standard lab mice with a range of antibiotics followed by clindamycin, disturbs the normal gut flora and allows the development of a lethal infection with Clostridium difficile (strain VPI 10463, ATCC 43255) that resembles human disease by both symptoms and histology. Notably, mice that survive initial infection are resistant to reinfection, indicating that the model is suitable for evaluating fusion proteins of the invention.

Fusion proteins can be assessed for neutralization ability by injection into C57BL/6 mice in a series of doses to generate immune serum. Serum is evaluated for neutralization titers using an in vitro assay described by Babcock et al., Infection and Immunity, 74: 6339-6347 (2006)). Serum is diluted, mixed with purified toxin A or B and incubated for one hour. The mixture is added to cultured IMR-90 or CT26 cells and incubated for an additional 18 to 72 hours. Alternatively, cell viability can be assessed at about 72 hours by measuring ATP released after adding a lysis reagent in the presence of luciferin, as described herein. Cytotoxic activity of the residual toxin A or B can be assessed by microscopic evaluation of cell rounding at 18 hours on a scale of 0-4. The dilution of serum that produces 50% cytotoxicity is the value to compare candidates. Serum from mice immunized with different candidates may be mixed in order to determine if neutralization is additive or synergistic. Optimal doses and interval to achieve high neutralizing titers and the strength of response within 7 days of priming is determined. The serum from these studies is tested in the neutralization assays. Optimal doses and interval to achieve high neutralizing titers is determined. Also strength of response within 7-14 days of the last dose is determined.

Fusion proteins can be further evaluated by immunizing C57BL/6 mice with a range of doses. Following immunization, mice will be treated with antibiotics and clindamycin, followed by Clostridium difficile challenge. The dose of Clostridium difficile will be an LD₉₀ dose. Mice will be monitored for survival and weight loss, as well as symptoms, such as ruffled fur, diarrhea, and reduced activity. A standard 0-4 symptom scale will be used for the subjective scores.

In an embodiment, the invention is a method of treating a human comprising the step of administering to the human at least one a fusion protein that activates a Toll-like Receptor 5 and includes a flagellin protein in which domain 3 of the flagellin protein has been replaced with a first Clostridium difficile antigen and a second Clostridium difficile antigen is fused to a carboxy-terminal amino acid of the flagellin protein. Administration of the fusion protein that activates Toll-like Receptor 5 that includes a first Clostridium difficile antigen replacing domain 3 of flagellin and a second Clostridium difficile antigen fused to the most carboxy amino acid of the flagellin, can ameliorate a Clostridium difficile associated disease. Amelioration of disease can be generation of neutralizing antibodies in the human to at least one of the first or the second Clostridium difficile antigen or both the first and second Clostridium difficile antigen.

In a further embodiment, the invention is a method of treating a human, comprising administering to the human at least one fusion protein that includes a portion of flagellin fused at the carboxy-terminal amino acid to at least one Clostridium difficile antigen selected from the group consisting of a portion or the entirety of a receptor binding domain of Clostridium difficile, and a portion or the entirely of a translocation domain of Clostridium difficile and a portion or the entirety of a glucosyltransferase domain of Clostridium difficile, wherein the portion of the flagellin is not a portion of flagellin lacking all of an amino domain 2, domain 3 and a carboxy domain 2, and whereby administration of the fusion protein ameliorates a Clostridium difficile associated disease. Amelioration of the Clostridium difficile associated disease can be by generating neutralizing antibodies to at least one of the Clostridium difficile antigens.

In an additional embodiment, the invention is a method of treating a human, comprising administering to the human at least one fusion protein that includes a portion of flagellin fused at the carboxy-terminal amino acid to at least one Clostridium difficile antigen selected from the group consisting of a portion or the entirety of a translocation domain of Clostridium difficile and a portion or the entirety of a glucosyltransferase domain of Clostridium difficile, whereby administration of the fusion protein ameliorates a Clostridium difficile associated disease. In embodiments, the portion of flagellin can be an R3 construct (flagellin lacking a domain 3) or flagellin lacking both domain 2 and domain 3, referred to herein as “STFΔ.”

In an embodiment, methods of the invention that ameliorate Clostridium difficile associated disease include a composition comprising (1) a fusion protein of flagellin and a Clostridium difficile toxin A antigen as described herein; (2) a fusion protein of flagellin and a Clostridium difficile toxin B antigen as described herein; (3) a fusion protein of flagellin and a Clostridium difficile binary toxin A antigen as described herein; and (4) a fusion protein of flagellin and a Clostridium difficile binary toxin B antigen as described herein.

The fusion proteins comprising a flagellin and at least one Clostridium difficile antigen can further include a linker between at least one component of the fusion protein (flagellin) and at least one other component of the fusion protein (Clostridium difficile antigen) or any combination thereof. “Linker,” as used herein in reference to a fusion protein, means a connector between components of the fusion protein in a manner that the components are not directly joined. Fusion proteins can include a combination of linkers between distinct components of the fusion protein to similar or like components of the fusion protein. The linker can be an amino acid linker that can includes naturally occurring or synthetic amino acid residues. The amino acid linker can be of varying lengths. In an embodiment, the amino acid linker can include multiple, consecutive glycine residues, such as GGGG (SEQ ID NO: 92) or GGGGG (SEQ ID NO: 93), which are employed in fusion proteins of SEQ ID NOs: 62, 69 or other suitable amino acids that have few or no side chains, including multiple, consecutive serine residues, such as SSSS (SEQ ID NO: 94) or SSSSS (SEQ ID NO: 95), which are considered flexible linkers.

Exemplary fusion proteins of the invention can include SEQ ID NOs: 1-10, 13, 14, 25, 26, 29 and 30.

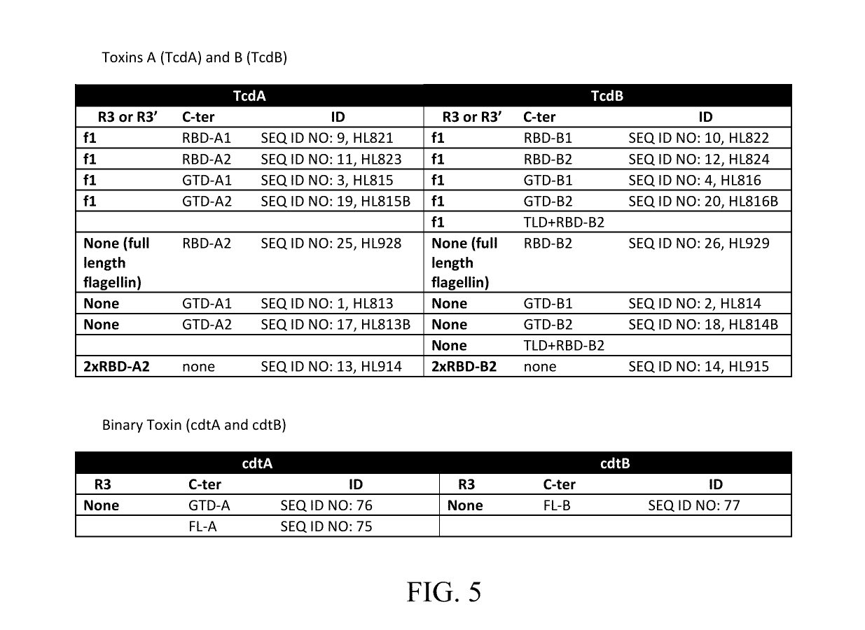

Exemplary fusion proteins that include Clostridium difficile toxin A (TcdA) antigens and Clostridium difficile toxin B (TcdB) antigens are also depicted in FIG. 5. “R3” refers to replacement of domain 3 of flagellin. RDB refers to the receptor binding domain of the Clostridium difficile toxin. GTD refers to the glucosyltransferase domain of the Clostridium difficile toxin. (See FIGS. 1A and 1B). When full length flagellin is employed in the fusion protein “R3” is indicated as none with mutation of “full length flagellin.”

The human administered fusion proteins of the invention, and the methods described herein, can be a human that is at least 49 years old. In another embodiment, the human administered a fusion protein of the invention can be at least about 50 years old, about 64 years old, at least about 65 years old. In further embodiments, the methods of the invention can employ fusion proteins of the invention to treat a human that is about 1 years old, about 2 years old, about 3 years old, about 4 years old, about 5 years old, or about 6 years old. In an additional embodiment, the human treated by the methods of the invention with the fusion proteins of the invention can be between about 13 years old and about 18 years old. In another embodiment, the human treated by the methods of the invention with the fusion proteins of the invention can be a peripartum woman.

In a particular embodiment, the Clostridium difficile associated disease that is minimized following administration of fusion proteins of the invention is an antibiotic-associated diarrhea. In another embodiment, the Clostridium difficile associated disease that is minimized following administration of the fusion proteins of the invention is a pseudomembranous colitis.

The fusion proteins of the invention employed in the methods of the invention can be administered to the human following association with a virosome or a virus-like particle.

The dose of the fusion protein may be administered to the human within a range of doses including from about 0.1 μg to about 500 μg, 1 μg to about 100 μg, 1 μg to about 50 μg, from about 1 μg to about 30 μg, from about 1 μg to about 25 μg, from about 1 μg to about 20 μg, from about 1 μg to about 15 μg, from about 1 μg to about 10 μg, from about 2 μg to about 50 μg, 2 μg to about 30 μg, from about 2 μg to about 20 μg, from about 2 μg to about 10 μg, from about 2 μg to about 8 μg, from about 3 μg to about 50 μg, 3 μg to about 30 μg, from about 3 μg to about 20 μg, from about 3 μg to about 10 μg, from about 3 μg to about 8 μg, from about 3 μg to about 5 μg, from about 4 μg to about 50 μg, 4 μg to about 30 μg, from about 4 μg to about 20 μg, from about 4 μg to about 10 μg, from about 4 μg to about 8 μg, from about 5 μg to about 50 μg, 5 μg to about 30 μg, from about 5 μg to about 20 μg, from about 5 μg to about 10 μg, from about 5 μg to about 9 μg, and from about 5 μg to about 8 μg.

In an embodiment, the fusion proteins of the invention employed in the methods of the invention are administered to a human at a dose of at least one member selected from the group consisting of 0.5 μg, 1.0 μg, 1.5 μg, 5 μg, 10 μg, 20 μg, 40 μg and 80 μg.

The fusion protein for use according to the present invention may be delivered as a standard 0.5 ml injectable dose and contain from about 0.1 μg to about 50 μg of antigen. In a preferred embodiment, the fusion protein for use in the methods of the invention can be a standard 0.5 ml injectable dose and that contains from about 3 μg to about 20 μg of fusion protein in a composition of between about 0.25 and about 1.0 ml, suitably between about 0.5 ml and about 1.0 ml, in particular a standard about 0.5 ml. Low volume doses of compositions that include fusion proteins of the invention can be below about 0.5 ml, typically below about 0.3 ml and usually not less than about 0.1 ml.

An “effective amount” when referring to the amount of a composition and a fusion protein administered to the human, refers to that amount or dose of the composition that, when administered to the subject is an amount sufficient for therapeutic efficacy (e.g., an amount sufficient to stimulate an immune response in a subject, an amount sufficient to ameliorate Clostridium difficile associated disease).

The methods of the present invention can be accomplished by the administration of the compositions and fusion proteins of the invention by enteral or parenteral means. Specifically, the route of administration is by intramuscular injection of the composition and fusion protein. Other routes of administration are also encompassed by the present invention including intravenous, intradermal, interarterial, interperitoneal, intranasal, transdermal, suppositories or subcutaneous routes.

The compositions that include the fusion proteins can be administered alone or as admixtures with conventional excipients, for example, pharmaceutically, or physiologically, acceptable organic, or inorganic carrier substances suitable for enteral or parenteral application which do not deleteriously react with the composition. Suitable pharmaceutically acceptable carriers include water, salt solutions (such as Ringer's solution), alcohols, oils, gelatins and carbohydrates such as lactose, amylose or starch, fatty acid esters, hydroxymethylcellulose, and polyvinyl pyrolidine. Such preparations can be sterilized and, if desired, mixed with auxiliary agents such as lubricants, preservatives, stabilizers, wetting agents, emulsifiers, salts for influencing osmotic pressure, buffers, coloring and/or aromatic substances and the like which do not deleteriously react with the compositions administered to the human.

The compositions, fusion proteins and proteins of the invention can be administered to a subject on a support that presents the compositions, proteins and fusion proteins of the invention to the immune system of the subject to generate an immune response in the subject. The presentation of the compositions, proteins and fusion proteins of the invention would preferably include exposure of antigenic portions of the fusion protein to generate antibodies. The support is biocompatible. “Biocompatible” as used herein, means that the support does not generate an immune response in the subject (e.g., the production of antibodies).

The dosage and frequency (single or multiple doses) administered to a subject can vary depending upon a variety of factors, including, for example, prior exposure to an infection consequent to exposure to the antigen: health, body weight, body mass index, and diet of the subject or health-related problems. Other therapeutic regimens or agents can be used in conjunction with the methods and compositions, proteins or polypeptides of the present invention, including the use of adjuvants.

The composition can be administered to the human in a single dose or in multiple doses, such as at least two doses. When multiple doses are administered to the subject, a second or third dose can be administered days (e.g., 1, 2, 3, 4, 5, 6, 7), weeks (e.g., 1, 2, 3, 4, 5, 6, 7, 8, 9, 10), months (e.g., 1, 2, 3, 4, 5, 6, 7, 8, 9, 10) or years (e.g., 1, 2, 3, 4, 5, 6, 7, 8, 9, 10) after the initial dose. For example, a second dose of the composition can be administered about 7 days, about 14 days or about 28 days following administration of a first dose of the composition that includes the fusion protein.

Exemplary fusion proteins and Clostridium difficile antigens (also referred to as “C. dif. antigen”) for use in fusion proteins of the invention include: SEQ ID NO: 1, STF2.GTD-A1 (C-terminal fusion with Clostridium difficile Toxin A (TcdA) N-terminal Glucosyltransferase Domain (GTD), D269N+D285N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLASLISKEELIKLAYSIRPRENEYKTILTNLDEYNKLTTNNNENKY
LQLKKLNESIDVFMNKYKTSSRNRALSNLKKDILKEVILIKNSNTSPVEK
NLHFVWIGGEVSDIALEYIKQWADINAEYNIKLWYDSEAFLVNTLKKAIV
ESSTTEALQLLEEEIQNPQFDNMKFYKKRMEFIYDRQKRFINYYKSQINK
PTVPTIDDIIKSHLVSEYNRDETVLESYRTNSLRKINSNHGIDIRANSLF
TEQELLNIYSQELLNRGNLAAASNIVRLLALKNFGGVYLNVDMLPGIHSD
LFKTISRPSSIGLDRWEMIKLEAIMKYKKYINNYTSENFDKLDQQLKDNF
KLIIESKSEKSEIFSKLENLNVSDLEIKIAFALGSVINQALISKQGSYLT
NLVIEQVKNRYQFLNQHLNPAIESDNNFTDTTKIFHDSLFNSATAENSMF
LTKIAPYLQVGFMPEARSTISLSGPGAYASAYYDFINLQENTIEKTLKAS
DLIEFKFPENNLSQLTEQEINSLWSFDQASAKYQFEKYVRDYTGGSLSED

SEQ ID NO: 2, STF2.GTD-B1 (C-terminal fusion with Clostridium difficile Toxin B (TcdB) GTD, D270N+D286N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLASLVNRKQLEKMANVRFRTQEDEYVAILDALEEYHNMSENTVVEK
YLKLKDINSLTDIYIDTYKKSGRNKALKKFKEYLVTEVLELKNNNLTPVE
KNLHFVWIGGQINDTAINYINQWKDVNSDYNVNVFYDSNAFLINTLKKTV
VESAINDTLESFRENLNDPRFDYNKFFRKRMEIIYDKQKNFINYYKAQRE
ENPELIIDDIVKTYLSNEYSKEIDELNTYIEESLNKITQNSGNDVRNFEE
FKNGESFNLYEQELVERWNLAAASNILRISALKEIGGMYLNVDMLPGIQP
DLFESIEKPSSVTVDFWEMTKLEAIMKYKEYIPEYTSEHFDMLDEEVQSS
FESVLASKSDKSEIFSSLGDMEASPLEVKIAFNSKGIINQGLISVKDSYC
SNLIVKQIENRYKILNNSLNPAISEDNDFNTTTNTFIDSIMAEANADNGR
FMMELGKYLRVGFFPDVKTTINLSGPEAYAAAYQDLLMFKEGSMNIHLIE
ADLRNFEISKTNISQSTEQEMASLWSFDDARAKAQFEEYKRNYFEGSLGE
D

SEQ ID NO: 3, STF2R3′(f1).GTD-A1 (The f1 fragment of TcdA Receptor Binding Domain (RBD) replaced D3 of flagellin with single underlined flexible linkers and C-terminal fusion with TcdA GTD, D269N+D285N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLASLISKEELIKLAYSIRPRENEYKTILTNLDEYNKLTTNNNENKYLQL
KKLNESIDVFMNKYKTSSRNRALSNLKKDILKEVILIKNSNTSPVEKNLH
FVWIGGEVSDIALEYIKQWADINAEYNIKLWYDSEAFLVNTLKKAIVESS
TTEALQLLEEEIQNPQFDNMKFYKKRMEFIYDRQKRFINYYKSQINKPTV
PTIDDIIKSHLVSEYNRDETVLESYRTNSLRKINSNHGIDIRANSLFTEQ
ELLNIYSQELLNRGNLAAASNIVRLLALKNFGGVYLNVDMLPGIHSDLFK
TISRPSSIGLDRWEMIKLEAIMKYKKYINNYTSENFDKLDQQLKDNFKLI
IESKSEKSEIFSKLENLNVSDLEIKIAFALGSVINQALISKQGSYLTNLV
IEQVKNRYQFLNQHLNPAIESDNNFTDTTKIFHDSLFNSATAENSMFLTK
IAPYLQVGFMPEARSTISLSGPGAYASAYYDFINLQENTIEKTLKASDLI
EFKFPENNLSQLTEQEINSLWSFDQASAKYQFEKYVRDYTGGSLSED

SEQ ID NO: 4, STF2R3′(f1).GTD-B1 (The f1 fragment of TcdB RBD replaced D3 of flagellin with single underlined flexible linkers and C-terminal fusion with TcdA GTD, D270N+D286N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGKY
YFNPETKKACKGINLIDDIKYYFDEKGIMRTGLISFENNNYYFNENGEMQ
FGYINIEDKMFYFGEDGVMQIGVFNTPDGFKYFAHQNTLDENFEGESINY
TGWLDLDEKRYYFTDEYIAATGSVIIDGEEYYFDPDTAQLVISEGGGGEL
KDTPAVVSADAKNALIAGGVDATDANGAELVKMSYTDKNGKTIEGGYALK
AGDKYYAADYDEATGAIKAKTTSYTAADGTTKTAANQLGGVDGKTEVVTI
DGKTYNASKAAGHDFKAQPELAEAAAKTTENPLQKIDAALAQVDALRSDL
GAVQNRFNSAITNLGNTVNNLSEARSRIEDSDYATEVSNMSRAQILQQAG
TSVLAQANQVPQNVLSLLASLVNRKQLEKMANVRFRTQEDEYVAILDALE
EYHNMSENTVVEKYLKLKDINSLTDIYIDTYKKSGRNKALKKFKEYLVTE
VLELKNNNLTPVEKNLHFVWIGGQINDTAINYINQWKDVNSDYNVNVFYD
SNAFLINTLKKTVVESAINDTLESFRENLNDPRFDYNKFFRKRMEIIYDK
QKNFINYYKAQREENPELIIDDIVKTYLSNEYSKEIDELNTYIEESLNKI
TQNSGNDVRNFEEFKNGESFNLYEQELVERWNLAAASNILRISALKEIGG
MYLNVDMLPGIQPDLFESIEKPSSVTVDFWEMTKLEAIMKYKEYIPEYTS
EHFDMLDEEVQSSFESVLASKSDKSEIFSSLGDMEASPLEVKIAFNSKGI
INQGLISVKDSYCSNLIVKQIENRYKILNNSLNPAISEDNDFNTTTNTFI
DSIMAEANADNGRFMMELGKYLRVGFFPDVKTTINLSGPEAYAAAYQDLL
MFKEGSMNIHLIEADLRNFEISKTNISQSTEQEMASLWSFDDARAKAQFE
EYKRNYFEGSLGED

SEQ ID NO: 5, STF2.CPD-A1 (C-terminal fusion with TcdA Cysteine Protease Domain (CPD), D589N+H655A+C700S to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLASEDNGVDFNKNTALDKNYLLNNKIPSNNVEEAGSKNYVHYIIQL
QGNDISYEATCNLFSKNPKNSIIIQRNMNESAKSYFLSDDGESILELNKY
RIPERLKNKEKVKVTFIGAGKDEFNTSEFARLSVDSLSNEISSFLDTIKL
DISPKNVEVNLLGSNMFSYDFNVEETYPGKLLLSIMDKITSTLPDVNKNS
ITIGANQYEVRINSEGRKELLAHSGKWINKEEAIMSDLSSKEYIFFDSID
NKLKAKSKNIPGLASISEDIKT

SEQ ID NO: 6, STF2.CPD-B1 (C-terminal fusion with TcdB Cysteine Protease Domain, D587N+H653A+C698S to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLAGEDDNLDFSQNIVVDKEYLLEKISSLARSSERGYIHYIVQLQGN
KISYEAACNLFAKTPYDSVLFQKNIEDSEIAYYYNPGDGEIQEIDKYKIP
SIISDRPKIKLTFIGAGKDEFNTDIFAGFDVDSLSTEIEAAIDLAKEDIS
PKSIEINLLGSNMFSYSINVEETYPGKLLLKVKDKISELMPSISQDSIIV
SANQYEVRINSEGRRELLDHSGEWINKEESIIKDISSKEYISFNPKENKI
TVKSKNLPELSTLLQEIRN

SEQ ID NO: 7, STF2R3′(f1).CPD-A1 (The f1 fragment of TcdA RBD replaced D3 of flagellin with single underlined flexible linkers and C-terminal fusion with TcdA CPD, D589N+H655A+C700S to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLASEDNGVDFNKNTALDKNYLLNNKIPSNNVEEAGSKNYVHYIIQLQGN
DISYEATCNLFSKNPKNSIIIQRNMNESAKSYFLSDDGESILELNKYRIP
ERLKNKEKVKVTFIGAGKDEFNTSEFARLSVDSLSNEISSFLDTIKLDIS
PKNVEVNLLGSNMFSYDFNVEETYPGKLLLSIMDKITSTLPDVNKNSITI
GANQYEVRINSEGRKELLAHSGKWINKEEAIMSDLSSKEYIFFDSIDNKL
KAKSKNIPGLASISEDIKT

SEQ ID NO: 8, STF2R3′(f1).CPD-B1 (The f1 fragment of TcdB RBD replaced D3 of flagellin with single underlined flexible linkers and C-terminal fusion with TcdB CPD, D587N+H653A+C698S to abolish enzymatic activity; C. diff. antigen is double underlined)

ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGKY
YFNPETKKACKGINLIDDIKYYFDEKGIMRTGLISFENNNYYFNENGEMQ
FGYINIEDKMFYFGEDGVMQIGVFNTPDGFKYFAHQNTLDENFEGESINY
TGWLDLDEKRYYFTDEYIAATGSVIIDGEEYYFDPDTAQLVISEGGGGEL
KDTPAVVSADAKNALIAGGVDATDANGAELVKMSYTDKNGKTIEGGYALK
AGDKYYAADYDEATGAIKAKTTSYTAADGTTKTAANQLGGVDGKTEVVTI
DGKTYNASKAAGHDFKAQPELAEAAAKTTENPLQKIDAALAQVDALRSDL
GAVQNRFNSAITNLGNTVNNLSEARSRIEDSDYATEVSNMSRAQILQQAG
TSVLAQANQVPQNVLSLLAGEDDNLDFSQNIVVDKEYLLEKISSLARSSE
RGYIHYIVQLQGNKISYEAACNLFAKTPYDSVLFQKNIEDSEIAYYYNPG
DGEIQEIDKYKIPSIISDRPKIKLTFIGAGKDEFNTDIFAGFDVDSLSTE
IEAAIDLAKEDISPKSIEINLLGSNMFSYSINVEETYPGKLLLKVKDKIS
ELMPSISQDSIIVSANQYEVRINSEGRRELLDHSGEWINKEESIIKDISS
KEYISFNPKENKITVKSKNLPELSTLLQEIRN

SEQ ID NO: 9, HL821, STF2R3′(f1).RBD-A1 (The f1 fragment of TcdA Receptor Binding Domain (RBD) replaced D3 of flagellin with single underlined flexible linkers and C-terminal fusion with 14 repeats of the C-terminal TcdA of C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLAASTGYTSINGKHFYFNTDGIMQIGVFKGPNGFEYFAPANTDANNIEG
QAILYQNKFLTLNGKKYYFGSDSKAVTGLRTIDGKKYYFNTNTAVAVTGW
QTINGKKYYFNTNTSIASTGYTIISGKHFYFNTDGIMQIGVFKGPDGFEY
FAPANTDANNIEGQAIRYQNRFLYLHDNIYYFGNNSKAATGWVTIDGNRY
YFEPNTAMGANGYKTIDNKNFYFRNGLPQIGVFKGSNGFEYFAPANTDAN
NIEGQAIRYQNRFLHLLGKIYYFGNNSKAVTGWQTINGKVYYFMPDTAMA
AAGGLFEIDGVIYFFGVDGVKAPG

SEQ ID NO: 10, HL822, STF2R3′(f1).RBD-B1 (The f1 fragment of TcdB RBD replaced D3 of flagellin with single underlined flexible linkers and C-terminal fusion with 16 repeats of the C-terminal TcdB of C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGKY
YFNPETKKACKGINLIDDIKYYFDEKGIMRTGLISFENNNYYFNENGEMQ
FGYINIEDKMFYFGEDGVMQIGVFNTPDGFKYFAHQNTLDENFEGESINY
TGWLDLDEKRYYFTDEYIAATGSVIIDGEEYYFDPDTAQLVISEGGGGEL
KDTPAVVSADAKNALIAGGVDATDANGAELVKMSYTDKNGKTIEGGYALK
AGDKYYAADYDEATGAIKAKTTSYTAADGTTKTAANQLGGVDGKTEVVTI
DGKTYNASKAAGHDFKAQPELAEAAAKTTENPLQKIDAALAQVDALRSDL
GAVQNRFNSAITNLGNTVNNLSEARSRIEDSDYATEVSNMSRAQILQQAG
TSVLAQANQVPQNVLSLLAMKVGYTEIDGKHFYFAENGEMQIGVFNTEDG
FKYFAHHNEDLGNEEGEEISYSGILNFNNKIYYFDDSFTAVVGWKDLEDG
SKYYFDEDTAEAYIGLSLINDGQYYFNDDGIMQVGFVTINDKVFYFSDSG
IIESGVQNIDDNYFYIDDNGIVQIGVFDTSDGYKYFAPANTVNDNIYGQA
VEYSGLVRVGEDVYYFGETYTIETGWIYDMENESDKYYFNPETKKACKGI
NLIDDIKYYFDEKGIMRTGLISFENNNYYFNENGEMQFGYINIEDKMFYF
GEDGVMQIGVFNTPDGFKYFAHQNTLDENFEGESINYTGWLDLDEKRYYF
TDEYIAATGSVIIDGEEYYFDPDTAQLVISE

SEQ ID NO: 11, STF2R3′(f1).RBD-A2 (The f1 fragment of TcdA RBD replaced D3 of flagellin with single underlined flexible linkers and C-terminal fusion with full receptor binding domain with residues 1812-2710 of TcdA; 40 repeats of C. diff. antigen in italics. Note: Single underline, short repeat; double underline, long repeat)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLAGFKIIDNKTYYYDEDSKLVKGLININNSLFYFDPIEFNLVTGWQTIN
GKKYYFDINTGAALTSYKIINGKHFYFNNDGVMQLGVFKGPDGFEYFAPA
NTQNNNIEGQAIVYQSKFLTLNGKKYYFDNNSKAVTGWRIINNEKYYFNP
NNAIAAVGLQVIDNNKYYFNPDTAIISKGWQTVNGSRYYFDTPTAIAFNG
YKTIDGKHFYFDSDCVVKIGVFSTSNGFEYFAPANTYNNNIEGQAIVYQS
KFLTLNGKKYYFDNNSKAVTGWQTIDSKKYYFNTNTAEAATGWQTIDGKK
YYFNTNTAEAATGWQTIDGKKYYFNTNTAIASTGYTIINGKHFYFNTDGI
MQIGVFKGPNGFEYFAPANTDANNIEGQAILYQNEFLTLNGKKYYFGSDS
KAVTGWRIINNKKYYFNPNNAIAAIHLCTINNDKYYFSYDGILQNGYITI
ERNNFYFDANNESKMVTGVFKGPNGFEYFAPANTHNNNIEGQAIVYQNKF
LTLNGKKYYFDNDSKAVTGWQTIDGKKYYFNLNTAEAATGWQTIDGKKYY
FNLNTAEAATGWQTIDGKKYYFNTNTFIASTGYTSINGKHFYFNTDGIMQ
IGVFKGPNGFEYFAPANTDANNIEGQAILYQNKFLTLNGKKYYFGSDSKA
VTGLRTIDGKKYYFNTNTAVAVTGWQTINGKKYYFNTNTSIASTGYTIIS
GKHFYFNTDGIMQIGVFKGPDGFEYFAPANTDANNIEGQAIRYQNRFLYL
HDNIYYFGNNSKAATGWVTIDGNRYYFEPNTAMGANGYKTIDNKNFYFRN
GLPQIGVFKGSNGFEYFAPANTDANNIEGQAIRYQNRFLHLLGKIYYFGN
NSKAVTGWQTINGKVYYFMPDTAMAAAGGLFEIDGVIYFFGVDGVKAPGI
YG

SEQ ID NO: 12, STF2R3′(f1).RBD-B2 (The f1 fragment of TcdB RBD replaced D3 of flagellin with single underlined flexible linkers and C-terminal fusion with TcdB, full receptor binding residues 1814-2366, 24 repeats of C. diff. antigen in italics. Note: Single underline, short repeat; double underline, long repeat)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGKY
YFNPETKKACKGINLIDDIKYYFDEKGIMRTGLISFENNNYYFNENGEMQ
FGYINIEDKMFYFGEDGVMQIGVFNTPDGFKYFAHQNTLDENFEGESINY
TGWLDLDEKRYYFTDEYIAATGSVIIDGEEYYFDPDTAQLVISEGGGGEL
KDTPAVVSADAKNALIAGGVDATDANGAELVKMSYTDKNGKTIEGGYALK
AGDKYYAADYDEATGAIKAKTTSYTAADGTTKTAANQLGGVDGKTEVVTI
DGKTYNASKAAGHDFKAQPELAEAAAKTTENPLQKIDAALAQVDALRSDL
GAVQNRFNSAITNLGNTVNNLSEARSRIEDSDYATEVSNMSRAQILQQAG
TSVLAQANQVPQNVLSLLAGLVSLYNEKFYINNFGMMVSGLIYINDSLYY
FKPPVNNLITGFVTVGDDKYYFNPINGGAASIGETIIDDKNYYFNQSGVL
QTGVFSTEDGFKYFAPANTLDENLEGEAIDFTGKLIIDENIYYFDDNYRG
AVEWKELDGEMHYFSPETGKAFKGLNQIGDYKYYFNSDGVMQKGFVSIND
NKHYFDDSGVMKVGYTEIDGKHFYFAENGEMQIGVFNTEDGFKYFAHHNE
DLGNEEGEEISYSGILNFNNKIYYFDDSFTAVVGWKDLEDGSKYYFDEDT
AEAYIGLSLINDGQYYFNDDGIMQVGFVTINDKVFYFSDSGIIESGVQNI
DDNYFYIDDNGIVQIGVFDTSDGYKYFAPANTVNDNIYGQAVEYSGLVRV
GEDVYYFGETYTIETGWIYDMENESDKYYFNPETKKACKGINLIDDIKYY
FDEKGIMRTGLISFENNNYYFNENGEMQFGYINIEDKMFYFGEDGVMQIG
VFNTPDGFKYFAHQNTLDENFEGESINYTGWLDLDEKRYYFTDEYIAATG
SVIIDGEEYYFDPDTAQLVISE

SEQ ID NO: 13, STF2R3′(2xRBD-A). (Double full length RBDs connected by flexible linkers replaced D3 of flagellin. TcdA, full receptor binding domain, residues 1812-2710 of C. diff. antigen in italics. Note: Single underline, short repeat; double underline, long repeat)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGFKI
IDNKTYYYDEDSKLVKGLININNSLFYDPIEFNLVTGWQTINGKKYYFD
INTGAALTSYKIINGKHFYFNNDGVMQLGVFKGPDGFEYFAPANTQNNNI
EGQAIVYQSKFLTLNGKKYYFDNNSKAVTGWRIINNEKYYFNPNNAIAAV
GLQVIDNNKYYFNPDTAIISKGWQTVNGSRYYFDTDTAIAFNGYKTIDGK
HFYFDSDCVVKIGVFSTSNGFEYFAPANTYNNNIEGQAIVYQSKFLTLNG
KKYYFDNNSKAVTGWQTIDSKKYYFNTNTAEAATGWQTIDGKKYYFNTNT
AEAATGWQTIDGKKYYFNTNTAIASTGYTIINGKHFYFNTDGIMQIGVFK
GPNGFEYFAPANTDANNIEGQAILYQNEFLTLNGKKYYFGSDSKAVTGWR
IINNKKYYFNPNNAIAAIHLCTINNDKYYFSYDGILQNGYITIERNNFYF
DANNESKMVTGVFKGPNGFEYFAPANTHNNNIEGQAIVYQNKFLTLNGKK
YYFDNDSKAVTGWQTIDGKKYYFNLNTAEAATGWQTIDGKKYYFNLNTAE
AATGWQTIDGKKYYFNTNTFIASTGYTSINGKHFYFNTDGIMQIGVFKGP
NGFEYFAPANTDANNIEGQAILYQNKFLTLNGKKYYFGSDSKAVTGLRTI
DGKKYYFNTNTAVAVTGWQTINGKKYYFNTNTSIASTGYTIISGKHFYFN
TDGIMQIGVFKGPDGFEYFAPANTDANNIEGQAIRYQNRFLYLHDNIYYF
GNNSKAATGWVTIDGNRYYFEPNTAMGANGYKTIDNKNFYFRNGLPQIGV
FKGSNGFEYFAPANTDANNIEGQAIRYQNRFLHLLGKIYYFGNNSKAVTG
WQTINGKVYYFMPDTAMAAAGGLFEIDGVIYFFGVDGVKAPGIYGGGGGF
KIIDNKTYYYDEDSKLVKGLININNSLFYFDPIEFNLVTGWQTINGKKYY
FDINTGAALTSYKIINGKHFYFNNDGVMQLGVFKGPDGFEYFAPANTQNN
NIEGQAIVYQSKFLTLNGKKYYFDNNSKAVTGWRIINNEKYYFNPNNAIA
AVGLQVIDNNKYYFNPDTAIISKGWQTVNGSRYYFDTDTAIAFNGYKTID
GKHFYFDSDCVVKIGVFSTSNGFEYFAPANTYNNNIEGQAIVYQSKFLTL
NGKKYYFDNNSKAVTGWQTIDSKKYYFNTNTAEAATGWQTIDGKKYYFNT
NTAEAATGWQTIDGKKYYFNTNTAIASTGYTIINGKHFYFNTDGIMQIGV
FKGPNGFEYFAPANTDANNIEGQAILYQNEFLTLNGKKYYFGSDSKAVTG
WRIINNKKYYFNPNNAIAAIHLCTINNDKYYFSYDGILQNGYITIERNNF
YFDANNESKMVTGVFKGPNGFEYFAPANTHNNNIEGQAIVYQNKFLTLNG
KKYYFDNDSKAVTGWQTIDGKKYYFNLNTAEAATGWQTIDGKKYYFNLNT
AEAATGWQTIDGKKYYFNTNTFIASTGYTSINGKHFYFNTDGIMQIGVFK
GPNGFEYFAPANTDANNIEGQAILYQNKFLTLNGKKYYFGSDSKAVTGLR
TIDGKKYYFNTNTAVAVTGWQTINGKKYYFNTNTSIASTGYTIISGKHFY
FNTDGIMQIGVFKGPDGFEYFAPANTDANNIEGQAIRYQNRFLYLHDNIY
YFGNNSKAATGWVTIDGNRYYFEPNTAMGANGYKTIDNKNFYFRNGLPQI
GVFKGSNGFEYFAPANTDANNIEGQAIRYQNRFLHLLGKIYYFGNNSKAV
TGWQTINGKVYYFMPDTAMAAAGGLFEIDGVIYFFGVDGVKAPGIYGGGG
ELKDTPAVVSADAKNALIAGGVDATDANGAELVKMSYTDKNGKTIEGGYA
LKAGDKYYAADYDEATGAIKAKTTSYTAADGTTKTAANQLGGVDGKTEVV
TIDGKTYNASKAAGHDFKAQPELAEAAAKTTENPLQKIDAALAQVDALRS
DLGAVQNRFNSAITNLGNTVNNLSEARSRIEDSDYATEVSNMSRAQILQQ
AGTSVLAQANQVPQNVLSLLA

SEQ ID NO: 14, STF2R3′(2xRBD-B). (Double full length RBDs connected by flexible linkers replaced D3 of flagellin. TcdB, full receptor binding double repeat of residues 1814-2366 in italics. Note: Single underline, short repeat; double underline, long repeat)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGLVSL
YNEKFYINNFGMMVSGLIYINDSLYYFKPPVNNLITGFVTVGDDKYYFNP
INGGAASIGETIIDDKNYYFNQSGVLQTGVFSTEDGFKYFAPANTLDENL
EGEAIDFTGKLIIDENIYYFDDNYRGAVEWKELDGEMHYFSPETGKAFKG
LNQIGDYKYYFNSDGVMQKGFVSINDNKHYFDDSGVMKVGYTEIDGKHFY
FAENGEMQIGVFNTEDGFKYFAHHNEDLGNEEGEEISYSGILNFNNKIYY
FDDSFTAVVGWKDLEDGSKYYFDEDTAEAYIGLSLINDGQYYFNDDGIMQ
VGFVTINDKVFYFSDSGIIESGVQNIDDNYFYIDDNGIVQIGVFDTSDGY
KYFAPANTVNDNIYGQAVEYSGLVRVGEDVYYFGETYTIETGWIYDMENE
SDKYYFNPETKKACKGINLIDDIKYYFDEKGIMRTGLISFENNNYYFNEN
GEMQFGYINIEDKMFYFGEDGVMQIGVFNTPDGFKYFAHQNTLDENFEGE
SINYTGWLDLDEKRYYFTDEYIAATGSVIIDGEEYYFDPDTAQLVISEGG
GGGLVSLYNEKFYINNFGMMVSGLIYINDSLYYFKPPVNNLITGFVTVGD
DKYYFNPINGGAASIGETIIDDKNYYFNQSGVLQTGVFSTEDGFKYFAPA
NTLDENLEGEAIDFTGKLIIDENIYYFDDNYRGAVEWKELDGEMHYFSPE
TGKAFKGLNQIGDYKYYFNSDGVMQKGFVSINDNKHYFDDSGVMKVGYTE
IDGKHFYFAENGEMQIGVFNTEDGFKYFAHHNEDLGNEEGEEISYSGILN
FNNKIYYFDDSFTAVVGWKDLEDGSKYYFDEDTAEAYIGLSLINDGQYYF
NDDGIMQVGFVTINDKVFYFSDSGIIESGVQNIDDNYFYIDDNGIVQIGV
FDTSDGYKYFAPANTVNDNIYGQAVEYSGLVRVGEDVYYFGETYTIETGW
IYDMENESDKYYFNPETKKACKGINLIDDIKYYFDEKGIMRTGLISFENN
NYYFNENGEMQFGYINIEDKMFYFGEDGVMQIGVFNTPDGFKYFAHQNTL
DENFEGESINYTGWLDLDEKRYYFTDEYIAATGSVIIDGEEYYFDPDTAQ
LVISEGGGELKDTPAVVSADAKNALIAGGVDATDANGAELVKMSYTDKNG
KTIEGGYALKAGDKYYAADYDEATGAIKAKTTSYTAADGTTKTAANQLGG
VDGKTEVVTIDGKTYNASKAAGHDFKAQPELAEAAAKTTENPLQKIDAAL
AQVDALRSDLGAVQNRFNSAITNLGNTVNNLSEARSRIEDSDYATEVSNM
SRAQILQQAGTSVLAQANQVPQNVLSLLA

SEQ ID NO: 17, STF2.GTD-A2 (C-terminal fusion with TcdA GTD, D269A+R272A+Y283A+D285A+D287A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLASLISKEELIKLAYSIRPRENEYKTILTNLDEYNKLTTNNNENKY
LQLKKLNESIDVFMNKYKTSSRNRALSNLKKDILKEVILIKNSNTSPVEK
NLHFVWIGGEVSDIALEYIKQWADINAEYNIKLWYDSEAFLVNTLKKAIV
ESSTTEALQLLEEEIQNPQFDNMKFYKKRMEFIYDRQKRFINYYKSQINK
PTVPTIDDIIKSHLVSEYNRDETVLESYRTNSLRKINSNHGIDIRANSLF
TEQELLNIYSQELLNRGNLAAASAIVALLALKNFGGVALAVAMLPGIHSD
LFKTISRPSSIGLDRWEMIKLEAIMKYKKYINNYTSENFDKLDQQLKDNF
KLIIESKSEKSEIFSKLENLNVSDLEIKIAFALGSVINQALISKQGSYLT
NLVIEQVKNRYQFLNQHLNPAIESDNNFTDTTKIFHDSLFNSATAENSMF
LTKIAPYLQVGFMPEARSTISLSGPGAYASAYYDFINLQENTIEKTLKAS
DLIEFKFPENNLSQLTEQEINSLWSFDQASAKYQFEKYVRDYTGGSLSED

SEQ ID NO: 18, STF2.GTD-B2 (C-terminal fusion with TcdB GTD, D270A+R273A+Y284A+D286A+D288A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLASLVNRKQLEKMANVRFRTQEDEYVAILDALEEYHNMSENTVVEK
YLKLKDINSLTDIYIDTYKKSGRNKALKKFKEYLVTEVLELKNNNLTPVE
KNLHFVWIGGQINDTAINYINQWKDVNSDYNVNVFYDSNAFLINTLKKTV
VESAINDTLESFRENLNDPRFDYNKFFRKRMEIIYDKQKNFINYYKAQRE
ENPELIIDDIVKTYLSNEYSKEIDELNTYIEESLNKITQNSGNDVRNFEE
FKNGESFNLYEQELVERWNLAAASAILAISALKEIGGMALAVAMLPGIQP
DLFESIEKPSSVTVDFWEMTKLEAIMKYKEYIPEYTSEHFDMLDEEVQSS
FESVLASKSDKSEIFSSLGDMEASPLEVKIAFNSKGIINQGLISVKDSYC
SNLIVKQIENRYKILNNSLNPAISEDNDFNTTTNTFIDSIMAEANADNGR
FMMELGKYLRVGFFPDVKTTINLSGPEAYAAAYQDLLMFKEGSMNIHLIE
ADLRNFEISKTNISQSTEQEMASLWSFDDARAKAQFEEYKRNYFEGSLGE
D

SEQ ID NO: 19, STF2R3′(f1).GTD-A2 (The f1 fragment of TcdA RBD replaced D3 of flagellin with single underlined flexible linkers and C-terminal fusion with TcdA GTD, D269A+R272A+Y283A+D285A+D287A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLASLISKEELIKLAYSIRPRENEYKTILTNLDEYNKLTTNNNENKYLQL
KKLNESIDVFMNKYKTSSRNRALSNLKKDILKEVILIKNSNTSPVEKNLH
FVWIGGEVSDIALEYIKQWADINAEYNIKLWYDSEAFLVNTLKKAIVESS
TTEALQLLEEEIQNPQFDNMKFYKKRMEFIYDRQKRFINYYKSQINKPTV
PTIDDIIKSHLVSEYNRDETVLESYRTNSLRKINSNHGIDIRANSLFTEQ
ELLNIYSQELLNRGNLAAAS IV LLALKNFGGVALAVAMLPGIHSDLFKTI
DLFKDLFKTISRPSSIGLDRWEMIKLEAIMKYKKYINNYTSENFDKLDQQ
SKSEKSEIFSKLENLNVSDLEIKIAFALGSVINQALISKQGSYLTNLVIE
QVKNRYQFLNQHLNPAIESDNNFTDTTKIFHDSLFNSATAENSMFLTKIA
PYLQVGFMPEARSTISLSGPGAYASAYYDFINLQENTIEKTLKASDLIEF
KFPENNLSQLTEQEINSLWSFDQASAKYQFEKYVRDYTGGSLSED

SEQ ID NO: 20, STF2R3′(f1).GTD-B2 (The f1 fragment of TcdB RBD replaced D3 of flagellin with single underlined flexible linkers and C-terminal fusion with TcdB GTD, D270A+R273A+Y284A+D286A+D288A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGKY
YFNPETKKACKGINLIDDIKYYFDEKGIMRTGLISFENNNYYFNENGEMQ
FGYINIEDKMFYFGEDGVMQIGVFNTPDGFKYFAHQNTLDENFEGESINY
TGWLDLDEKRYYFTDEYIAATGSVIIDGEEYYFDPDTAQLVISEGGGGEL
KDTPAVVSADAKNALIAGGVDATDANGAELVKMSYTDKNGKTIEGGYALK
AGDKYYAADYDEATGAIKAKTTSYTAADGTTKTAANQLGGVDGKTEVVTI
DGKTYNASKAAGHDFKAQPELAEAAAKTTENPLQKIDAALAQVDALRSDL
GAVQNRFNSAITNLGNTVNNLSEARSRIEDSDYATEVSNMSRAQILQQAG
TSVLAQANQVPQNVLSLLAASLVNRKQLEKMANVRFRTQEDEYVAILDALE
EYHNMSENTVVEKYLKLKDINSLTDIYIDTYKKSGRNKALKKFKEYLVTE
VLELKNNNLTPVEKNLHFVWIGGQINDTAINYINQWKDVNSDYNVNVFYD
SNAFLINTLKKTVVESAINDTLESFRENLNDPRFDYNKFFRKRMEIIYDK
QKNFINYYKAQREENPELIIDDIVKTYLSNEYSKEIDELNTYIEESLNKI
TQNSGNDVRNFEEFKNGESFNLYEQELVERWNLAAASAILAISALKEIGG
MALAVAMLPGIQPDLFESIEKPSSVTVDFWEMTKLEAIMKYKEYIPEYTS
EHFDMLDEEVQSSFESVLASKSDKSEIFSSLGDMEASPLEVKIAFNSKGI
INQGLISVKDSYCSNLIVKQIENRYKILNNSLNPAISEDNDFNTTTNTFI
DSIMAEANADNGRFMMELGKYLRVGFFPDVKTTINLSGPEAYAAAYQDLL
MFKEGSMNIHLIEADLRNFEISKTNISQSTEQEMASLWSFDDARAKAQFE
EYKRNYFEGSLGED

SEQ ID NO: 21, STF2.CPD-A2 (C-terminal fusion with TcdA Cysteine Protease Domain, D589A+H655A+C700A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLASEDNGVDFNKNTALDKNYLLNNKIPSNNVEEAGSKNYVHYIIQL
QGADISYEATCNLFSKNPKNSIIIQRNMNESAKSYFLSDDGESILELNKY
RIPERLKNKEKVKVTFIGAGKDEFNTSEFARLSVDSLSNEISSFLDTIKL
DISPKNVEVNLLGANMFSYDFNVEETYPGKLLLSIMDKITSTLPDVNKNS
ITIGANQYEVRINSEGRKELLAHSGKWINKEEAIMSDLSSKEYIFFDSID
NKLKAKSKNIPGLASISEDIKT

SEQ ID NO: 22, STF2.CPD-B2 (C-terminal fusion with TcdB Cysteine Protease Domain, D587A+H653A+C698A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLAGEDDNLDFSQNIVVDKEYLLEKISSLARSSERGYIHYIVQLQGA
KISYEAACNLFAKTPYDSVLFQKNIEDSEIAYYYNPGDGEIQEIDKYKIP
SIISDRPKIKLTFIGAGKDEFNTDIFAGFDVDSLSTEIEAAIDLAKEDIS
PKSIEINLLGANMFSYSINVEETYPGKLLLKVKDKISELMPSISQDSIIV
SANQYEVRINSEGRRELLDHSGEWINKEESIIKDISSKEYISFNPKENKI
TVKSKNLPELSTLLQEIRN

SEQ ID NO: 23, STF2R3′(f1).CPD-A2 (The f1 fragment of TcdA RBD replaced D3 of flagellin with single underlined flexible linkers and C-terminal fusion with TcdA CPD, D589A+H655A+C700A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLASEDNGVDFNKNTALDKNYLLNNKIPSNNVEEAGSKNYVHYIIQLQGA
DISYEATCNLFSKNPKNSIIIQRNMNESAKSYFLSDDGESILELNKYRIP
ERLKNKEKVKVTFIGAGKDEFNTSEFARLSVDSLSNEISSFLDTIKLDIS
PKNVEVNLLGANMFSYDFNVEETYPGKLLLSIMDKITSTLPDVNKNSITI
GANQYEVRINSEGRKELLAHSGKWINKEEAIMSDLSSKEYIFFDSIDNKL
KAKSKNIPGLASISEDIKT

SEQ ID NO: 24, STF2R3(f1).CPD-B2 (The f1 fragment of TcdB RBD replaced D3 of flagellin with single underlined flexible linkers and C-terminal fusion with TcdB CPD, D587A+H653A+C698A to abolish enzymatic activity; C. diff. antigen is double underlined).

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGKY
YFNPETKKACKGINLIDDIKYYFDEKGIMRTGLISFENNNYYFNENGEMQ
FGYINIEDKMFYFGEDGVMQIGVFNTPDGFKYFAHQNTLDENFEGESINY
TGWLDLDEKRYYFTDEYIAATGSVIIDGEEYYFDPDTAQLVISEGGGGEL
KDTPAVVSADAKNALIAGGVDATDANGAELVKMSYTDKNGKTIEGGYALK
AGDKYYAADYDEATGAIKAKTTSYTAADGTTKTAANQLGGVDGKTEVVTI
DGKTYNASKAAGHDFKAQPELAEAAAKTTENPLQKIDAALAQVDALRSDL
GAVQNRFNSAITNLGNTVNNLSEARSRIEDSDYATEVSNMSRAQILQQAG
TSVLAQANQVPQNVLSLLAGEDDNLDFSQNIVVDKEYLLEKISSLARSSE
RGYIHYIVQLQGAKISYEAACNLFAKTPYDSVLFQKNIEDSEIAYYYNPG
DGEIQEIDKYKIPSIISDRPKIKLTFIGAGKDEFNTDIFAGFDVDSLSTE
IEAAIDLAKEDISPKSIEINLLGANMFSYSINVEETYPGKLLLKVKDKIS
ELMPSISQDSIIVSANQYEVRINSEGRRELLDHSGEWINKEESIIKDISS
KEYISFNPKENKITVKSKNLPELSTLLQEIRN

SEQ ID NO: 25, HL928, STF2.RBD-A2 (C-terminal fusion with full receptor binding domain with residuesl8l2-2710 of TcdA; 40 repeats of C. diff. antigen in italics. Note: Single underline, short repeat; double underline, long repeat).

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLAGFKIIDNKTYYYDEDSKLVKGLININNSLFYFDPIEFNLVTGWQ
TINGKKYYFDINTGAALTSYKIINGKHFYFNNDGVMQLGVFKGPDGFEYF
APANTQNNNIEGQAIVYQSKFLTLNGKKYYFDNNSKAVTGWRIINNEKYY
FNPNNAIAAVGLQVIDNNKYYFNPDTAIISKGWQTVNGSRYYFDTDTAIA
FNGYKTIDGKHFYFDSDCVVKIGVFSTSNGFEYFAPANTYNNNIEGQAIV
YQSKFLTLNGKKYYFDNNSKAVTGWQTIDSKKYYFNTNTAEAATGWQTID
GKKYYFNTNTAEAATGWQTIDGKKYYFNTNTAIASTGYTIINGKHFYFNT
DGIMQIGVFKGPNGFEYFAPANTDANNIEGQAILYQNEFLTLNGKKYYFG
SDSKAVTGWRIINNKKYYFNPNNAIAAIHLCTINNDKYYFSYDGILQNGY
ITIERNNFYFDANNESKMVTGVFKGPNGFEYFAPANTHNNNIEGQAIVYQ
NKFLTLNGKKYYFDNDSKAVTGWQTIDGKKYYFNLNTAEAATGWQTIDGK
KYYFNLNTAEAATGWQTIDGKKYYFNTNTFIASTGYTSINGKHFYFNTDG
IMQIGVFKGPNGFEYFAPANTDANNIEGQAILYQNKFLTLNGKKYYFGSD
SKAVTGLRTIDGKKYYFNTNTAVAVTGWQTINGKKYYFNTNTSIASTGYT
IISGKHFYFNTDGIMQIGVFKGPDGFEYFAPANTDANNIEGQAIRYQNRF
LYLHDNIYYFGNNSKAATGWVTIDGNRYYFEPNTAMGANGYKTIDNKNFY
FRNGLPQIGVFKGSNGFEYFAPANTDANNIEGQAIRYQNRFLHLLGKIYY
FGNNSKAVTGWQTINGKVYYFMPDTAMAAAGGLFEIDGVIYFFGVDGVKA
PGIYG

SEQ ID NO: 26, HL929, STF2.RBD-B2 (C-terminal fusion with TcdB, full receptor binding residues 1814-2366, 24 repeats of C. diff. antigen in italics. Note: Single underline, short repeat; double underline, long repeat).

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLAGLVSLYNEKFYINNFGMMVSGLIYINDSLYYFKPPVNNLITGFV
TVGDDKYYFNPINGGAASIGETIIDDKNYYFNQSGVLQTGVFSTEDGFKY
FAPANTLDENLEGEAIDFTGKLIIDENIYYFDDNYRGAVEWKELDGEMHY
FSPETGKAFKGLNQIGDYKYYFNSDGVMQKGFVSINDNKHYFDDSGVMKV
GYTEIDGKHFYFAENGEMQIGVFNTEDGFKYFAHHNEDLGNEEGEEISYS
GILNFNNKIYYFDDSFTAVVGWKDLEDGSKYYFDEDTAEAYIGLSLINDG
QYYFNDDGIMQVGFVTINDKVFYFSDSGIIESGVQNIDDNYFYIDDNGIV
QIGVFDTSDGYKYFAPANTVNDNIYGQAVEYSGLVRVGEDVYYFGETYTI
ETGWIYDMENESDKYYFNPETKKACKGINLIDDIKYYFDEKGIMRTGLIS
FENNNYYFNENGEMQFGYINIEDKMFYFGEDGVMQIGVFNTPDGFKYFAH
QNTLDENFEGESINYTGWLDLDEKRYYFTDEYIAATGSVIIDGEEYYFDP
DTAQLVISE

SEQ ID NO: 27, STF2Δ.RBD-A2 (Full length RBD of TcdA in italics fused with STF2Δ)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQTTENPLQKIDAALAQVDALRSDLG
AVQNRFNSAITNLGNTVNNLSEARSRIEDSDYATEVSNMSRAQILQQAGT
SVLAQANQVPQNVLSLLAGFKIIDNKTYYYDEDSKLVKGLININNSLFYF
DPIEFNLVTGWQTINGKKYYFDINTGAALTSYKIINGKHFYFNNDGVMQL
GVFKGPDGFEYFAPANTQNNNIEGQAIVYQSKFLTLNGKKYYFDNNSKAV
TGWRIINNEKYYFNPNNAIAAVGLQVIDNNKYYFNPDTAIISKGWQTVNG
SRYYFDTDTAIAFNGYKTIDGKHFYFDSDCVVKIGVFSTSNGFEYFAPAN
TYNNNIEGQAIVYQSKFLTLNGKKYYFDNNSKAVTGWQTIDSKKYYFNTN
TAEAATGWQTIDGKKYYFNTNTAEAATGWQTIDGKKYYFNTNTAIASTGY
TIINGKHFYFNTDGIMQIGVFKGPNGFEYFAPANTDANNIEGQAILYQNE
FLTLNGKKYYFGSDSKAVTGWRIINNKKYYFNPNNAIAAIHLCTINNDKY
YFSYDGILQNGYITIERNNFYFDANNESKMVTGVFKGPNGFEYFAPANTH
NNNIEGQAIVYQNKFLTLNGKKYYFDNDSKAVTGWQTIDGKKYYFNLNTA
EAATGWQTIDGKKYYFNLNTAEAATGWQTIDGKKYYFNTNTFIASTGYTS
INGKHFYFNTDGIMQIGVFKGPNGFEYFAPANTDANNIEGQAILYQNKFL
TLNGKKYYFGSDSKAVTGLRTIDGKKYYFNTNTAVAVTGWQTINGKKYYF
NTNTSIASTGYTIISGKHFYFNTDGIMQIGVFKGPDGFEYFAPANTDANN
IEGQAIRYQNRFLYLHDNIYYFGNNSKAATGWVTIDGNRYYFEPNTAMGA
NGYKTIDNKNFYFRNGLPQIGVFKGSNGFEYFAPANTDANNIEGQAIRYQ
NRFLHLLGKIYYFGNNSKAVTGWQTINGKVYYFMPDTAMAAAGGLFEIDG
VIYFFGVDGVKAPGIYG

SEQ ID NO: 28, STF2Δ.RBD-B2 (Full length RBD of TcdB in italics fused with STF2Δ)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQTTENPLQKIDAALAQVDALRSDLG
AVQNRFNSAITNLGNTVNNLSEARSRIEDSDYATEVSNMSRAQILQQAGT
SVLAQANQVPQNVLSLLAGLVSLYNEKFYINNFGMMVSGLIYINDSLYYF
KPPVNNLITGFVTVGDDKYYFNPINGGAASIGETIIDDKNYYFNQSGVLQ
TGVFSTEDGFKYFAPANTLDENLEGEAIDFTGKLIIDENIYYFDDNYRGA
VEWKELDGEMHYFSPETGKAFKGLNQIGDYKYYFNSDGVMQKGFVSINDN
KHYFDDSGVMKVGYTEIDGKHFYFAENGEMQIGVFNTEDGFKYFAHHNED
LGNEEGEEISYSGILNFNNKIYYFDDSFTAVVGWKDLEDGSKYYFDEDTA
EAYIGLSLINDGQYYFNDDGIMQVGFVTINDKVFYFSDSGIIESGVQNID
DNYFYIDDNGIVQIGVFDTSDGYKYFAPANTVNDNIYGQAVEYSGLVRVG
EDVYYFGETYTIETGWIYDMENESDKYYFNPETKKACKGINLIDDIKYYF
DEKGIMRTGLISFENNNYYFNENGEMQFGYINIEDKMFYFGEDGVMQIGV
FNTPDGFKYFAHQNTLDENFEGESINYTGWLDLDEKRYYFTDEYIAATGS
VIIDGEEYYFDPDTAQLVISE

Mixing RBD-A and B in one construct

SEQ ID NO: 29, STF2R3′(f1-A).C.RBD-B2 (The f1 fragments of TcdA is used to replace D3 of flagellin with single underlined flexible linkers and C. diff. antigen is double underlined; the full RBD-B C. diff. antigen in italics is fused to the C-terminal of flagellin; Note: Single underline, short repeat; double underline, long repeat)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLAGLVSLYNEKFYINNFGMMVSGLIYINDSLYYFKPPVNNLITGFVTVG
DDKYYFNPINGGAASIGETIIDDKNYYFNQSGVLQTGVFSTEDGFKYFAP
ANTLDENLEGEAIDFTGKLIIDENIYYFDDNYRGAVEWKELDGEMHYFSP
ETGKAFKGLNQIGDYKYYFNSDGVMQKGFVSINDNKHYFDDSGVMKVGYT
EIDGKHFYFAENGEMQIGVFNTEDGFKYFAHHNEDLGNEEGEEISYSGIL
NFNNKIYYFDDSFTAVVGWKDLEDGSKYYFDEDTAEAYIGLSLINDGQYY
FNDDGIMQVGFVTINDKVFYFSDSGIIESGVQNIDDNYFYIDDNGIVQIG
VFDTSDGYKYFAPANTVNDNIYGQAVEYSGLVRVGEDVYYFGETYTIETG
WIYDMENESDKYYFNPETKKACKGINLIDDIKYYFDEKGIMRTGLISFEN
NNYYFNENGEMQFGYINIEDKMFYFGEDGVMQIGVFNTPDGFKYFAHQNT
LDENFEGESINYTGWLDLDEKRYYFTDEYIAATGSVIIDGEEYYFDPDTA
QLVISE

SEQ ID NO: 30, STF2R3′(f1-B).C.RBD-A2 (The f1 fragments of TcdB is used to replace D3 of flagellin with single underlined flexible linkers and C. diff. antigen is double underlined; the full RBD-A C. diff. antigen in italics is fused to the C-terminal of flagellin; Note: Single underline, short repeat; double underline, long repeat)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGKY
YFNPETKKACKGINLIDDIKYYFDEKGIMRTGLISFENNNYYFNENGEMQ
FGYINIEDKMFYFGEDGVMQIGVFNTPDGFKYFAHQNTLDENFEGESINY
TGWLDLDEKRYYFTDEYIAATGSVIIDGEEYYFDPDTAQLVISEGGGGEL
KDTPAVVSADAKNALIAGGVDATDANGAELVKMSYTDKNGKTIEGGYALK
AGDKYYAADYDEATGAIKAKTTSYTAADGTTKTAANQLGGVDGKTEVVTI
DGKTYNASKAAGHDFKAQPELAEAAAKTTENPLQKIDAALAQVDALRSDL
GAVQNRFNSAITNLGNTVNNLSEARSRIEDSDYATEVSNMSRAQILQQAG
TSVLAQANQVPQNVLSLLAGFKIIDNKTYYYDEDSKLVKGLININNSLFY
FDPIEFNLVTGWQTINGKKYYFDINTGAALTSYKIINGKHFYFNNDGVMQ
LGVFKGPDGFEYFAPANTQNNNIEGQAIVYQSKFLTLNGKKYYFDNNSKA
VTGWRIINNEKYYFNPNNAIAAVGLQVIDNNKYYFNPDTAIISKGWQTVN
GSRYYFDTDTAIAFNGYKTIDGKHFYFDSDCVVKIGVFSTSNGFEYFAPA
NTYNNNIEGQAIVYQSKFLTLNGKKYYFDNNSKAVTGWQTIDSKKYYFNT
NTAEAATGWQTIDGKKYYFNTNTAEAATGWQTIDGKKYYFNTNTAIASTG
YTIINGKHFYFNTDGIMQIGVFKGPNGFEYFAPANTDANNIEGQAILYQN
EFLTLNGKKYYFGSDSKAVTGWRIINNKKYYFNPNNAIAAIHLCTINNDK
YYFSYDGILQNGYITIERNNFYFDANNESKMVTGVFKGPNGFEYFAPANT
HNNNIEGQAIVYQNKFLTLNGKKYYFDNDSKAVTGWQTIDGKKYYFNLNT
AEAATGWQTIDGKKYYFNLNTAEAATGWQTIDGKKYYFNTNTFIASTGYT
SINGKHFYFNTDGIMQIGVFKGPNGFEYFAPANTDANNIEGQAILYQNKF
LTLNGKKYYFGSDSKAVTGLRTIDGKKYYFNTNTAVAVTGWQTINGKKYY
FNTNTSIASTGYTIISGKHFYFNTDGIMQIGVFKGPDGFEYFAPANTDAN
NIEGQAIRYQNRFLYLHDNIYYFGNNSKAATGWVTIDGNRYYFEPNTAMG
ANGYKTIDNKNFYFRNGLPQIGVFKGSNGFEYFAPANTDANNIEGQAIRY
QNRFLHLLGKIYYFGNNSKAVTGWQTINGKVYYFMPDTAMAAAGGLFEID
GVIYFFGVDGVKAPGIYG

SEQ ID NO: 31 STF2R3′(f1).RBD (The f1 fragments of TcdA is used to replace D3 of flagellin with single underlined flexible linkers and C. diff. antigen is double underlined; the TcdA-partial repeats in italics that resembles TcdB in pattern)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANGGGGGNR
YYFEPNTAMGANGYKTIDNKNFYFRNGLPQIGVFKGSNGFEYFAPANTDA
NNIEGQAIRYQNRFLHLLGKIYYFGNNSKAVTGWQTINGKVYYFMPDTAM
AAAGGLFEIDGVIYFFGVDGVKAPGIYGGGGGELKDTPAVVSADAKNALI
AGGVDATDANGAELVKMSYTDKNGKTIEGGYALKAGDKYYAADYDEATGA
IKAKTTSYTAADGTTKTAANQLGGVDGKTEVVTIDGKTYNASKAAGHDFK
AQPELAEAAAKTTENPLQKIDAALAQVDALRSDLGAVQNRFNSAITNLGN
TVNNLSEARSRIEDSDYATEVSNMSRAQILQQAGTSVLAQANQVPQNVLS
LLAISKGWQTVNGSRYYFDTDTAIAFNGYKTIDGKHFYFDSDCVVKIGVF
STSNGFEYFAPANTYNNNIEGQAIVYQSKFLTLNGKKYYFDNNSKAVTGW
QTIDSKKYYFNTNTAEAATGWQTIDGKKYYFNTNTAEAATGWQTIDGKKY
YFNTNTAIASTGYTIINGKHFYFNTDGIMQIGVFKGPNGFEYFAPANTDA
NNIEGQAILYQNEFLTLNGKKYYFGSDSKAVTGWRIINNKKYYFNPNNAI
AAIHLCTINNDKYYFSYDGILQNGYITIERNNFYFDANNESKMVTGVFKG
PNGFEYFAPANTHNNNIEGQAIVYQNKFLTLNGKKYYFDNDSKAVTGWQT
IDGKKYYFNLNTAEAATGWQTIDGKKYYFNLNTAEAAT

SEQ ID NO: 32, STF2.GTD-A1 (flagellin I423A C-terminal fusion with TcdA N-terminal Glucosyltransferase Domain (GTD), D269N+D285N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLASLISKEELIKLAYSIRPRENEYKTILTNLDEYNKLTTNNNENKY
LQLKKLNESIDVFMNKYKTSSRNRALSNLKKDILKEVILIKNSNTSPVEK
NLHFVWIGGEVSDIALEYIKQWADINAEYNIKLWYDSEAFLVNTLKKAIV
ESSTTEALQLLEEEIQNPQFDNMKFYKKRMEFIYDRQKRFINYYKSQINK
PTVPTIDDIIKSHLVSEYNRDETVLESYRTNSLRKINSNHGIDIRANSLF
TEQELLNIYSQELLNRGNLAAASNIVRLLALKNFGGVYLNVDMLPGIHSD
LFKTISRPSSIGLDRWEMIKLEAIMKYKKYINNYTSENFDKLDQQLKDNF
KLIIESKSEKSEIFSKLENLNVSDLEIKIAFALGSVINQALISKQGSYLT
NLVIEQVKNRYQFLNQHLNPAIESDNNFTDTTKIFHDSLFNSATAENSMF
LTKIAPYLQVGFMPEARSTISLSGPGAYASAYYDFINLQENTIEKTLKAS
DLIEFKFPENNLSQLTEQEINSLWSFDQASAKYQFEKYVRDYTGGSLSED

SEQ ID NO: 33, STF2.GTD-A1 (D3 deleted flagellin I423A and C-terminal fusion with TcdA GTD, D269N+D285N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLASLISKEELIKLAYSIRPRENEYKTILTNLDEYNKLTTNNNENKYL
QLKKLNESIDVFMNKYKTSSRNRALSNLKKDILKEVILIKNSNTSPVEKN
LHFVWIGGEVSDIALEYIKQWADINAEYNIKLWYDSEAFLVNTLKKAIVE
SSTTEALQLLEEEIQNPQFDNMKFYKKRMEFIYDRQKRFINYYKSQINKP
TVPTIDDIIKSHLVSEYNRDETVLESYRTNSLRKINSNHGIDIRANSLFT
EQELLNIYSQELLNRGNLAAASNIVRLLALKNFGGVYLNVDMLPGIHSDL
FKTISRPSSIGLDRWEMIKLEAIMKYKKYINNYTSENFDKLDQQLKDNFK
LIIESKSEKSEIFSKLENLNVSDLEIKIAFALGSVINQALISKQGSYLTN
LVIEQVKNRYQFLNQHLNPAIESDNNFTDTTKIFHDSLFNSATAENSMFL
TKIAPYLQVGFMPEARSTISLSGPGAYASAYYDFINLQENTIEKTLKASD
LIEFKFPENNLSQLTEQEINSLWSFDQASAKYQFEKYVRDYTGGSLSED

SEQ ID NO: 34, STF2.GTD-B1 (flagellin I423A C-terminal fusion with TcdB GTD, D270N+D286N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLASLVNRKQLEKMANVRFRTQEDEYVAILDALEEYHNMSENTVVEK
YLKLKDINSLTDIYIDTYKKSGRNKALKKFKEYLVTEVLELKNNNLTPVE
KNLHFVWIGGQINDTAINYINQWKDVNSDYNVNVFYDSNAFLINTLKKTV
VESAINDTLESFRENLNDPRFDYNKFFRKRMEIIYDKQKNFINYYKAQRE
ENPELIIDDIVKTYLSNEYSKEIDELNTYIEESLNKITQNSGNDVRNFEE
FKNGESFNLYEQELVERWNLAAASNILRISALKEIGGMYLNVDMLPGIQP
DLFESIEKPSSVTVDFWEMTKLEAIMKYKEYIPEYTSEHFDMLDEEVQSS
FESVLASKSDKSEIFSSLGDMEASPLEVKIAFNSKGIINQGLISVKDSYC
SNLIVKQIENRYKILNNSLNPAISEDNDFNTTTNTFIDSIMAEANADNGR
FMMELGKYLRVGFFPDVKTTINLSGPEAYAAAYQDLLMFKEGSMNIHLIE
ADLRNFEISKTNISQSTEQEMASLWSFDDARAKAQFEEYKRNYFEGSLGE
D

SEQ ID NO: 35, STF2.GTD-B1 (D3 deleted flagellin I423A and C-terminal fusion with TcdB GTD, D270N+D286N to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLASLVNRKQLEKMANVRFRTQEDEYVAILDALEEYHNMSENTVVEKY
LKLKDINSLTDIYIDTYKKSGRNKALKKFKEYLVTEVLELKNNNLTPVEK
NLHFVWIGGQINDTAINYINQWKDVNSDYNVNVFYDSNAFLINTLKKTVV
ESAINDTLESFRENLNDPRFDYNKFFRKRMEIIYDKQKNFINYYKAQREE
NPELIIDDIVKTYLSNEYSKEIDELNTYIEESLNKITQNSGNDVRNFEEF
KNGESFNLYEQELVERWNLAAASNILRISALKEIGGMYLNVDMLPGIQPD
LFESIEKPSSVTVDFWEMTKLEAIMKYKEYIPEYTSEHFDMLDEEVQSSF
ESVLASKSDKSEIFSSLGDMEASPLEVKIAFNSKGIINQGLISVKDSYCS
NLIVKQIENRYKILNNSLNPAISEDNDFNTTTNTFIDSIMAEANADNGRF
MMELGKYLRVGFFPDVKTTINLSGPEAYAAAYQDLLMFKEGSMNIHLIEA
DLRNFEISKTNISQSTEQEMASLWSFDDARAKAQFEEYKRNYFEGSLGED

SEQ ID NO: 36, STF2.GTD-A2 (flagellin I423A C-terminal fusion with TcdA GTD, D269A+R272A+Y283A+D285A+D287A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLASLISKEELIKLAYSIRPRENEYKTILTNLDEYNKLTTNNNENKY
LQLKKLNESIDVFMNKYKTSSRNRALSNLKKDILKEVILIKNSNTSPVEK
NLHFVWIGGEVSDIALEYIKQWADINAEYNIKLWYDSEAFLVNTLKKAIV
ESSTTEALQLLEEEIQNPQFDNMKFYKKRMEFIYDRQKRFINYYKSQINK
PTVPTIDDIIKSHLVSEYNRDETVLESYRTNSLRKINSNHGIDIRANSLF
TEQELLNIYSQELLNRGNLAAASAIVALLALKNFGGVALAVAMLPGIHSD
LFKTISRPSSIGLDRWEMIKLEAIMKYKKYINNYTSENFDKLDQQLKDNF
KLIIESKSEKSEIFSKLENLNVSDLEIKIAFALGSVINQALISKQGSYLT
NLVIEQVKNRYQFLNQHLNPAIESDNNFTDTTKIFHDSLFNSATAENSMF
LTKIAPYLQVGFMPEARSTISLSGPGAYASAYYDFINLQENTIEKTLKAS
DLIEFKFPENNLSQLTEQEINSLWSFDQASAKYQFEKYVRDYTGGSLSED

SEQ ID NO: 37, STF2.GTD-A2 (D3 deleted flagellin I423A and C-terminal fusion with TcdA GTD, D269A+R272A+Y283A+D285A+D287A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLASLISKEELIKLAYSIRPRENEYKTILTNLDEYNKLTTNNNENKYL
QLKKLNESIDVFMNKYKTSSRNRALSNLKKDILKEVILIKNSNTSPVEKN
LHFVWIGGEVSDIALEYIKQWADINAEYNIKLWYDSEAFLVNTLKKAIVE
SSTTEALQLLEEEIQNPQFDNMKFYKKRMEFIYDRQKRFINYYKSQINKP
TVPTIDDIIKSHLVSEYNRDETVLESYRTNSLRKINSNHGIDIRANSLFT
EQELLNIYSQELLNRGNLAAASAIVALLALKNFGGVALAVAMLPGIHSDL
FKTISRPSSIGLDRWEMIKLEAIMKYKKYINNYTSENFDKLDQQLKDNFK
LIIESKSEKSEIFSKLENLNVSDLEIKIAFALGSVINQALISKQGSYLTN
LVIEQVKNRYQFLNQHLNPAIESDNNFTDTTKIFHDSLFNSATAENSMFL
TKIAPYLQVGFMPEARSTISLSGPGAYASAYYDFINLQENTIEKTLKASD
LIEFKFPENNLSQLTEQEINSLWSFDQASAKYQFEKYVRDYTGGSLSED

SEQ ID NO: 38, STF2.GTD-B2 (flagellin I423A C-terminal fusion with TcdB GTD, D270A+R273A+Y284A+D286A+D288A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLASLVNRKQLEKMANVRFRTQEDEYVAILDALEEYHNMSENTVVEK
YLKLKDINSLTDIYIDTYKKSGRNKALKKFKEYLVTEVLELKNNNLTPVE
KNLHFVWIGGQINDTAINYINQWKDVNSDYNVNVFYDSNAFLINTLKKTV
VESAINDTLESFRENLNDPRFDYNKFFRKRMEIIYDKQKNFINYYKAQRE
ENPELIIDDIVKTYLSNEYSKEIDELNTYIEESLNKITQNSGNDVRNFEE
FKNGESFNLYEQELVERWNLAAASAILAISALKEIGGMALAVAMLPGIQP
DLFESIEKPSSVTVDFWEMTKLEAIMKYKEYIPEYTSEHFDMLDEEVQSS
FESVLASKSDKSEIFSSLGDMEASPLEVKIAFNSKGIINQGLISVKDSYC
SNLIVKQIENRYKILNNSLNPAISEDNDFNTTTNTFIDSIMAEANADNGR
FMMELGKYLRVGFFPDVKTTINLSGPEAYAAAYQDLLMFKEGSMNIHLIE
ADLRNFEISKTNISQSTEQEMASLWSFDDARAKAQFEEYKRNYFEGSLGE
D

SEQ ID NO: 39, STF2.GTD-B2 (D3 deleted flagellin I423A and C-terminal fusion with TcdB GTD, D270A+R273A+Y284A+D286A+D288A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLASLVNRKQLEKMANVRFRTQEDEYVAILDALEEYHNMSENTVVEKY
LKLKDINSLTDIYIDTYKKSGRNKALKKFKEYLVTEVLELKNNNLTPVEK
NLHFVWIGGQINDTAINYINQWKDVNSDYNVNVFYDSNAFLINTLKKTVV
ESAINDTLESFRENLNDPRFDYNKFFRKRMEIIYDKQKNFINYYKAQREE
NPELIIDDIVKTYLSNEYSKEIDELNTYIEESLNKITQNSGNDVRNFEEF
KNGESFNLYEQELVERWNLAAASAILAISALKEIGGMALAVAMLPGIQPD
LFESIEKPSSVTVDFWEMTKLEAIMKYKEYIPEYTSEHFDMLDEEVQSSF
ESVLASKSDKSEIFSSLGDMEASPLEVKIAFNSKGIINQGLISVKDSYCS
NLIVKQIENRYKILNNSLNPAISEDNDFNTTTNTFIDSIMAEANADNGRF
MMELGKYLRVGFFPDVKTTINLSGPEAYAAAYQDLLMFKEGSMNIHLIEA
DLRNFEISKTNISQSTEQEMASLWSFDDARAKAQFEEYKRNYFEGSLGED

SEQ ID NO: 40, STF2.CPD-A1 (flagellin I423A C-terminal fusion with TcdA Cysteine Protease Domain (CPD), D589N+H655A+C700S to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLASEDNGVDFNKNTALDKNYLLNNKIPSNNVEEAGSKNYVHYIIQL
QGNDISYEATCNLFSKNPKNSIIIQRNMNESAKSYFLSDDGESILELNKY
RIPERLKNKEKVKVTFIGAGKDEFNTSEFARLSVDSLSNEISSFLDTIKL
DISPKNVEVNLLGSNMFSYDFNVEETYPGKLLLSIMDKITSTLPDVNKNS
ITIGANQYEVRINSEGRKELLAHSGKWINKEEAIMSDLSSKEYIFFDSID
NKLKAKSKNIPGLASISEDIKT

SEQ ID NO: 41, STF2.CPD-A1 (D3 deleted flagellin I423A and C-terminal fusion with TcdA Cysteine Protease Domain, D589N+H655A+C700S to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLASEDNGVDFNKNTALDKNYLLNNKIPSNNVEEAGSKNYVHYIIQLQ
GNDISYEATCNLFSKNPKNSIIIQRNMNESAKSYFLSDDGESILELNKYR
IPERLKNKEKVKVTFIGAGKDEFNTSEFARLSVDSLSNEISSFLDTIKLD
ISPKNVEVNLLGSNMFSYDFNVEETYPGKLLLSIMDKITSTLPDVNKNSI
TIGANQYEVRINSEGRKELLAHSGKWINKEEAIMSDLSSKEYIFFDSIDN
KLKAKSKNIPGLASISEDIKT

SEQ ID NO: 42, STF2.CPD-B1 (flagellin I423A C-terminal fusion with TcdB Cysteine Protease Domain, D587N+H653A+C698S to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLAGEDDNLDFSQNIVVDKEYLLEKISSLARSSERGYIHYIVQLQGN
KISYEAACNLFAKTPYDSVLFQKNIEDSEIAYYYNPGDGEIQEIDKYKIP
SIISDRPKIKLTFIGAGKDEFNTDIFAGFDVDSLSTEIEAAIDLAKEDIS
PKSIEINLLGSNMFSYSINVEETYPGKLLLKVKDKISELMPSISQDSIIV
SANQYEVRINSEGRRELLDHSGEWINKEESIIKDISSKEYISFNPKENKI
TVKSKNLPELSTLLQEIRN

SEQ ID NO: 43, STF2.CPD-B1 (D3 deleted flagellin I423A and C-terminal fusion with TcdB Cysteine Protease Domain, D587N+H653A+C698S to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLAGEDDNLDFSQNIVVDKEYLLEKISSLARSSERGYIHYIVQLQGNK
ISYEAACNLFAKTPYDSVLFQKNIEDSEIAYYYNPGDGEIQEIDKYKIPS
IISDRPKIKLTFIGAGKDEFNTDIFAGFDVDSLSTEIEAAIDLAKEDISP
KSIEINLLGSNMFSYSINVEETYPGKLLLKVKDKISELMPSISQDSIIVS
ANQYEVRINSEGRRELLDHSGEWINKEESIIKDISSKEYISFNPKENKIT
VKSKNLPELSTLLQEIRN

SEQ ID NO: 44, STF2.CPD-A2 (flagellin I423A C-terminal fusion with TcdA Cysteine Protease Domain, D589A+H655A+C700A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLASEDNGVDFNKNTALDKNYLLNNKIPSNNVEEAGSKNYVHYIIQL
QGADISYEATCNLFSKNPKNSIIIQRNMNESAKSYFLSDDGESILELNKY
RIPERLKNKEKVKVTFIGAGKDEFNTSEFARLSVDSLSNEISSFLDTIKL
DISPKNVEVNLLGANMFSYDFNVEETYPGKLLLSIMDKITSTLPDVNKNS
ITIGANQYEVRINSEGRKELLAHSGKWINKEEAIMSDLSSKEYIFFDSID
NKLKAKSKNIPGLASISEDIKT

SEQ ID NO: 45, STF2.CPD-A2 (D3 deleted flagellin I423A and C-terminal fusion with TcdA Cysteine Protease Domain, D589A+H655A+C700A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLASEDNGVDFNKNTALDKNYLLNNKIPSNNVEEAGSKNYVHYIIQLQ
GADISYEATCNLFSKNPKNSIIIQRNMNESAKSYFLSDDGESILELNKYR
IPERLKNKEKVKVTFIGAGKDEFNTSEFARLSVDSLSNEISSFLDTIKLD
ISPKNVEVNLLGANMFSYDFNVEETYPGKLLLSIMDKITSTLPDVNKNSI
TIGANQYEVRINSEGRKELLAHSGKWINKEEAIMSDLSSKEYIFFDSIDN
KLKAKSKNIPGLASISEDIKT

SEQ ID NO: 46, STF2.CPD-B2 (flagellin I423A C-terminal fusion with TcdB Cysteine Protease Domain, D587A+H653A+C698A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLAGEDDNLDFSQNIVVDKEYLLEKISSLARSSERGYIHYIVQLQGA
KISYEAACNLFAKTPYDSVLFQKNIEDSEIAYYYNPGDGEIQEIDKYKIP
SIISDRPKIKLTFIGAGKDEFNTDIFAGFDVDSLSTEIEAAIDLAKEDIS
PKSIEINLLGANMFSYSINVEETYPGKLLLKVKDKISELMPSISQDSIIV
SANQYEVRINSEGRRELLDHSGEWINKEESIIKDISSKEYISFNPKENKI
TVKSKNLPELSTLLQEIRN

SEQ ID NO: 47, STF2.CPD-B2 (D3 deleted flagellin I423A and C-terminal fusion with TcdB Cysteine Protease Domain, D587A+H653A+C698A to abolish enzymatic activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLAGEDDNLDFSQNIVVDKEYLLEKISSLARSSERGYIHYIVQLQGAK
ISYEAACNLFAKTPYDSVLFQKNIEDSEIAYYYNPGDGEIQEIDKYKIPS
IISDRPKIKLTFIGAGKDEFNTDIFAGFDVDSLSTEIEAAIDLAKEDISP
KSIEINLLGANMFSYSINVEETYPGKLLLKVKDKISELMPSISQDSIIVS
ANQYEVRINSEGRRELLDHSGEWINKEESIIKDISSKEYISFNPKENKIT
VKSKNLPELSTLLQEIRN

SEQ ID NO: 48, STF2.RBD-A2 (flagellin I423A and C-terminal fusion with FULL RECEPTOR BINDING DOMAIN (RBD) 1812-2710 IN ITALICS. 40 REPEATS).

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLAGFKIIDNKTYYYDEDSKLVKGLININNSLFYFDPIEFNLVTGWQ
TINGKKYYFDINTGAALTSYKIINGKHFYFNNDGVMQLGVFKGPDGFEYF
APANTQNNNIEGQAIVYQSKFLTLNGKKYYFDNNSKAVTGWRIINNEKYY
FNPNNAIAAVGLQVIDNNKYYFNPDTAIISKGWQTVNGSRYYFDTDTAIA
FNGYKTIDGKHFYFDSDCVVKIGVFSTSNGFEYFAPANTYNNNIEGQAIV
YQSKFLTLNGKKYYFDNNSKAVTGWQTIDSKKYYFNTNTAEAATGWQTID
GKKYYFNTNTAEAATGWQTIDGKKYYFNTNTAIASTGYTIINGKHFYFNT
DGIMQIGVFKGPNGFEYFAPANTDANNIEGQAILYQNEFLTLNGKKYYFG
SDSKAVTGWRIINNKKYYFNPNNAIAAIHLCTINNDKYYFSYDGILQNGY
ITIERNNFYFDANNESKMVTGVFKGPNGFEYFAPANTHNNNIEGQAIVYQ
NKFLTLNGKKYYFDNDSKAVTGWQTIDGKKYYFNLNTAEAATGWQTIDGK
KYYFNLNTAEAATGWQTIDGKKYYFNTNTFIASTGYTSINGKHFYFNTDG
IMQIGVFKGPNGFEYFAPANTDANNIEGQAILYQNKFLTLNGKKYYFGSD
SKAVTGLRTIDGKKYYFNTNTAVAVTGWQTINGKKYYFNTNTSIASTGYT
IISGKHFYFNTDGIMQIGVFKGPDGFEYFAPANTDANNIEGQAIRYQNRF
LYLHDNIYYFGNNSKAATGWVTIDGNRYYFEPNTAMGANGYKTIDNKNFY
FRNGLPQIGVFKGSNGFEYFAPANTDANNIEGQAIRYQNRFLHLLGKIYY
FGNNSKAVTGWQTINGKVYYFMPDTAMAAAGGLFEIDGVIYFFGVDGVKA
PGIYG

SEQ ID NO: 49, STF2.RBD-A2 (D3 deleted flagellin I423A and C-terminal fusion with FULL RECEPTOR BINDING DOMAIN 1812-2710 IN ITALICS. 40 REPEATS).

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLAGFKIIDNKTYYYDEDSKLVKGLININNSLFYFDPIEFNLVTGWQT
INGKKYYFDINTGAALTSYKIINGKHFYFNNDGVMQLGVFKGPDGFEYFA
PANTQNNNIEGQAIVYQSKFLTLNGKKYYFDNNSKAVTGWRIINNEKYYF
NPNNAIAAVGLQVIDNNKYYFNPDTAIISKGWQTVNGSRYYFDTDTAIAF
NGYKTIDGKHFYFDSDCVVKIGVFSTSNGFEYFAPANTYNNNIEGQAIVY
QSKFLTLNGKKYYFDNNSKAVTGWQTIDSKKYYFNTNTAEAATGWQTIDG
KKYYFNTNTAEAATGWQTIDGKKYYFNTNTAIASTGYTIINGKHFYFNTD
GIMQIGVFKGPNGFEYFAPANTDANNIEGQAILYQNEFLTLNGKKYYFGS
DSKAVTGWRIINNKKYYFNPNNAIAAIHLCTINNDKYYFSYDGILQNGYI
TIERNNFYFDANNESKMVTGVFKGPNGFEYFAPANTHNNNIEGQAIVYQN
KFLTLNGKKYYFDNDSKAVTGWQTIDGKKYYFNLNTAEAATGWQTIDGKK
YYFNLNTAEAATGWQTIDGKKYYFNTNTFIASTGYTSINGKHFYFNTDGI
MQIGVFKGPNGFEYFAPANTDANNIEGQAILYQNKFLTLNGKKYYFGSDS
KAVTGLRTIDGKKYYFNTNTAVAVTGWQTINGKKYYFNTNTSIASTGYTI
ISGKHFYFNTDGIMQIGVFKGPDGFEYFAPANTDANNIEGQAIRYQNRFL
YLHDNIYYFGNNSKAATGWVTIDGNRYYFEPNTAMGANGYKTIDNKNFYF
RNGLPQIGVFKGSNGFEYFAPANTDANNIEGQAIRYQNRFLHLLGKIYYF
GNNSKAVTGWQTINGKVYYFMPDTAMAAAGGLFEIDGVIYFFGVDGVKAP
GIYG

SEQ ID NO: 50, STF2.RBD-B2 (flagellin I423A and C-terminal fusion with FULL RECEPTOR BINDING DOMAIN 1814-2366 in italics, 24 repeats).

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKADAALAQVDALRSDLGAVQNRFNSAITN
LGNTVNNLSEARSRIEDSDYATEVSNMSRAQILQQAGTSVLAQANQVPQN
VLSLLAGLVSLYNEKFYINNFGMMVSGLIYINDSLYYFKPPVNNLITGFV
TVGDDKYYFNPINGGAASIGETIIDDKNYYFNQSGVLQTGVFSTEDGFKY
FAPANTLDENLEGEAIDFTGKLIIDENIYYFDDNYRGAVEWKELDGEMHY
FSPETGKAFKGLNQIGDYKYYFNSDGVMQKGFVSINDNKHYFDDSGVMKV
GYTEIDGKHFYFAENGEMQIGVFNTEDGFKYFAHHNEDLGNEEGEEISYS
GILNFNNKIYYFDDSFTAVVGWKDLEDGSKYYFDEDTAEAYIGLSLINDG
QYYFNDDGIMQVGFVTINDKVFYFSDSGIIESGVQNIDDNYFYIDDNGIV
QIGVFDTSDGYKYFAPANTVNDNIYGQAVEYSGLVRVGEDVYYFGETYTI
ETGWIYDMENESDKYYFNPETKKACKGINLIDDIKYYFDEKGIMRTGLIS
FENNNYYFNENGEMQFGYINIEDKMFYFGEDGVMQIGVFNTPDGFKYFAH
QNTLDENFEGESINYTGWLDLDEKRYYFTDEYIAATGSVIIDGEEYYFDP
DTAQLVISE

SEQ ID NO: 51, STF2.RBD-B2 (D3 deleted flagellin I423A and C-terminal fusion with FULL RECEPTOR BINDING DOMAIN 1814-2366 in italics, 24 repeats).

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAAVVSADAKNA
LIAGGVDATDANGAELVKMSYTDKNGKTIEGGYALKAGDKYYAADYDEAT
GAIKAKTTSYTAADGTTKTAANQLGGVDGKTEVVTIDGKTYNASKAAGHD
FKAQPELAEAAAKTTENPLQKADAALAQVDALRSDLGAVQNRFNSAITNL
GNTVNNLSEARSRIEDSDYATEVSNMSRAQILQQAGTSVLAQANQVPQNV
LSLLAGLVSLYNEKFYINNFGMMVSGLIYINDSLYYFKPPVNNLITGFVT
VGDDKYYFNPINGGAASIGETIIDDKNYYFNQSGVLQTGVFSTEDGFKYF
APANTLDENLEGEAIDFTGKLIIDENIYYFDDNYRGAVEWKELDGEMHYF
SPETGKAFKGLNQIGDYKYYFNSDGVMQKGFVSINDNKHYFDDSGVMKVG
YTEIDGKHFYFAENGEMQIGVFNTEDGFKYFAHHNEDLGNEEGEEISYSG
ILNFNNKIYYFDDSFTAVVGWKDLEDGSKYYFDEDTAEAYIGLSLINDGQ
YYFNDDGIMQVGFVTINDKVFYFSDSGIIESGVQNIDDNYFYIDDNGIVQ
IGVFDTSDGYKYFAPANTVNDNIYGQAVEYSGLVRVGEDVYYFGETYTIE
TGWIYDMENESDKYYFNPETKKACKGINLIDDIKYYFDEKGIMRTGLISF
ENNNYYFNENGEMQFGYINIEDKMFYFGEDGVMQIGVFNTPDGFKYFAHQ
NTLDENFEGESINYTGWLDLDEKRYYFTDEYIAATGSVIIDGEEYYFDPD
TAQLVISE

SEQ ID NO: 52, STF2Δ.RBD-A2 (D2 and D3 deleted flagellin I423A and C-terminal fusion with Full length RBD of TcdA in italics)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQTTENPLQKADAALAQVDALRSDLG
AVQNRFNSAITNLGNTVNNLSEARSRIEDSDYATEVSNMSRAQILQQAGT
SVLAQANQVPQNVLSLLAGFKIIDNKTYYYDEDSKLVKGLININNSLFYF
DPIEFNLVTGWQTINGKKYYFDINTGAALTSYKIINGKHFYFNNDGVMQL
GVFKGPDGFEYFAPANTQNNNIEGQAIVYQSKFLTLNGKKYYFDNNSKAV
TGWRIINNEKYYFNPNNAIAAVGLQVIDNNKYYFNPDTAIISKGWQTVNG
SRYYFDTDTAIAFNGYKTIDGKHFYFDSDCVVKIGVFSTSNGFEYFAPAN
TYNNNIEGQAIVYQSKFLTLNGKKYYFDNNSKAVTGWQTIDSKKYYFNTN
TAEAATGWQTIDGKKYYFNTNTAEAATGWQTIDGKKYYFNTNTAIASTGY
TIINGKHFYFNTDGIMQIGVFKGPNGFEYFAPANTDANNIEGQAILYQNE
FLTLNGKKYYFGSDSKAVTGWRIINNKKYYFNPNNAIAAIHLCTINNDKY
YFSYDGILQNGYITIERNNFYFDANNESKMVTGVFKGPNGFEYFAPANTH
NNNIEGQAIVYQNKFLTLNGKKYYFDNDSKAVTGWQTIDGKKYYFNLNTA
EAATGWQTIDGKKYYFNLNTAEAATGWQTIDGKKYYFNTNTFIASTGYTS
INGKHFYFNTDGIMQIGVFKGPNGFEYFAPANTDANNIEGQAILYQNKFL
TLNGKKYYFGSDSKAVTGLRTIDGKKYYFNTNTAVAVTGWQTINGKKYYF
NTNTSIASTGYTIISGKHFYFNTDGIMQIGVFKGPDGFEYFAPANTDANN
IEGQAIRYQNRFLYLHDNIYYFGNNSKAATGWVTIDGNRYYFEPNTAMGA
NGYKTIDNKNFYFRNGLPQIGVFKGSNGFEYFAPANTDANNIEGQAIRYQ
NRFLHLLGKIYYFGNNSKAVTGWQTINGKVYYFMPDTAMAAAGGLFEIDG
VIYFFGVDGVKAPGIYG

SEQ ID NO: 53, STF2Δ.RBD-B2 (D2 and D3 deleted flagellin I423A and C-terminal fusion with Full length RBD of TcdB in italics)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQTTENPLQKADAALAQVDALRSDLG
AVQNRFNSAITNLGNTVNNLSEARSRIEDSDYATEVSNMSRAQILQQAGT
SVLAQANQVPQNVLSLLAGIVSLYNEKFYINNFGMMVSGLIYINDSLYYF
KPPVNNLITGFVTVGDDKYYFNPINGGAASIGETIIDDKNYYFNQSGVLQ
TGVFSTEDGFKYFAPANTLDENLEGEAIDFTGKLIIDENIYYFDDNYRGA
VEWKELDGEMHYFSPETGKAFKGLNQIGDYKYYFNSDGVMQKGFVSINDN
KHYFDDSGVMKVGYTEIDGKHFYFAENGEMQIGVFNTEDGFKYFAHHNED
LGNEEGEEISYSGILNFNNKIYYFDDSFTAVVGWKDLEDGSKYYFDEDTA
EAYIGLSLINDGQYYFNDDGIMQVGFVTINDKVFYFSDSGIIESGVQNID
DNYFYIDDNGIVQIGVFDTSDGYKYFAPANTVNDNIYGQAVEYSGLVRVG
EDVYYFGETYTIETGWIYDMENESDKYYFNPETKKACKGINLIDDIKYYF
DEKGIMRTGLISFENNNYYFNENGEMQFGYINIEDKMFYFGEDGVMQIGV
FNTPDGFKYFAHQNTLDENFEGESINYTGWLDLDEKRYYFTDEYIAATGS
VIIDGEEYYFDPDTAQLVISE

SEQ ID NO: 57, TcdA N-terminal GTD residues 2-545, D269N+D285N (of full length TcdA, SEQ ID NO: 15) to abolish enzymatic activity

SLISKEELIKLAYSIRPRENEYKTILTNLDEYNKLTTNNNENKYLQLKKL
NESIDVFMNKYKTSSRNRALSNLKKDILKEVILIKNSNTSPVEKNLHFVW
IGGEVSDIALEYIKQWADINAEYNIKLWYDSEAFLVNTLKKAIVESSTTE
ALQLLEEEIQNPQFDNMKFYKKRMEFIYDRQKRFINYYKSQINKPTVPTI
DDIIKSHLVSEYNRDETVLESYRTNSLRKINSNHGIDIRANSLFTEQELL
NIYSQELLNRGNLAAASNIVRLLALKNFGGVYLNVDMLPGIHSDLFKTIS
RPSSIGLDRWEMIKLEAIMKYKKYINNYTSENFDKLDQQLKDNFKLIIES
KSEKSEIFSKLENLNVSDLEIKIAFALGSVINQALISKQGSYLTNLVIEQ
VKNRYQFLNQHLNPAIESDNNFTDTTKIFHDSLFNSATAENSMFLTKIAP
YLQVGFMPEARSTISLSGPGAYASAYYDFINLQENTIEKTLKASDLIEFK
FPENNLSQLTEQEINSLWSFDQASAKYQFEKYVRDYTGGSLSED

SEQ ID NO: 59, TcdA CPD, D589N+H655A+C700S (residues 543-808 of full length TcdA, SEQ ID NO: 15) to abolish enzymatic activity

SEDNGVDFNKNTALDKNYLLNNKIPSNNVEEAGSKNYVHYIIQLQGNDIS
YEATCNLFSKNPKNSIIIQRNMNESAKSYFLSDDGESILELNKYRIPERL
KNKEKVKVTFIGAGKDEFNTSEFARLSVDSLSNEISSFLDTIKLDISPKN
VEVNLLGSNMFSYDFNVEETYPGKLLLSIMDKITSTLPDVNKNSITIGAN
QYEVRINSEGRKELLAHSGKWINKEEAIMSDLSSKEYIFFDSIDNKLKAK
SKNIPGLASISEDIKT

SEQ ID NO: 61, C. difficile, TcdA Receptor Binding Domain or RBD-A1 with 14 repeats residues 2387-2707 (of full length TcdA, SEQ ID NO: 15)

ASTGYTSINGKHFYFNTDGIMQIGVFKGPNGFEYFAPANTDANNIEGQAI
LYQNKFLTLNGKKYYFGSDSKAVTGLRTIDGKKYYFNTNTAVAVTGWQTI
NGKKYYFNTNTSIASTGYTIISGKHFYFNTDGIMQIGVFKGPDGFEYFAP
ANTDANNIEGQAIRYQNRFLYLHDNIYYFGNNSKAATGWVTIDGNRYYFE
PNTAMGANGYKTIDNKNFYFRNGLPQIGVFKGSNGFEYFAPANTDANNIE
GQAIRYQNRFLHLLGKIYYFGNNSKAVTGWQTINGKVYYFMPDTAMAAAG
GLFEIDGVIYFFGVDGVKAPG

SEQ ID NO: 62 Clostridium difficile f1 fragment of TcdA RBD residues 2581-2710 (of full length TcdA, SEQ ID NO: 15) with single underlined flexible linkers

GGGGGNRYYFEPNTAMGANGYKTIDNKNFYFRNGLPQIGVFKGSNGFEYF
APANTDANNIEGQAIRYQNRFLHLLGKIYYFGNNSKAVTGWQTINGKVYY
FMPDTAMAAAGGLFEIDGVIYFFGVDGVKAPGIYGGGGG

SEQ ID NO: 64, TcdB N-terminal GTD residues 2-546, D270N+D286N (of full length TcdB, SEQ ID NO: 16) to abolish enzymatic activity

SLVNRKQLEKMANVRFRTQEDEYVAILDALEEYHNMSENTVVEKYLKLKD
INSLTDIYIDTYKKSGRNKALKKFKEYLVTEVLELKNNNLTPVEKNLHFV
WIGGQINDTAINYINQWKDVNSDYNVNVFYDSNAFLINTLKKTVVESAIN
DTLESFRENLNDPRFDYNKFFRKRMEIIYDKQKNFINYYKAQREENPELI
IDDIVKTYLSNEYSKEIDELNTYIEESLNKITQNSGNDVRNFEEFKNGES
FNLYEQELVERWNLAAASNILRISALKEIGGMYLNVDMLPGIQPDLFESI
EKPSSVTVDFWEMTKLEAIMKYKEYIPEYTSEHFDMLDEEVQSSFESVLA
SKSDKSEIFSSLGDMEASPLEVKIAFNSKGIINQGLISVKDSYCSNLIVK
QIENRYKILNNSLNPAISEDNDFNTTTNTFIDSIMAEANADNGRFMMELG
KYLRVGFFPDVKTTINLSGPEAYAAAYQDLLMFKEGSMNIHLIEADLRNF
EISKTNISQSTEQEMASLWSFDDARAKAQFEEYKRNYFEGSLGED

SEQ ID NO: 66, TcdB CPD residues 544-806, D587N+H653A+C698S (of full length TcdB, SEQ ID NO: 16) to abolish enzymatic activity

GEDDNLDFSQNIVVDKEYLLEKISSLARSSERGYIHYIVQLQGNKISYEA
ACNLFAKTPYDSVLFQKNIEDSEIAYYYNPGDGEIQEIDKYKIPSIISDR
PKIKLTFIGAGKDEFNTDIFAGFDVDSLSTEIEAAIDLAKEDISPKSIEI
NLLGSNMFSYSINVEETYPGKLLLKVKDKISELMPSISQDSIIVSANQYE
VRINSEGRRELLDHSGEWINKEESIIKDISSKEYISFNPKENKITVKSKN
LPELSTLLQEIRN

SEQ ID NO: 68, C. difficile, TcdB Receptor Binding Domain or RBD-B1 with 16 repeats residues 2005-2366 (of full length TcdB, SEQ ID NO: 16)

MKVGYTEIDGKHFYFAENGEMQIGVFNTEDGFKYFAHHNEDLGNEEGEEI
SYSGILNFNNKIYYFDDSFTAVVGWKDLEDGSKYYFDEDTAEAYIGLSLI
NDGQYYFNDDGIMQVGFVTINDKVFYFSDSGIIESGVQNIDDNYFYIDDN
GIVQIGVFDTSDGYKYFAPANTVNDNIYGQAVEYSGLVRVGEDVYYFGET
YTIETGWIYDMENESDKYYFNPETKKACKGINLIDDIKYYFDEKGIMRTG
LISFENNNYYFNENGEMQFGYINIEDKMFYFGEDGVMQIGVFNTPDGFKY
FAHQNTLDENFEGESINYTGWLDLDEKRYYFTDEYIAATGSVIIDGEEYY
FDPDTAQLVISE

SEQ ID NO: 69 Clostridium difficile f1 fragment of TcdB RBD residues 2221-2366 (of full length TcdB, SEQ ID NO: 16) with single underlined flexible linkers

GGGGGKYYFNPETKKACKGINLIDDIKYYFDEKGIMRTGLISFENNNYYF
NENGEMQFGYINIEDKMFYFGEDGVMQIGVFNTPDGFKYFAHQNTLDENF
EGESINYTGWLDLDEKRYYFTDEYIAATGSVIIDGEEYYFDPDTAQLVIS
EGGGG

SEQ ID NO: 75, SFT2.cdtA (Flagellin is fused with a cdtA fragment (SEQ ID NO: 101). The cdtA signal peptide removed and S388Y, E427A and E430A are introduced to abolish enzymatic activity compared to SEQ ID NO: 73; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLAVCNTTYKAPIERPEDFLKDKEKAKEWERKEAERIEQKLERSEKE
ALESYKKDSVEISKYSQTRNYFYDYQIEANSREKEYKELRNAISKNKIDK
PMYVYYFESPEKFAFNKVIRTENQNEISLEKFNEFKETIQNKLFKQDGFK
DISLYEPGKGDEKPTPLLMHLKLPRNTGMLPYTNTNNVSTLIEQGYSIKI
DKIVRIVIDGKHYIKAEASVVSSLDFKDDVSKGDSWGKANYNDWSNKLTP
NELADVNDYMRGGYTAINNYLISNGPVVNPNPELDSKITNIENALKREPI
PTNLTVYRRSGPQEFGLTLTSPEYDFNKLENIDAFKSKWEGQALSYPNFI
YTSIGSVNMSAFAKRKIVLRITIPKGSPGAYLSAIPGYAGAYAVLLNHGS
KFKINKIDSYKDGTITKLIVDATLIP

SEQ ID NO: 76, SFT2.ED (Flagellin is fused with cdtA enzymatic domain (SEQ ID ON: 102). The mutations of S388Y, E427A and E430A are introduced to abolish enzymatic activity compared to SEQ ID NO: 73; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLAERKEAERIEQKLERSEKEALESYKKDSVEISKYSQTRNYFYDYQ
IEANSREKEYKELRNAISKNKIDKPMYVYYFESPEKFAFNKVIRTENQNE
ISLEKFNEFKETIQNKLFKQDGFKDISLYEPGKGDEKPTPLLMHLKLPRN
TGMLPYTNTNNVSTLIEQGYSIKIDKIVRIVIDGKHYIKAEASVVSSLDF
KDDVSKGDSWGKANYNDWSNKLTPNELADVNDYMRGGYTAINNYLISNGP
VVNPNPELDSKITNIENALKREPIPTNLTVYRRSGPQEFGLTLTSPEYDF
NKLENIDAFKSKWEGQALSYPNFIYTSIGSVNMSAFAKRKIVLRITIPKG
SPGAYLSAIPGYAGAYAVLLNHGSKFKINKIDSYKDGTITKLIVDATLIP

SEQ ID NO: 77, SFT2.cdtB (Flagellin is fused with cdtB without signal peptide (SEQ ID NO: 103). The K209 is mutated to A209 (of full length cdtB, SEQ ID NO: 74) to change the protease site so that cdtB remains as precursor protein, deficient of pore forming activity; C. diff. antigen is double underlined)

MAQVINTNSLSLLTQNNLNKSQSALGTAIERLSSGLRINSAKDDAAGQAI
ANRFTANIKGLTQASRNANDGISIAQTTEGALNEINNNLQRVRELAVQSA
NSTNSQSDLDSIQAEITQRLNEIDRVSGQTQFNGVKVLAQDNTLTIQVGA
NDGETIDIDLKQINSQTLGLDSLNVQKAYDVKDTAVTTKAYANNGTTLDV
SGLDDAAIKAATGGTNGTASVTGGAVKFDADNNKYFVTIGGFTGADAAKN
GDYEVNVATDGTVTLAAGATKTTMPAGATTKTEVQELKDTPAVVSADAKN
ALIAGGVDATDANGAELVKMSYTDKNGKTIEGGYALKAGDKYYAADYDEA
TGAIKAKTTSYTAADGTTKTAANQLGGVDGKTEVVTIDGKTYNASKAAGH
DFKAQPELAEAAAKTTENPLQKIDAALAQVDALRSDLGAVQNRFNSAITN
LGNTVNNLSEARSRIEDSDYATEVSNMSRAQILQQAGTSVLAQANQVPQN
VLSLLAQTSTSNHSNKKKEIVNEDILPNNGLMGYYFTDEHFKDLKLMAPI
KDGNLKFEEKKVDKLLDKDKSDVKSIRWTGRIIPSKDGEYTLSTDRDDVL
MQVNTESTISNTLKVNMKKGKEYKVRIELQDKNLGSIDNLSSPNLYWELD
GMKKIIPEENLFLRDYSNIEKDDPFIPNNNFFDPALMSDWEDEDLDTDND
NIPDSYERNGYTIKDLIAVKWEDSFAEQGYKKYVSNYLESNTAGDPYTDY
EKASGSFDKAIKTEARDPLVAAYPIVGVGMEKLIISTNEHASTDQGKTVS
RATTNSKTESNTAGVSVNVGYQNGFTANVTTNYSHTTDNSTAVQDSNGES
WNTGLSINKGESAYINANVRYYNTGTAPMYKVTPTTNLVLDGDTLSTIKA
QENQIGNNLSPGDTYPKKGLSPLALNTMDQFSSRLIPINYDQLKKLDAGK
QIKLETTQVSGNFGTKNSSGQIVTEGNSWSDYISQIDSISASIILDTENE
SYERRVTAKNLQDPEDKTPELTIGEAIEKAFGATKKDGLLYFNDIPIDES
CVELIFDDNTANKIKDSLKTLSDKKIYNVKLERGMNILIKTPTYFTNFDD
YNNYPSTWSNVNTTNQDGLQGSANKLNGETKIKIPMSELKPYKRYVFSGY
SKDPLTSNSIIVKIKAKEEKTDYLVPEQGYTKFSYEFETTEKDSSNIEIT
LIGSGTTYLDNLSITELSSTPEILDEPEVKIPTDQEIMDAHKIYFADLNF
NPSTGNTYINGMYFAPTQTNKEALDYIQKYRVEATLQYSGFKDIGTKDKE
MRNYLGDPNQPKTNYVNLRSYFTGGENIMTYKKLRIYAITPDDRELLVLS
VD

### EXEMPLIFICATION

Toxins A and B of C. difficile can cause cell disruption and death and lead to diarrhea, colitis and sepsis. Toxins A and B have can be structural defined by domains that may be susceptible to neutralization by antibodies (FIGS. 1A and 1B). These domains are the Receptor Binding Domain (RBD), Catalytic Domain (CD), which includes the glucosyl-transferase (GT) domain, the cysteine-protease (CP) domain, and the transmembrane (TL) domain. Fusion proteins that include flagellin and the RBD domains of toxin A and B were generated. Toxins A and B were from the index strain ATCC 43255. Fusion proteins were generated in two different formats: the “R3′.2x” format, in which one antigenic domain replaces the D3 domain of flagellin (between residues 193 and 194 of SEQ ID NO: 96) and a second is fused to the C-terminus; and the C-terminal fusion format in which an antigenic domain is fused to the C-terminus of full length flagellin (SEQ ID NO: 54). Fusion proteins were evaluated in in vitro assays and a mouse immunogenicity model with toxin neutralization being one of the primary readouts.

Example 1: R3′.2x and C-terminal flagellin fusion proteins utilizing Receptor Binding Domains (RBD) of Toxin A or B are antigenic, retain TLR5 activity and generate Toxin neutralizing antibodies in a mouse model.

### Methods

### Cloning of Fusion Proteins

Fusion proteins were generated employing standard molecular cloning techniques well known and established. DNA encoding elements of the Receptor Binding Domains (RBD) based on Toxins A (SEQ ID NO: 60) and B (SEQ ID NO: 67) were synthesized by an outside vendor (Genewiz Inc.). Fusion protein HL821 (SEQ ID NO: 9) was generated by inserting a small fragment of the C-terminal substrate binding domain of TcdA (f1 fragment, SEQ ID NO: 62) between residues 193 and 194 of R3′ flagellin (SEQ ID NO: 96), and a second antigen containing 14 repeat elements of RBD-A1 (SEQ ID NO: 61) fused to the C-terminus of R3′ flagellin (SEQ ID NO: 96). Fusion protein HL822 (SEQ ID NO: 10) was generated by inserting a small fragment of the C-terminal substrate binding domain of TcdB (f1 fragment, SEQ ID NO: 69) between residues 193 and 194 of R3′ flagellin (SEQ ID NO: 96), and a second antigen containing 16 repeat elements of RBD-B1 (SEQ ID NO: 68) was fused to the C-terminus of R3′ flagellin (SEQ ID NO: 96). Another antigen included the entire (also referred to herein as “complete”) RBD fused to the C-terminus of flagellin. For example, complete RBD-A2 (SEQ ID NO: 60) corresponding to amino acid residues 1812-2710 of TcdA (SEQ ID NO: 15) was used to generate the fusion protein referred to as HL928 (SEQ ID NO: 25), and complete RBD-B2 (SEQ ID NO: 67) corresponding to amino acid residues 1814-2366 of TcdB (SEQ ID NO: 16) was used to generate the fusion protein referred to as HL929 (SEQ ID NO: 26).

The flagellin sequence of S. typhimurium (SEQ ID NO: 96 for R3′.2x format or SEQ ID NO: 54 for C-terminal fusion format), and C. difficile RBD domains were amplified separately by primary PCR, and fused together by secondary PCR. The final PCR product was digested with NdeI and XhoI restriction enzymes and ligated to the pET24a plasmid. The recombinant DNA sequences were confirmed by sequencing. Plasmid with DNA constructs were transformed into E. coli BLR DE3 strain for expression.

### Protein Purification

Cells were cultured and protein production was induced using IPTG using well established techniques. After further culture, cell paste was prepared and lysed. Protein was refolded by rapid dilution and purified via standard methods of column chromatography.

### Fusion Protein Testing Strategy

In order to assess the antigenicity and immunogenicity of flagellin fusion protein that include C. difficile antigens, both in vitro and in vivo tests were performed. The in vitro tests were of three types: Capture ELISA, in vitro TLR5 and TLR5-Fc competition. The in vivo study was performed in mice and lead to two readouts: Toxin A and B specific serum IgG by ELISA and Toxin A serum neutralization using the CT26 cell line.

Three formats of flagellin fusion were produced. The first fusion protein was an R3′.2x format fusion protein in which the RBD subdomain f1 (also referred to as “f1 fragment”) was inserted into R3′ flagellin (SEQ ID NO: 96) and another RBD domain is present at the C-terminus. For example, for Toxin A, the f1 fragment (SEQ ID NO: 62) and the RBD-A1 domain (SEQ ID NO: 61) were used to generate fusion protein HL821 (SEQ ID NO: 9). For Toxin B, the f1 fragment (SEQ ID NO: 69) and the RBD-B1 domain (SEQ ID NO: 8) were used to generate fusion protein HL822 (SEQ ID NO: 10). The second format is a C-terminal fusion with the entirety of the RBD domain from either Toxin A (referred to as HL928, SEQ ID NO: 25) or Toxin B (referred to as HL929, SEQ ID NO: 26), respectively. For example, the entirety of the RBD-A2 domain (SEQ ID NO: 60 corresponding to amino acid residues 1812-2710 of TcdA (SEQ ID NO: 15)) was used to generate fusion protein as HL928 (SEQ ID NO: 25). The entirety of the RBD-B2 domain (SEQ ID NO: 60 corresponding to amino acid residues 1814-2366 of TcdB (SEQ ID NO: 16)) was used to generate fusion protein as HL929 (SEQ ID NO: 26). Fusion proteins were also generated by fusion of Toxin A RBD to the carboxy terminal amino acid of flagellin lacking domains 2 and 3 (STF2Δ) to generate SEQ ID NO: 27 and fusion of Toxin B RBD to the carboxy terminal amino acid of flagellin lacking domains 2 and 3 (STF2Δ) to generate SEQ ID NO: 28.

### Capture ELISA

For the Capture ELISA, a “sandwich” format was developed in which commercial polyclonal serum against either Toxin A or B (egg IgY from Gallus Immunotech) was coated on the plate. After washing and blocking (Superblock Tween 20, ThermoFisher), serially diluted fusion proteins were added to the plate and incubated for one hour. After another wash, fusion proteins were bound by polyclonal goat anti-flagellin serum (Covance Research Products) and detected with rabbit anti-goat IgG conjugated to HRP (Jackson ImmunoResearch). The plates were developed with TMB (ThermoFisher) and stopped with 1 M H2SO4 (J. T. Baker). Plates were read at 450 nm on a Spectramax reader and analyzed using Softmax 5.4 (Molecular Devices).

In vitro TLR5 assay

The in vitro TLR5 assay used human TLR5 HEK293 cells to measure dose-dependent IL-8 release. Cells were plated at 1×10⁴ cells per well and exposed to serially diluted fusion proteins for about 16 to about 20 hours. At that point the supernatant was tested for IL-8 using an ELISA, including a standard curve (BD Bioscience). Fusion proteins that include C. difficile antigens were compared to influenza HA fusion proteins with a similar format (e.g., R32x or C-terminal fusions). As there is a consistent discontinuity in the shape of the resulting dose response curves, determining an exact midpoint is not possible. Therefore, the overall curve appearance including upper and lower asymptotes, the slope in the dynamic part of the curve, and the approximate mid-point, are all used to compare vaccines with each other and controls.

### TLR5-Fc Competition Assay

The TR5-Fc competition ELISA used a soluble form of mouse TLR5 expressed as a fusion protein with the Fc portion of mouse IgG (R&D Systems). In the assay, serial dilutions of fusion proteins were mixed with a constant amount of TLR5-Fc and incubated for about one hour. The mix was then added to a plate coated with flagellin. TLR5-Fc not bound to the vaccine was captured on the plate and detected using rabbit anti-mouse IgG HRP, TMB and H2SO4. Different fusion proteins were compared to each other and to controls using the maximum and mid-point of the curves.

In vivo Studies: Mouse Serum ELISA and Toxin A Neutralization Test

In vivo immunogenicity studies were carried out using specific pathogen free female BALB/c mice obtained from Charles River. The in-life phase took place in a barrier colony. Mice (5 per group) were immunized with 4 μg, 10 μg or 25 μg of each fusion protein s.c. on days 0, 21, and 42 and bled on days 0, 35 and 56. As positive controls, groups of mice were injected with either Toxoid A or B (about 10 μg, List Biological Laboratories), in Montanide 720 VG adjuvant (Seppic). Chemical treatment, including formaldehyde treatment, can be used to inactivate Toxins A and B to make the respective Toxoids. Formaldehyde treatment destroys the enzymatic activity of the GTD. The resulting Toxoids can be used safely, for example, to coat ELISA plates or as control antigens for mouse immunization. A negative control group received formulation buffer (F147) only. Sera were evaluated by ELISA and in a cell-based neutralization test. For the ELISA, plates were coated with either Toxoid A or B (List Biological Laboratories). After washing and blocking plates, were exposed to serial dilutions of serum (1:5-fold steps starting at 1:50) for one hour. After washing, the remaining antibody was detected with rabbit anti-mouse IgG conjugated to HRP (Jackson ImmunoResearch). The plates were developed with TMB (ThermoFisher) and stopped with 1 M H2SO4 (J.T. Baker). Plates were read at 450 nm on a Spectramax reader and analyzed using Softmax 5.4 (Molecular Devices). OD values were converted to Toxoid-specific IgG using a standard curve of polyclonal mouse IgG run on the same plate which was fit using a 4-parameter logistic function.

For the neutralization method, CT26 mouse colon cells were treated with a cytostatic dose (100 ng/mL) of Toxin A (List Biological Laboratories) with or without dilutions of serum from vaccinated or control mice for 72 hours. Assays were performed at Eurofins Panlabs. Cell viability was detected by measuring ATP from lysed cells using a luciferase assay (Cyto-glo, Promega). The assay solution contains a cell lysis agent, which releases ATP from cells, luciferin and luciferase. Light units, read on a 96-well luminometer, are proportional to ATP released from live cells. Results are expressed either as relative light units (RLU) or as percent cell growth compared to controls: cells without toxin or serum (100% growth), and cells with toxin alone (0% growth). Chicken egg polyclonal anti-Toxin A antibody (Gallus Immunotech) was used as a positive neutralization control. This is the same antibody used in the Capture ELISA.

### Results

### Capture ELISA

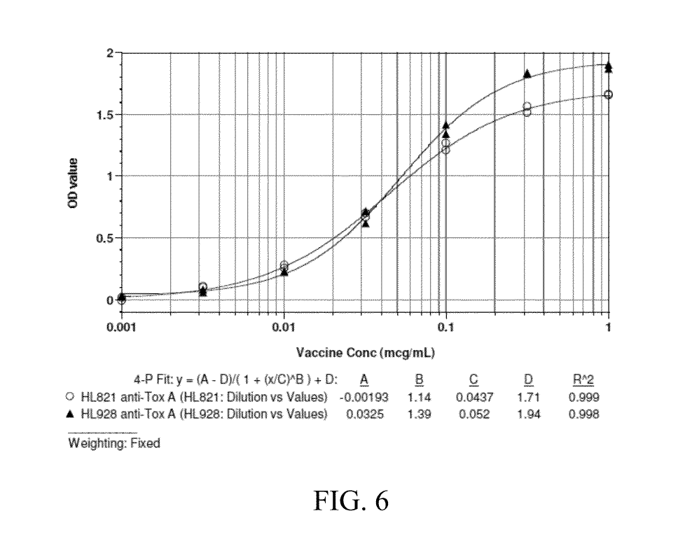

A method was established to measure the antigenicity of each fusion protein using antibodies both to the Toxin component and flagellin moiety (sandwich format). In this method, serial dilutions of fusion proteins were quantified and compared to other fusion proteins by the upper asymptote (D value of a 4-parameter logistic fit), or the curve midpoint (C value). As this method is specific for flagellin/C. difficile fusion vaccines, there is no way to compare to other controls such as Toxoids A or B or other flagellin fusion vaccines with different disease antigens, such as influenza HA. An example is shown in FIG. 6, in which the antigenicity of a fusion protein in the R3′.2x format (referred to as HL821, SEQ ID NO: 9) and a fusion protein in the C-terminal format (referred to as HL928, SEQ ID NO: 25) of Toxin A RBD domains are compared. The results indicate that the C-terminal fusion protein, which has a longer stretch of RBD sequence, has a higher upper plateau than the R3′.2x version. This is consistent with a higher degree of binding by the polyclonal anti-Toxin A serum bound to the plate. However, that the midpoint values are quite close (about 0.052 μg/mL for HL928 vs. about 0.044 μg/mL for HL821). This indicates that the two fusion proteins have a similar degree of potency: that is the presence of both Toxin A and flagellin epitopes is similar on a μg/mL basis.

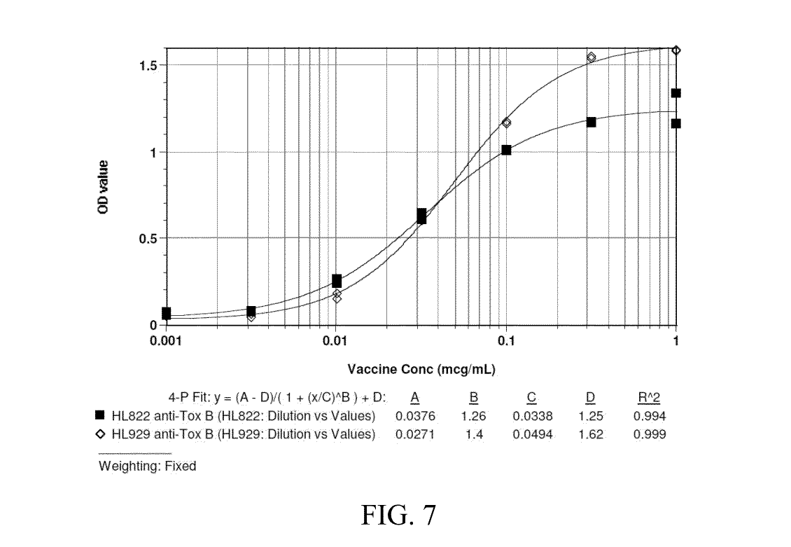

Results comparable to those of Toxin A were obtained for the Toxin B fusion proteins, as shown in FIG. 7. Fusion proteins in both R3′.2x and C-terminal formats show good dose response curves, with similar curve midpoints (about 0.044 and about 0.052 μg/mL respectively) indicating that both the Toxin B domain and the flagellin are bound by polyclonal antibodies in the same molecule. As with the fusion proteins that include Toxin A antigens, the C-terminal format fusion protein, such as the fusion protein referred to as HL929 (SEQ ID NO: 26), has a stronger maximum signal than the R3′.2x format fusion protein referred to as HL822 (SEQ ID NO: 10), consistent with the longer RBD domain being captured to a greater extent by the plate bound poly-clonal anti-Toxin antibody. As discussed above, the RBD portions used in HL822 (SEQ ID NO: 10) were the f1 fragment (SEQ ID NO: 69) and the RBD-B1 domain (SEQ ID NO: 68) while the RBD portion used in HL929 (SEQ ID NO: 26) was the entire RBD-B2 domain (SEQ ID NO: 67).

In vitro TLR5

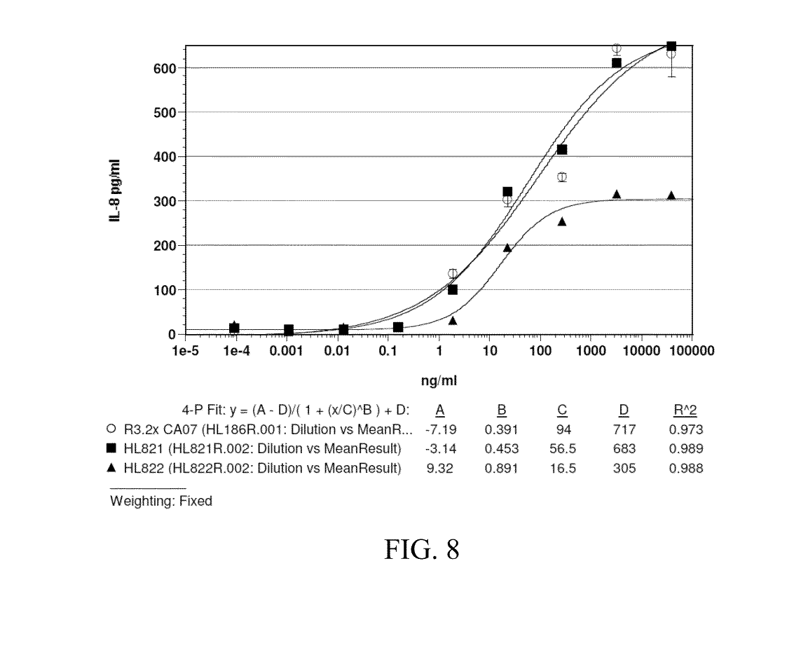

The in vitro TLR5 test is used to demonstrate that the innate stimulation function of the flagellin moiety of the fusion vaccine is intact. Fusion proteins of the invention activate TLR5. The assay has been used extensively to assess fusion proteins that include flagellin, as described, for example in U.S. application Ser. Nos. 11/714,684 and 11/714,873. Fusion proteins that include C. difficile antigens were evaluated for in vitro TLR5 activity. The R3.2x format fusion proteins for influenza A/California/07/2009 (R3.2x CA07, referred to as HL186, SEQ ID NO: 78) was used as a control for the R3′.2x format C. difficile fusion proteins referred to as HL821 (SEQ ID NO: 9, Toxin A) and HL822 (SEQ ID NO: 10, Toxin B). The results, shown in FIG. 8, indicate that the Toxin A fusion protein referred to as HL821 (SEQ ID NO: 9) behaves very similarly to fusion proteins that include flagellin and influenza proteins (referred to as R3.2x CA07, SEQ ID NO: 78), in terms of the IL-8 dose response curve. The R3.2x CA07 fusion protein (SEQ ID NO: 78) has been shown to be active in clinical trials, inducing strong neutralizing titers to the matched influenza strain. The Toxin A fusion protein curve includes the presence of a discontinuity between about 10 and about 100 ng/mL, which is characteristic of active flagellin molecules in the assay. The lower half of the curve for the Toxin B fusion protein (referred to as HL822, SEQ ID NO: 10) is similar to that of the Toxin A and the influenza fusion proteins, but the maximum IL-8 is about half of the other two.

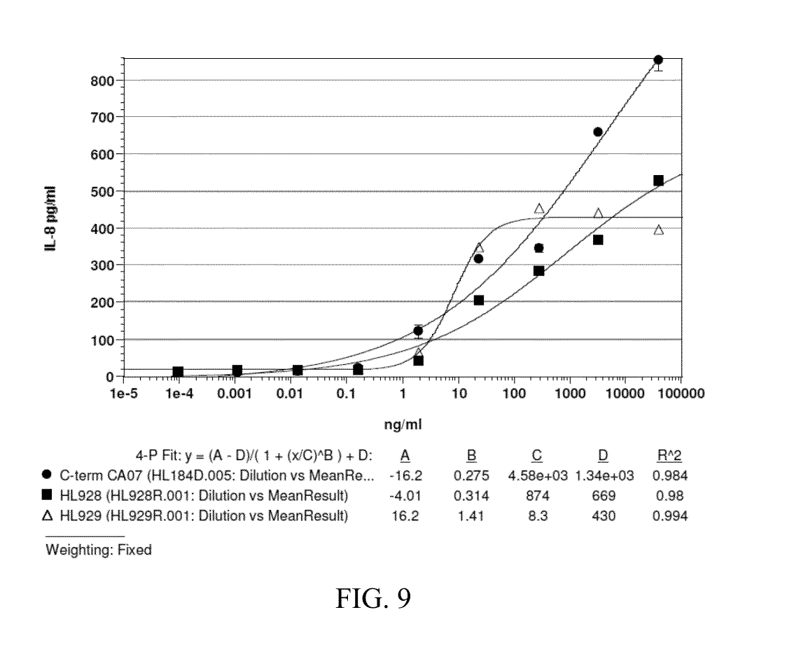

The C-terminal formats of fusion proteins that C. difficile RBD antigens for Toxins A and B were also evaluated in the in vitro TLR5 assay, in this case compared to the C-terminal influenza fusion protein that includes an antigen of A/California/07/2009 (referred to as C-term CA07, HL184, SEQ ID NO: 79). The results, shown in FIG. 9, indicate that both Toxin A and B fusion proteins are active, but are less potent than the fusion proteins of flagellin that include influenza antigens. The fusion protein that include a Toxin A antigen (referred to as HL928, SEQ ID NO: 25) follows a similar shaped curve, but with a lower slope and peak IL-8 values than the fusion protein with an influenza antigen. The fusion protein that includes a Toxin B antigen (referred to as HL929, SEQ ID NO: 26) has a similar slope to the fusion protein that includes an influenza antigen at the lower part of the curve, but like the fusion protein in the R3′.2x format that includes a Toxin B antigen, plateaus at a much lower peak IL-8 level than either the influenza fusion protein or the Toxin A fusion protein (referred to as HL928, SEQ ID NO: 25).

### TLR5-Fc Competition Assay

Further evaluation of the TLR5 binding properties of the flagellin moiety of the fusion proteins was performed using a competition ELISA format. In this assay, serial dilutions of fusion proteins were mixed with a soluble form of TLR5 genetically fused with the Fc portion of mouse IgG (TLR5-Fc, R&D Systems). After incubation, this mixture was then added to a plate precoated with flagellin. Soluble TLR5-Fc still bound to the fusion proteins was washed away in the next step while free TLR5-Fc bound the flagellin on the plate and was subsequently detected with rabbit anti-mouse IgG conjugated to HRP, TMB and H2SO4. In this assay, the TLR5-binding potency of each fusion protein was determined by the maximum TLR5-Fc signal and the midpoint of the curve (C value of a 4 parameter curve fit). As the assay is in a competition format, the lower the mid-point, the stronger the binding of a given fusion protein has to TLR5.

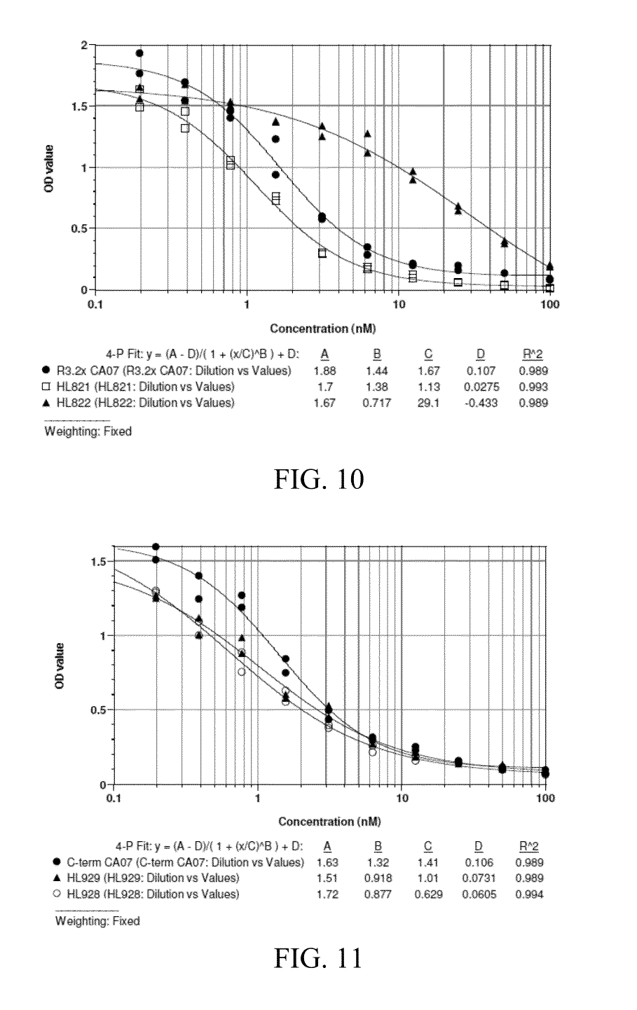

In the first experiment, the R3′.2x formats of C. difficile fusion proteins were again compared to fusion proteins that include influenza R3.2x HA CA07. The results, shown in FIG. 10, demonstrate that the Toxin A fusion protein (referred to as HL821, SEQ ID NO: 9) has a curve parallel to that of the R3.2x CA07 (SEQ ID NO: 78) fusion protein, although it is somewhat more potent. Consistent with the in vitro TLR5 assay, however, while the Toxin B fusion protein (referred to as HL822, SEQ ID NO: 10) still binds TLR5-Fc, it has a much shallower curve and a lower potency than either the Toxin A fusion protein or the HA CA07 fusion protein control.

In the second experiment, the C-terminal C. difficile fusion proteins were compared to the same format of fusion proteins that include an influenza HA CA07 antigen (SEQ ID NO: 79). The results, shown in FIG. 11, demonstrate that fusion proteins that include Toxin A (referred to as HL928, SEQ ID NO: 25) and Toxin B (referred to as HL929, SEQ ID NO: 26) have similarly shaped curves as the fusion protein with an influenza antigen, but with higher potency. These results are different than that seen with the in vitro TLR5 assay in which the IL-8 release from the C-terminal C. difficile fusion proteins is lower than that of the influenza control.

### Mouse Serum ELISA

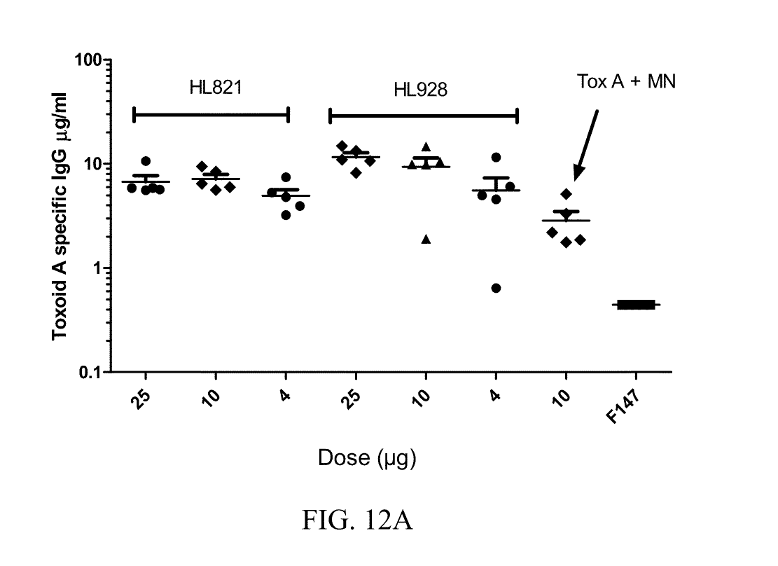

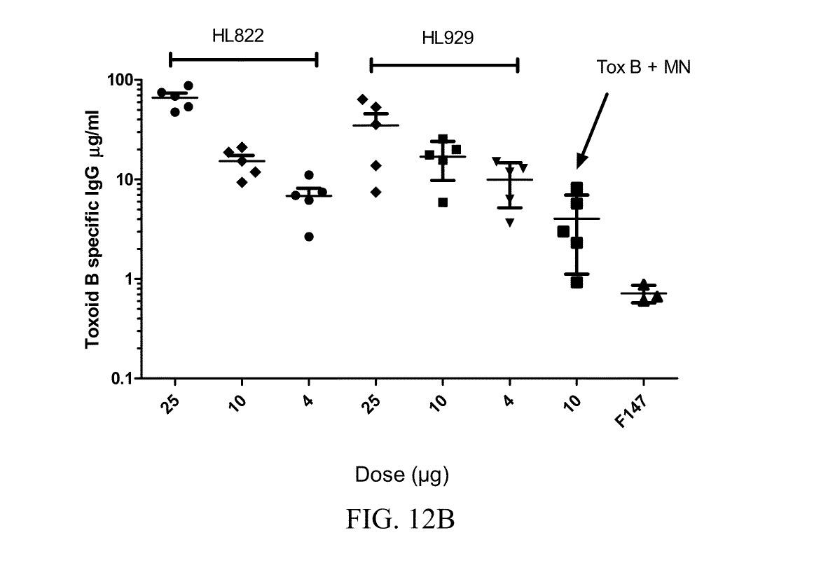

The immunogenicity of fusion proteins that include C. difficile antigens was evaluated in the BALB/c mouse model. Different doses (4 μg, 10 μg and 25 μg) of R3′.2x and C-terminal formats of Toxins A and B were used to immunize mice s.c. on days 0, 21 and 42 (prime and 2 boosts). Groups injected with fusion proteins that include Toxoids A or B (about 10 lig each) in Montanide 720 VG adjuvant were included as positive controls. A group which received formulation buffer (F 147) was included as a negative control. In order to assess antibody levels to the matched Toxoids, mice were bled on days 20 (post-prime), 35 (post-boost 1) and 56 (post-boost 2) and sera were evaluated in an ELISA. In this assay, sera were serially diluted 1:5 fold, starting at 1:50 and exposed to Toxoid-coated plates. Bound antibody was detected with rabbit anti-mouse IgG conjugated to HRP, TMB and H₂SO₄. OD values of bound antibody were converted to Toxin-specific μg/mL by interpolation against a standard curve of polyclonal mouse IgG run on each plate. Post-prime IgG results are shown in FIGS. 12A and 12B. Mice that received buffer only (F147) had only background levels of antibody (about <1 μg/mL) while nearly all mice immunized with either the R3′.2x (referred to as HL821, SEQ ID NO: 9) or C-terminal (referred to as HL928, SEQ ID NO: 25) formats of Toxin A fusion proteins (FIG. 12A) had levels about ≧1 μg/mL with some mice greater than about 10 μg/mL. There was no significant difference between R3′.2x and C-terminal formats compared at the same dose level. For both formats lower levels of antibody were observed between 4 and 10 μg groups, but no difference was observed between 10 and 25 μg groups indicating a dose plateau. The fusion proteins had higher IgG levels than the Toxoid A in adjuvant control.

In the case of fusion proteins that include toxin B antigens (FIG. 12B), higher peak IgG levels were observed than fusion proteins with toxin A (referred to as HL822, about 25 μg group mean about 66.5 μg/mL). Also, a clear dose response was seen over the entire range for both R3′.2x (referred to as HL822, SEQ ID NO: 10) and C-terminal (referred to as HL929, SEQ ID NO: 26) fusion protein formats. As with fusion proteins that include toxin A antigens, there was no significant difference between the formats at any dose level. As with fusion proteins that include toxin A antigens, both formats elicited higher antibody levels than the toxin B in adjuvant control, although these differences were not significant.

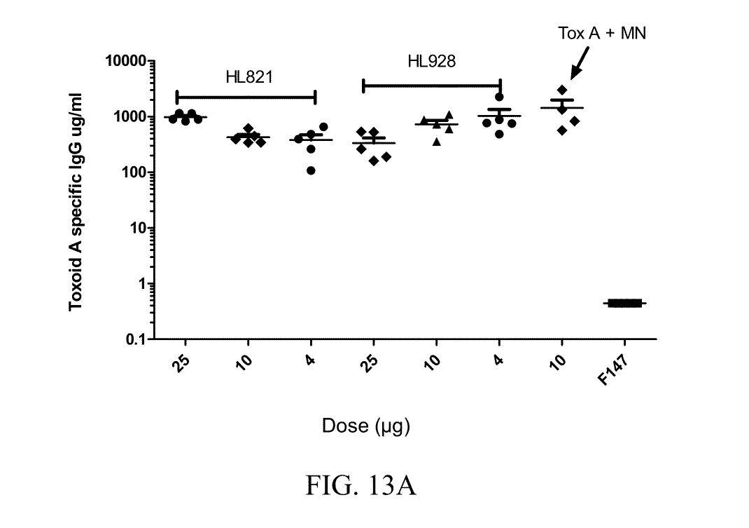

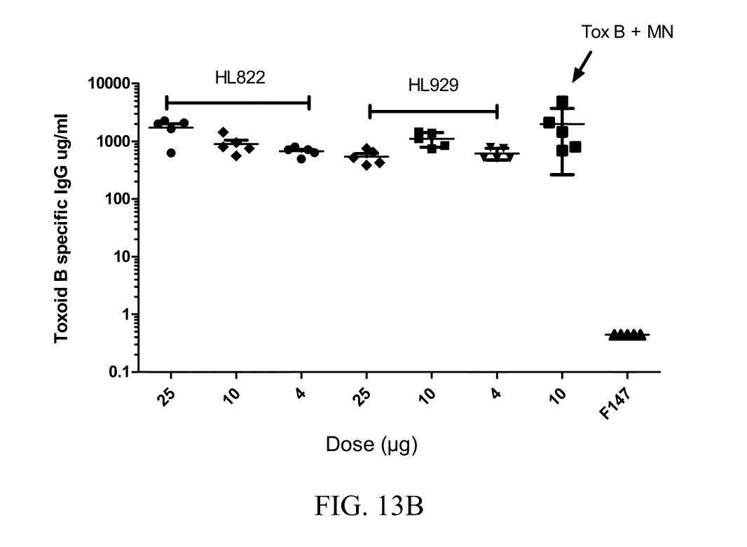

The results of the post-boost 2 serum are shown in FIGS. 13A and 13B. Anti-Toxoid A specific IgG rose to group means of about ≧1,000 μg/mL for certain groups of both R3′.2x (referred to as HL821, SEQ ID NO: 9) and C-terminal (referred to as HL928, SEQ ID NO: 25) fusion formats (FIG. 13A). The Toxoid A in adjuvant group also rose to a group mean of about >1,000 μg/ml. Anti-Toxoid B titers also rose to group means about >1,000 μg/mL for both R3′.2x (referred to as HL822, SEQ ID NO: 10) and C-terminal (referred to as HL929, SEQ ID NO: 26) formats of flagellin fusion proteins, as well as the Toxoid B plus adjuvant control (FIG. 13B).

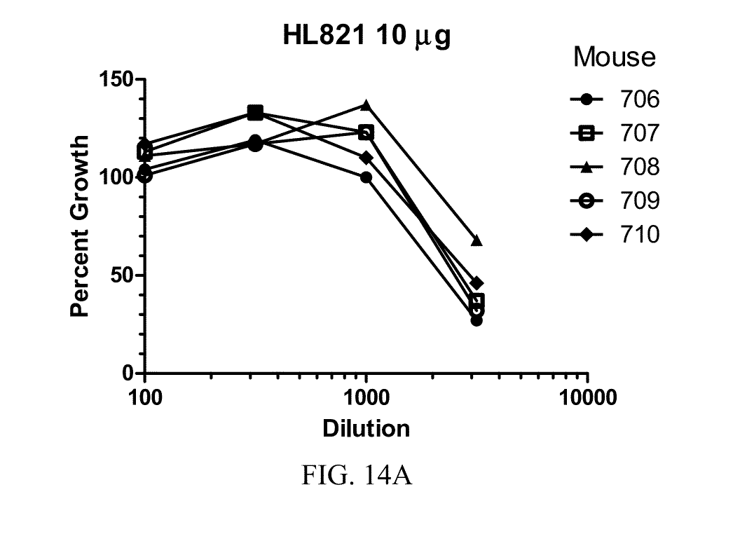

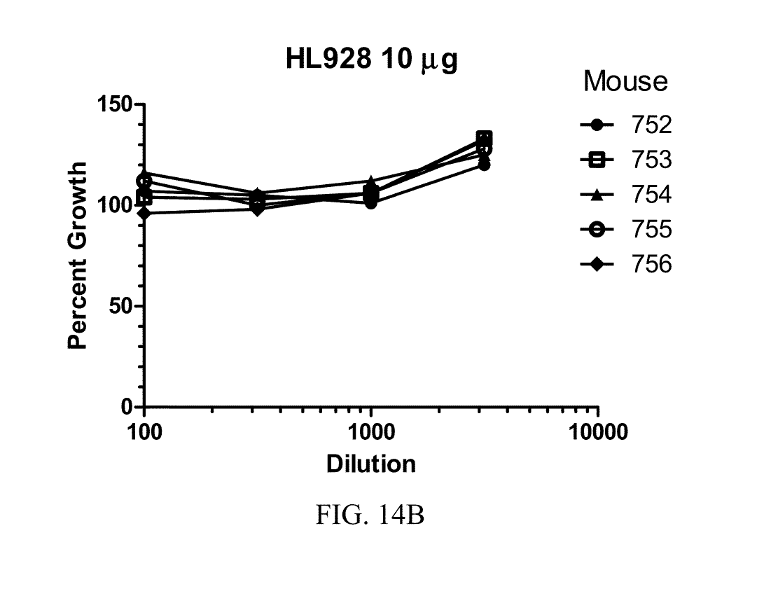

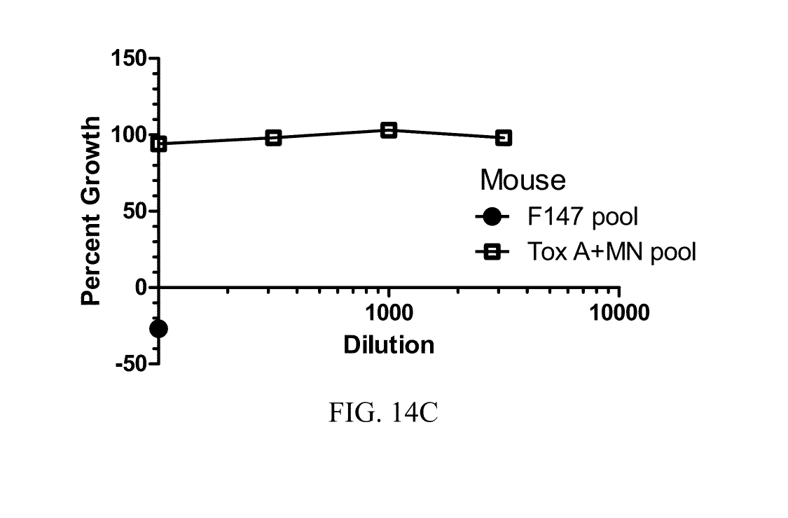

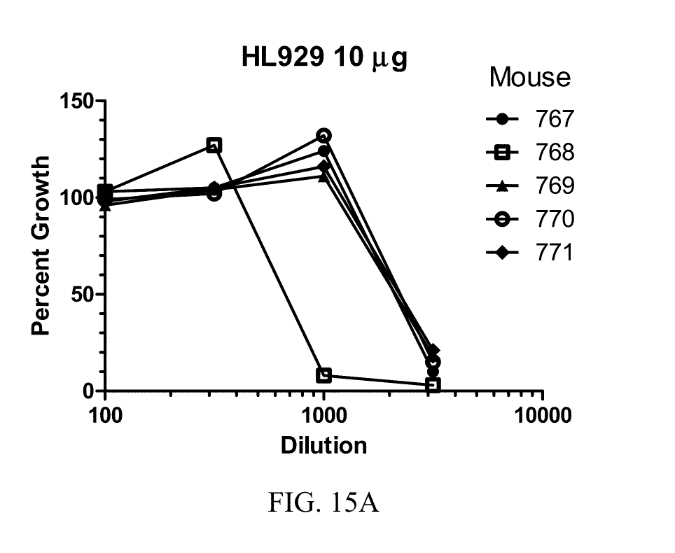

Finally, the post-boost 2 serum from mice treated with fusion proteins that include either Toxin A or B were tested for neutralizing titers using a cell-based assay. In this method, the mouse intestinal cell line CT26 is exposed to a cytostatic concentration of Toxin A or Toxin B. After about 72 hours, live cells are quantified by measuring ATP using a luciferase assay. In the assay, a single solution includes a lysis reagent, which release the ATP from the cells, luciferin substrate and luciferase enzyme. As the luciferase reaction requires ATP, light emission, measured in a 96-well plate luminometer, is proportional to ATP, which is proportional to the number of live cells. In the presence of Toxin, cells stop dividing and the level of ATP is low. In the absence of Toxin, cells continue to divide and light units, are about >10-fold higher than that seen from cells treated with Toxin. Post-boost 2 serum (diluted from 1:100 to 1:3,162) from mice immunized with 10 μg of either the R3′.2x (referred to as HL821, SEQ ID NO: 9) or C-terminal (referred to as HL928, SEQ ID NO: 25) fusion protein formats were able to neutralize the Toxin A to the level of the Vehicle control (no Toxin), when preincubated with Toxin A prior to addition to cells (FIGS. 14 A and B). The serum from mice injected with the R3′.2x format of Toxin A (HL821 SEQ ID 9) showed strong neutralizing titers with all 5 mice having plateau values (near 100%) to 1:1,000, declining at 1:3,162 (FIG. 14A). The serum from mice injected with the C terminal format of Toxin A (HL928 SEQ ID 25) also showed strong neutralizing titers with all 5 mice having plateau values (near 100%) to at least 1:3,162 (FIG. 14B). The Toxoid A plus adjuvant positive control serum pool was also able to neutralize Toxin A out to at least 1:3,162, while a pool of serum from mice injected with negative control buffer (F147, diluted 1:100) was equivalent to the Cell control (about 0% growth) (FIG. 14C). Further dilution of the HL928 post-boost 2 serum, however, will determine true values of 50% titers.

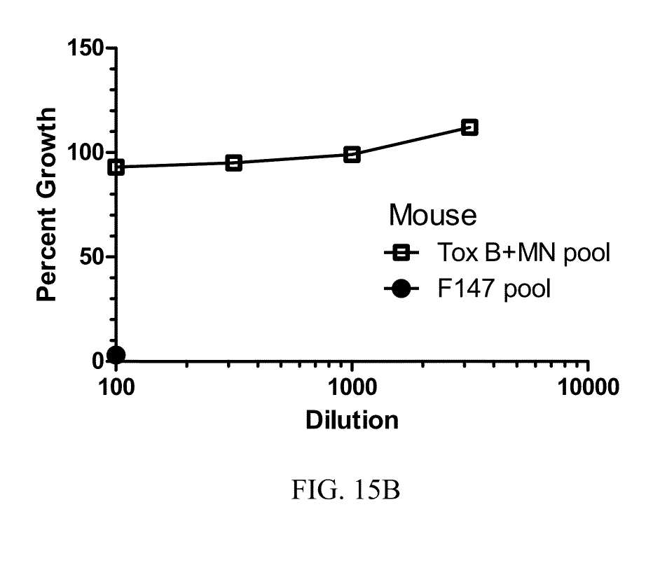

Post-boost 2 serum (diluted from 1:100 to 1:3,162) from mice immunized with 10 μg of the Toxin B C-terminal (referred to as HL929, SEQ ID NO: 26) fusion protein format was able to neutralize the Toxin B to the level of the Vehicle control (no Toxin), when preincubated with Toxin B prior to addition to cells (FIG. 15A). The serum from mice injected with the C terminal format of Toxin B (HL929, SEQ ID NO: 26) showed strong neutralizing titers with 4 of 5 mice having plateau values (near 100%) to 1:1,000, with one mouse declining to 0% at 1:1,000 and the other 4 declining at 1:3,162 (FIG. 15A). The Toxoid B plus adjuvant positive control serum pool was also able to neutralize Toxin B out to at least 1:3,162, while a pool of serum from mice injected with buffer (F 147 as a negative control, diluted 1:100) was equivalent to the Cell control (about 0% growth) (FIG. 15B).

### Conclusion

Genetic fusion of elements of the RBD of either Toxin A or Toxin B of C. difficile with flagellin, in either R3′.2x or C-terminal formats, creates recombinant fusion proteins with the following properties. The fusion proteins are recognized by antibodies to both C. difficile Toxins and flagellin in a Capture ELISA. The fusion proteins activate TLR5 HEK293 cells in a dose-dependent manner, causing the secretion of the cytokine IL-8, demonstrating the function of the flagellin portion of the fusion proteins. The fusion proteins compete with wild type flagellin to bind a recombinant, soluble form of mouse TLR5, in the TLR5-Fc Competition Assay. The fusion proteins are immunogenic in mice resulting both in Toxoid-specific IgG, in the Mouse Serum ELISA, and in Toxin A and B neutralizing antibodies, in the Toxin Neutralization Test. The anti-Toxin A titers observed for both R3′.2x (HL821, SEQ ID NO: 9) and C-terminal (HL928, SEQ ID NO: 25) post-boost 2 serum were ≧1:1,000 which is consistent with the range of effective titers (667 to 6,667) calculated by comparing clinically effective doses and neutralizing titers of monoclonal antibodies (Lowy, I., et al., N. Eng. J. Med. 362: 197-205 (2010); Babcock, G. J., et al., Infect. Immun. 74: 6339-47 (2006)). Toxin B titers are also consistent with the range of effective titers (222 to 2,222) calculated using Lowy, I., et al., N. Eng. J. Med. 362: 197-205 (2010) and Babcock, G. J., et al., Infect. Immun. 74: 6339-47 (2006). Fusion proteins of the invention may be useful to ameliorate Clostridium difficile associated disease, including Clostridium difficile-associated diarrhea and pseudomembranous colitis in susceptible patients.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

1. A composition comprising:
a) a first fusion protein that activates Toll-like Receptor 5 and includes flagellin fused to at least one Clostridium difficile toxin A antigen;
b) a second fusion protein that activates Toll-like Receptor 5 and includes flagellin fused to at least one Clostridium difficile toxin B antigen, and
c) a third fusion protein that activates Toll-like Receptor 5 and includes flagellin fused to at least one Clostridium difficile binary toxin antigen.

2.-7. (canceled)

8. A fusion protein comprising flagellin fused at the carboxy-terminal amino acid of the flagellin to at least one Clostridium difficile antigen selected from the group consisting of a portion or the entirety of a receptor binding domain of Clostridium difficile, a portion or the entirety of a translocation domain of Clostridium difficile and a portion of the entirety of an enzymatic domain of Clostridium difficile.

9.-12. (canceled)

13. A fusion protein comprising a flagellin protein in which at least a portion of or the entirety of domain 3 of the flagellin protein has been replaced with a first Clostridium difficile antigen and a second Clostridium difficile antigen is fused to the carboxy-terminal amino acid of the flagellin protein, wherein the fusion protein activates Toll-like Receptor 5.

14.-30. (canceled)

31. A method of treating a human, comprising the step of administering to the human at least one fusion protein that includes flagellin fused at the carboxy-terminal amino acid of the flagellin to at least one Clostridium difficile antigen selected from the group consisting of a portion or the entirety of a receptor binding domain of Clostridium difficile, a portion or the entirety of a translocation domain of Clostridium difficile and a portion of the entirety of a glucosyltransferase domain, whereby administration of the fusion protein ameliorates a Clostridium difficile associated disease.

32. The method of claim 31, whereby administration of the fusion protein to the human ameliorates the Clostridium difficile associated disease by generating neutralizing antibodies in the human to at least one Clostridium difficile antigen.

33. The method of claim 31, wherein the human administered the fusion protein is at least 49 years old.

34. The method of claim 31, wherein the human administered the fusion protein is at least about 50 years old to about 64 years old.

35. The method of claim 31, wherein the human administered the fusion protein is at least about 65 years old.

36. The method of claim 31, wherein the human administered the fusion protein is about 1 years old, about 2 years old, about 3 years old, about 4 years old, about 5 years old or about 6 years old.

37. The method of claim 31, wherein the human administered the fusion protein is between about 13 years old and about 18 years old.

38. The method of claim 31, wherein the human administered the fusion protein is a peripartum woman.

39. The method of claim 31, whereby the Clostridium difficile associated disease that is ameliorated following administration of the fusion protein is an antibiotic-associated diarrhea.

40. The method of claim 31, whereby the Clostridium difficile associated disease that is ameliorated following administration of the fusion protein is pseudomembranous colitis.

41. The method of claim 31, wherein the fusion protein is administered to the human in a single dose.

42. The method of claim 31, wherein the fusion protein is administered to the human in multiple doses.

43. The method of claim 31, wherein the fusion protein is administered to the human at a dose of at least one member selected from the group consisting of 0.5 μg, 1.0 μg, 1.5 μg, 5 μg, 10 μg, 20 μg, 40 μg and 80 μg of the fusion protein.

44. The method of claim 31, wherein the fusion protein of the composition administered to the human is associated with at least one member selected from the group consisting of a virosome and a virus-like particle.

45. The method of claim 31, wherein the flagellin of the fusion protein administered to the human is fused to the portion or the entirety of the receptor binding domain of Clostridium difficile toxin A.

46. The fusion protein of claim 31, wherein the flagellin of the fusion protein administered to the human is fused, in sequence, to a portion or the entirety of the translocation domain of Clostridium difficile toxin B and a portion or the entirety of the receptor binding domain of Clostridium difficile toxin B.

47. The fusion protein of claim 31, wherein the flagellin of the fusion protein administered to the human is fused to the portion or the entirety of the receptor binding domain of Clostridium difficile toxin B.

48. A method of treating a human, comprising the step of administering to the human at least one fusion protein that activates Toll-like Receptor 5 and includes flagellin in which at least a portion of or the entirety of domain 3 of the flagellin has been replaced with a first Clostridium difficile antigen and a second Clostridium difficile antigen is fused to the carboxy-terminal amino acid of the flagellin, whereby administration of the fusion protein ameliorates a Clostridium difficile associated disease.

49.-73. (canceled)
